(19) United States
(12) Patent Application Publication
(10) Pub. No.: US 2022/0265769 A1
(43) Pub. Date: Aug. 25, 2022

(54) COMPOSITIONS AND METHODS OF TREATMENT FOR BREAST CANCER INVOLVING A NOVEL CAPER?-MLL1 COMPLEX

(71) Applicant: Geisinger Health, Danville, PA (US)

(72) Inventors: Pavan Kumar PUVVULA, Danville, PA (US); Anne M. MOON, Danville, PA (US)

(73) Assignee: Geisinger Health, Danville, PA (US)

(21) Appl. No.: 17/627,373

(22) PCT Filed: Jul. 15, 2020
(86) PCT No.: PCT/US2020/042111

§ 371 Date: Jan. 14, 2022

Related U.S. Application Data
(60) Provisional application No. 62/874,239, filed on Jul. 15, 2019.
(60) Provisional application No. 62/935,910, filed on Nov. 15, 2019.

(51) Int. Cl. A61K 38/17; A61P 35/00; C07K 14/72

(52) U.S. Cl. CPC A61K 38/1796; A61P 35/00; C07K 14/72; C07K 2319/10

(57) ABSTRACT

The present invention relates to epigenetic therapies for the treatment of cancer. In particular embodiments, the present invention relates to CAPERα (Coactivator of AP1 and Estrogen Receptor) RRM3 domain-derived cell penetrating peptides that decrease breast cancer growth and survival yet are nontoxic to normal cells. In addition, the present invention relates to nucleic acid molecules encoding such therapeutic peptides, and vectors and host cells comprising such nucleic acid molecules. The invention further relates to methods of producing the therapeutic peptides of the invention, and to methods of using these therapeutic peptides in the treatment of disease.

### CROSS REFERENCE TO RELATED APPLICATIONS

This application claims the benefit of U.S. Prov. Appl. No. 62/874,239, filed Jul. 15, 2019, and U.S. Prov. Appl. No. 62/935,910, filed Nov. 15, 2019, each of which incorporated herein by reference in its entirety.

### FIELD OF THE INVENTION

The present invention generally relates to epigenetic therapies for the treatment of cancer. In addition, the present invention relates to nucleic acid molecules encoding such epigenetic therapies, and vectors and host cells comprising such nucleic acid molecules. The invention further relates to methods of producing the epigenetic therapies of the invention, and to methods of using these epigenetic therapies in the treatment of disease.

### BACKGROUND OF THE INVENTION

Breast cancer is a constellation of diseases, with different molecular etiologies and signatures that determine cell behaviors and risk of metastasis and death. Despite significant progress in defining molecular signatures of different subtypes of breast cancer, there are major gaps in understanding the mechanisms that regulate them.

Sequencing studies show that mutations in 137 cancer “driver” genes impact three central cellular processes: survival, genome stability and cell fate. Notably, mutations in driver genes that encode tumor suppressor transcription factors (p53), or oncogenic GTPases (KRAS) and kinases (EGFR) alter the epigenetic landscape central to the transformation process and nearly half of all cancer mutations occur in genes encoding chromatin modifiers. The resulting epigenetic changes promote cancer cell hallmarks including unregulated proliferation, escape from death or senescence, dedifferentiation, genome instability, immune system evasion and abnormal metabolism. To date, therapeutically targeting most oncoproteins or their downstream epigenetic effectors has been challenging or unsuccessful and if successful, would disrupt important processes in normal and cancer cells alike. In addition to the major gaps in understanding how global epigenome alterations trigger transformation, tumor initiation and metastasis, identifying epigenome regulators that can be specifically targeted is crucial to developing effective epigenetic therapies.

Histone modifying enzymes play vital roles in determining the configuration and transcriptional competency of chromatin. Transcriptionally poised or active chromatin regions in enhancers and promoters are enriched for H3K4 (histone 3, lysine 4) methylation (and/or H3K9 acetylation), while heterochromatin and repressed euchromatin have H3K9 or H3K27 methyl marks. Basal-like cancer subtypes, including triple-negative breast cancers, have the highest frequencies of HMT genetic alterations. Thus, histone methyltransferases (HMTs) are key epigenome regulators; their dysfunction causes, or contributes significantly, to the pathogenesis of many cancers. As such, it is desirable to identify ways to target key epigenome regulators, like HMTs, to disrupt processes in cancer cells.

The KMT2A-F genes encode a family of HMT proteins called Mixed Lineage Leukemia (MLL)1-5, SET1A and B. MLL1 was first revealed as proto-oncogene in a subset of aggressive leukemias in which KMT2A is mutated or translocated. Translocations yield a diverse collection of fusion proteins that activate MLL1. MLL1 regulates many processes in cancer cells including cell cycle, angiogenesis, estrogen signaling and others. In some breast cancers, MLL1 is required for the Senescence Associated Secretory Phenotype which facilitates tumor progression by inducing angiogenesis, epithelial-mesenchymal transition and promoting cancer stem cells. Gain-of-function p53 mutants activate overexpression of MLL1 to drive cancer phenotypes in some breast cancer cells.

Despite advances in understanding the oncogenic properties of MLL1, the mechanisms and factors that mediate its chromatin binding are largely obscure. In some cell types, transcription factors MAX or E2F6 are associated with MLL1. In itself, MLL1 protein has low intrinsic HMT activity such that efficient H3K4 methylation via MLL1 requires additional COMPASS members RbBP5, ASH2L and WDR5 in combination with context-specific subunits and RNAs. Such interactions are clinically relevant because protein-protein interactions are excellent therapeutic targets.

A-co-repressor consisting of TBX3 and CAPERα (Coactivator of AP1 and Estrogen Receptor, also known as RBM39) was previously identified as required to prevent premature senescence of primary human fibroblasts and regulates the activity of core senescence pathways via epigenetic marks. Increased CAPERα levels have been reported in breast and other cancers, and a shift from cytoplasmic to nuclear localization reportedly correlates with pre-malignant to malignant transition. Decreasing CAPERα in MCF7 ER+ breast cancer cells decreased expression of some cell cycle genes while in triple-negative breast cancer cells, it activated apoptosis. These studies do not reveal the basis of CAPERα function.

That is, although both CAPER and MLL1 are known to play a role in breast and other cancers, the magnitude of their contributions to breast cancer pathogenesis, the molecular mechanisms underlying these contributions, and their function in a master regulatory complex in multiple subtypes of breast cancer were all previously unknown.

The present invention provides epigenetic therapies that disrupt processes in cancer cells, but not in non-cancerous cells.

### SUMMARY OF THE INVENTION

One aspect of the present invention is directed to a method of treating cancer comprising administering a therapeutically effective amount of an RRM3-derived peptide of Coactivator of AP1 and Estrogen Receptor (CAPERα) to a subject in need thereof. In one embodiment, the cancer is breast cancer. In a specific embodiment, the breast cancer is estrogen receptor positive (ER+). In a further embodiment, the breast cancer is human epidermal growth factor receptor 2 positive (HER+). In yet a further embodiment, the breast cancer is triple-negative breast cancer (TNBC). In particular embodiments, the RRM3-derived peptide of CAPERα is nontoxic to noncancerous cells.

In other embodiments of the invention, the administration of the RRM3-derived peptide of CAPERα disrupts the CAPERα/mixed lineage leukemia 1 (MLL1) (CAP/MLL1) complex. In some embodiments, the administration of the RRM3-derived peptide of CAPERα regulates chromatin marks. In other embodiments, the RRM3-derived peptide of CAPERα prevents MLL1 occupancy in the CAP/MLL1 complex. In other embodiments, the RRM3-derived peptide of CAPERα inhibits the catalytic activity of the CAP/MLL1 complex. In other embodiments, the RRM3-derived peptide of CAPERα disrupts binding to the CAP/MLL1 complex by ASH2L and RbBP5. In other embodiments, the RRM3-derived peptide of CAPERα inhibits histone 3, lysine 4 (H3K4) trimethylation of target genes. In other embodiments, the RRM3-derived peptide of CAPERα inhibits histone 3, lysine 9 (H3K9) acetylation of target genes. In other embodiments, the RRM3-derived peptide of CAPERα increases expression of one or more of the following proteins: NFKβ, TNF, and interferon. In some embodiments, the RRM3-derived peptide of CAPERα results in decreases proliferation of cancer cells by about 30%-75%, depending on the cancer cell type. In other embodiments, the RRM3-derived peptide of CAPERα results in increases cell death by about 20%.

In some embodiments, the RRM3-derived peptide of CAPERα regulates transcription one or more of the following genes: BCL2L1, BCL6, JUNB, CCND1, FOXA1, FOXM1, ESR1, MYC, and GATA3. In other embodiments, the RRM3-derived peptide of CAPERα regulates transcription of one or more of the genes listed in Table 1. In yet further embodiments, the RRM3-derived peptide of CAPERα regulates transcription of one or more of the genes listed in Table 2. In yet further embodiments, the RRM3-derived peptide of CAPERα regulates transcription of breast cancer pioneer genes. In some embodiments, the RRM3-derived peptide of CAPERα upregulates the transcription of one or more of the following genes: CASPASE gene 2, CASPASE gene 3, CASPASE gene 4, CASPASE gene 6, CASPASE gene 8, CASPASE gene 9, KMT2B, KMT2C, KMT2D, SETD1A, vREL, and SETD1B.

In some embodiments, the RRM3-derived peptide of CAPERα inhibits CAP/MLL1 complex binding to chromatin bearing one or more of the following MYC, Rb, E2F1, STAT1/3, ARNT, and GABPB1/2. In other embodiments, the RRM3-derived peptide of CAPERα decreases association of MLL1, ASH2L, RbBP5, and/or WDR5 with genomic promoters.

Another aspect of the invention is directed to an RRM3-derived peptide of CAPERα conjugated to a protein transduction domain. In one embodiment, the RRM3-derived peptide of CAPERα does not include the 5′ β-sheet of the RRM3 domain of CAPERα. In another embodiment, the RRM3-derived peptide of CAPERα does not include the 5′ α-helix of the RRM3 domain of CAPERα. In another embodiment, the RRM3-derived peptide of CAPERα comprises the amino acid sequence of SEQ ID NO: 1. In another embodiment, the RRM3-derived peptide of CAPERα comprises the amino acid sequence of SEQ ID NO: 2. In another embodiment, the RRM3-derived peptide of CAPERα comprises the amino acid sequence of SEQ ID NO: 3. In another embodiment, the RRM3-derived peptide of CAPERα comprises the amino acid sequence of SEQ ID NO: 4. In another embodiment, the RRM3-derived peptide of CAPERα comprises the amino acid sequence of SEQ ID NO: 5. In other embodiments, the RRM3-derived peptide of CAPERα comprises the amino acid sequence of SEQ ID NO: 6. In other embodiments, the RRM3-derived peptide of CAPERα comprises the amino acid sequence of SEQ ID NO: 7. In other embodiments, the RRM3-derived peptide of CAPERα comprises the amino acid sequence of SEQ ID NO: 8. In another embodiment, the RRM3-derived peptide of CAPERα comprises the amino acid sequence of SEQ ID NO: 9. In other embodiments, the RRM3-derived peptide of CAPERα comprises the amino acid sequence of SEQ ID NO: 10. In other embodiments, the RRM3-derived peptide of CAPERα comprises the amino acid sequence of SEQ ID NO: 11. In some embodiments, the RRM3-derived peptide of CAPERα is conjugated with the Penetratin cell penetrating peptide. In other embodiments, the Penetratin cell penetrating peptide comprises the amino acid sequence of SEQ ID NO: 12. In other embodiments, the RRM3-derived peptide of CAPERα is conjugated to a poly-histidine peptide. In other embodiments, the RRM3-derived peptide of CAPERα is pegylated. In other embodiments, the RRM3-derived peptide of CAPERα is myristoylated.

Other aspects of the invention include nucleic acid molecules encoding the RRM3-derived peptides of CAPERα, and vectors and host cells comprising such nucleic acid molecules. Other aspects of the invention relate to methods of producing the RRM3-derived peptides of the invention, and to methods of using these RRM3-derived peptides to diagnose cancers that stand to benefit from treatment with the very same RRM3-derived peptides.

### BRIEF DESCRIPTION OF THE DRAWING FIGURES

FIG. 1. CAPERα knockdown reduces breast cancer cell growth in vivo and in vitro. A) Immunoblots (IBs) showing effective knockdown (kd) of CAPERα with shRNA transduction by lentivirus in T47D cells. B) Representative xenografts from control (Ctl) and CAPERα (CAP) shRNA transduced cells. C) Quantitative analysis of tumor weights and volumes. D) IBs showing effective CAPERα kd using siRNA in: T47D and MD-MBA231 breast cancer cells, the benign MCF10A breast line, and primary mammary epithelial cells (PME). E) Crystal violet assay of Ctl and CAPERα kd T47D, MDA-MB231, MCF10A and PME cell numbers. * indicates p<0.05 relative to control. F) 3T5 cell proliferation assay of cumulative population doublings in control and CAPERα kd cells from day measure every 3 days 0-15. These are representative curves from duplicate experiments; each point on the curve is a measurement of cell count from a single plating followed over the course of the experiment as described (Dai L, et al., Experimental hematology & oncology. 2(1):15 (2013)). G) Quantitation of % Ki67+ cells in Ctl and CAPERα kd cells. H) Quantitation of total cell number in Ctl and CAPERα kd cells. I) % viable cells in Ctl and CAPERα kd cells. In G-I * indicates p<0.005 relative to control.

FIG. 2. CAPERα differentially interacts with the histone methyltransferase MLL1 in T47D, MDA-MB231 breast carcinoma and human PMEs. A) Anti-CAPERα (top panel) and anti-MLL1 (bottom panel) IBs of immunoprecipitates from T47D, MDA-MB231 and PME cells using antibodies listed at top. Note co-IP of CAPER and MLL1 in breast cancer cells, but not PME (box). B) Reciprocal co-IP; confirms CAP/MLL1 complex is present in breast cancer cells and not PMEs. C) As in panel A except immunoprecipitates are from fresh frozen human breast and breast cancer cells. Note absence of CAP/MLL1 in normal breast (box) but presence in both ER+/luminal-like and triple-negative (TNBC)/basal-like human breast cancers (arrowheads). D) As above, but with commercially obtained tissue lysates of normal tissues, including those commonly adversely affected by standard chemotherapy. T47D lysate is shown as a positive control for these assays. E) IBs showing efficient siRNA mediated kd of MLL1 in T47D, MDA-MB231, MCF10A and PME cells. Actin is loading control. F) Crystal violet assay of Ctl and MLL1 kd in T47D, MDA-MB231, MCF10 and PME cells shows MLL1 kd also decreases number of breast cancer cells in culture, with no (MCF10A) or minimal (PME) effects on benign and normal breast cells, respectively. G) Quantitation of % Ki67+ cells in Ctl and MLL1 siRNA treated cells. H) IBs assaying levels of H3K4me3 in control and MLL1 siRNA transfected cells. H3 is loading control. Note decreased H3K4me3 levels in breast cancer cells (box), but not PMEs. I) MLL1 kd in T47D cells has no effect on levels of other activating or repressive histone marks.

FIG. 3. CAPERα directly and differentially interacts with MLL1 complex members via its RRM domains. A-C) Reciprocal co-IPs to assay CAPERα interactions with complex members in T47D (A), MDA-MB231 (B) and PME (C) cells. IP antibodies are listed at top of panel and IB antibodies at left. Boxes in C highlight lack of interactions in PMEs. D, E) Myc-tag pulldown assays in HEK293 cells. IBs show myc-tagged full length CAPERα co-IPs with MLL1. F) Coomassie staining showing GST and GST-CAPERα proteins (left) and anti-CAPERα IB showing full length GST-CAPERα protein used in GST-pulldown assay (right). G) IBs showing direct interaction of MLL1 core complex members with GST-CAPERα but not GST. H, I) IBs showing CAPERα C-terminal (H, lanes 4,5) RRM3 domain (I, lane 3, arrowhead) interacts with MLL1, ASH2L, RBBP5 while its N-terminal (H, lane 1) RRM1 (I, lane 1, black arrowhead) domain interacts with WDR5 in anti-myc pulldowns using HEK293 lysates after overexpressing different truncations (H) or domains (I) of CAPERα. IB antibodies listed on left side of the panel. FIG. 14 shows schematics of truncation and domain constructs. J) Coomassie staining showing purified domains of CAPERα. K) GST-pulldown assay with purified CAPERα domains and T47D nuclear lysate. IBs show interaction of MLL1, ASH2L, RbBP5 with purified RRM3 (arrowhead) and WDR5 with RRM1 (black arrowhead). L) IBs assaying MLL1-ASH2L; MLL1-RbBP5; and MLL1-WDR5 interactions in Ctl and CAP kd T47D cells. M, M′) IPs and IBs showing that although the amounts of MLL1 and ASH2L proteins are unchanged by CAPERα kd (black boxes in M, M′, respectively), the interaction between MLL1 and ASH2L is disrupted by CAPERα kd (box in M).

FIG. 4. CAPERα is required for MLL1 occupancy, trimethylation, and expression of known MLL1 target genes in T47D cells. A-C) ChIP-PCR of known MLL1 target promoters in T47D cells; HMGA2 and RIgG ChIP are negative controls. Boxes highlight CAPERα kd disruption of MLL1 occupancy and trimethylation of these promoters. D-F) RT-PCR assay of transcripts of above MLL1 targets. Note CAPERα or MLL1 kd only disrupts expression of these gene in breast cancer cells (boxes), not PMEs.

FIG. 5. The CAPERα/MLL1 complex (CAP/MLL1) occupies chromatin and regulates the transcriptional signature controlling T47D breast cancer cell phenotypes. A) Venn diagram showing overlap between CAPERα and MLL1 occupancy as determined by ChIP-seq. 79% of CAPERα sites are cobound by MLL1. B) Genomic distribution of CAPERα, MLL1 and CAP/MLL1 co-bound regions. See Methods section for parameters used. C) ChIP-Seq read density on CAPERα, MLL1 and CAPERα-MLL co-bound regions versus peak width. D) HOMER de novo motif finding pipeline identified this G C/A motif as the most significantly enriched motif in the ChIP-Seq cobound regions. D′, E, F) Pie charts showing proportions of GREAT GO Biologic Process (GO BP) term categories in CAP/MLL1 cobound genes (see Methods for categorization): D′, all genes; E, 5′ end cobound; F, whole gene cobound. The color key (D″) shows GO BP terms grouped into broader functional categories. Numbers in the pie chart wedges indicate the number of GO BP terms in that category. The actual BP terms for each chart are in FIGS. 15-28 numbered below. E′, F′) IGV traces of representative CAP/MLL1 occupancy patterns. E′ shows 5′ cobound gene as exemplified by a 7 kb window over the 5′ end of the proto-oncogene BCL6. F′ shows whole gene cobound as exemplified by window over the breast cancer pioneer gene GATA3. G) Volcano plot showing fold change in gene expression in T47D cells transfected with CAPERα siRNAs compared to control siRNA by RNA-Seq analysis. Dots represent differentially expressed genes (q<0.05); dark black, not significantly changed genes. H) Venn diagram showing the overlap between CAP/MLL1 cobound targets and upregulated and downregulated genes in CAPERα kd T47D cells. I) Pie chart as described above for significantly downregulated genes: cell cycle and metabolic genes are markedly overrepresented in this gene set. I′) GREAT analysis identified 23 cancer neighborhood genesets significantly overrepresented in downregulated genes. J) As in I, for downregulated, cobound genes; contributions mirror that for downregulated genes, validating the occupancy assignment algorithm K) As in I for upregulated genes K′) As for I′, only 2 cancer neighborhood gene groups were detected. L) As in J for upregulated, cobound genes.

FIG. 6. CAPERα and MLL1 core complex members occupy and regulate chromatin marks and transcription of novel target genes in T47D cells but not in PMEs. A) ChIP-PCR of MLL1 direct targets identified by ChIP-Seq and RNA-Seq in T47D cells shows occupancy of promoters by core complex members. B) ChIP-PCR in control and CAP kd T47D cells shows that CAPERα is required for other members of the complex to occupy these targets and for their H3K4 trimethylation. Arrowheads at bottom highlight lanes with altered binding due to CAPER kd. C) RT-PCR analysis of above-mentioned genes shows decreased expression after CAP kd (arrowheads at bottom highlights lanes with altered expression). D, E) As in B and C, but with MLL1 kd: MLL1 kd does not prevent CAPERα occupancy (black arrowhead) but disrupts H3K4 trimethylation and gene expression (arrowheads). F) ChIP-PCR shows these promoters are not occupied by CAPERα or MLL1 in PME cells. G) RT-PCR shows expression of these genes is unchanged by CAPERα kd (lane with black arrowhead) in PMEs.

FIG. 7. Differential expression and subcellular localization of CAPER and MLL complex members in breast cancer cells versus PMEs. A) RT-PCR analysis of CAPERα, MLL1, ASH2L, RbBP5, WDR5 transcript levels in cells listed; fold change is normalized to PMEs. Expression of all complex members is increased in breast cancer cells. B, C) Crystal violet cell proliferation and 3T5 cumulative population doublings assays in PMEs transduced with CAPERα over expressed lentivirus. D) RT-PCR analysis of cell cycle genes and novel CAP/MLL1 targets in PMEs transduced with empty and CAPERα-expressing lentivirus. E) IBs reveal differential subcellular locations of CAP/MLL1 core complex members in cytosolic and nuclear fractions of PMEs and breast cancer cells. LaminA/C shows clean partitioning of nuclear and cytosolic compartments. Note in PMEs, both CAPERα and WDR5 are restricted to cytoplasm, while other complex members are nuclear. In breast cancer cells, levels of all proteins are increased relative to PMEs and CAPERα and WDR5 are present in the nucleus.

FIG. 8. The CAPERα RRM3 domain functions as a dominant-negative to disrupt the CAP/MLL1 complex in breast cancer cells. A, A′) RT-PCR (A) and IBs (A′) showing expression of myc-tagged CAPERα RRM3 domain in T47D cells at the RNA and protein levels. B) IBs of control and anti-CAPERα IPs shows RRM3 disrupts interaction of CAPERα with MLL1, ASH21, RbBP5 in T47D cells (box). WDR5 remains associated, consistent with the fact that it interacts with CAPER via RRM1 (FIG. 3). C) Densitometric quantification of IBs from panel B. Gay panel represents IP′d quantification, black and white panel shows input. D, E) Crystal violet cell proliferation (D) and 3T5 cumulative population doublings assay (E) in empty vector versus RRM3 expression in T47D cells. F) RT-PCR of cell cycle gene expression shows RRM3 decreases expression of CCNA2, PCNA and CDK1, and increased expression of P16 consistent with decreased proliferation seen in E. G) ChIP-PCR of CAP/MLL1 target gene promoters. Boxes highlight lanes demonstrating RRM3 disrupting MLL1 occupancy and H3K4 trimethylation. H) RT-PCR analysis shows decreased expression (box) of target genes by RRM3. I, J, L, M) Crystal violet cell proliferation (I, L) and 3T5 cumulative population doublings assays (J, M) in MDA-MB231 breast cancer cells and PMEs, respectively. K, N) RT-PCR of cell cycle gene expression in RRM3 overexpressed MDA-MB231 and PME cells.

FIG. 9. Cell-penetrating CAPERα RRM3 disrupts breast cancer cell growth. A) Amino acid sequences of CPP-RRM1-myc, and RRM3-(441-500)-myc (SEQ ID NO: 33) and CPP-RRM3 (441-500)-myc (SEQ ID NO: 32) peptides. The sequence of Penetratin is in grey, myc tag in light grey and RRM3 in black. B) Coomassie staining showing GST-tagged, purified proteins. C, D) Representative light microscopic images of crystal violet stained cells taken after 3 days of treatment with 5 uM CPP-RRM3 (441-500)-myc. E, F) Crystal violet assay of cell numbers during treatment with 5 uM CPP-RRM3 (441-500) myc. G) Quantification of total number of cells before and after 3 days of treatment with CPP RRM3. H, I, J) Representative light microscopic images of crystal violet stained T47D cells taken after 3 days of treatment with peptides listed at top. K, L, M) Crystal violet assay of number T47D cells over 3 days of treatment with peptides listed. N) Quantification of total number of T47D cells before and after 3 days of treatment.

FIG. 10. RRM3-derived CPPs disrupts growth of multiple breast cancer cell subtypes but has no effect on normal cells. A) Schematic of RRM3 amino acids 441-510 (SEQ ID NO: 13). B) Bars represent the peptide regions used to generate CPPs based on deletion results in FIG. 31. C) Amino acid sequences of synthetic peptide: Penetratin, His tag and RRM3-derived peptide. D) Representative images of crystal violet stained breast cancer cells as listed at top, taken after 72 hours days of treatment with synthetic peptides at 5 μM. E) Quantification of total cell numbers of different cell types and peptide (1-5) treatments.

FIG. 11. CAPERα interacts with specific histone marks and differentially regulates histone post-translational modifications in breast cancer cells, and PMEs. A) Quantitation of colony formation in soft agar/anchorage independent growth in Ctl and CAPERα kd cells. B) RT-PCR analysis of pro-cell cycle transcripts; boxes show decreased expression. C) Total cell counts and % viable cells in after Ctl or CAPERα kd. D) Immunoblots of RBM39 and actin levels in T47D cells transfected with 3 specific siRNAs (lanes 1,2,3); siSMART pool (lane 5) and the combination of siRNA+RBM39 overexpression plasmid (lanes 6-9). E) Quantitation of % Ki67+ cells, total cell number, and % Annexin V+ cells in T47D cells transfected with Ctl, RBM39 siRNAs and RBM39 siRNA+RBM39 overexpression plasmid. Transfection combinations are listed at top. F) Total cell number in HEK293, HeLa, Jurkat, HUV-EC-C, UMUC3 and HCT116 cells after transfection with Ctl and RBM39 siRNAs. G) RBM39 interacts with multiple RNA binding proteins (RBPs) in both PME and T47D cells. Immunoprecipitations of RBM39 and immunoblotting for RBPs listed to assay for interaction between RBM39 and RBPs in PME and T47D. H-J) RBM39 regulates H3K4 trimethylation in breast cancer cells but not PMEs and interacts with specific histone marks in breast cancer cells versus PMEs. H) Immunoblots assaying bulk levels of H3K4me3 marks in cell types listed at the right of panels. Anti-H3 westerns are loading controls. H′) Immunoblots assaying bulk activating marks which are decreased (box) in CAP kd T47D cells, while repressive marks (H3K9me3, H3K27me3) are unaffected. Tubulin is the loading control. I) Immunoblots showing efficient immunoprecipitation of marked histone in T47D, MDA-MB231, and PMEs. J) Histone association assays show RBM39 co-immunoprecipitates with activating histone marks (H3K4me3 and H3K9ace) in breast cancer cells but not PMEs. Antibodies used in the immunoprecipitation are listed at top, cell types are at right, immunoblots are probed for RBM39.

FIG. 12. The CAP/MLL1 complex is present in multiple breast cancer subtypes. A) IP for CAPERα and IB for MLL1 (and CAPERα) show complex in all breast cancer cell lines tested; amount of MLL1 that coIPs varies. B) Table shows CAP/MLL1 complex present in all breast cancer cell lines tested and p53 status of each line. C) Complex is not present in normal and benign cell lines. D) Immunoblots of MLL1 and actin levels in T47D cells transfected with 2 different MLL1 specific siRNAs (lanes 2 and 3), MLL1siRNA mix (lane 4), and MLL CRISPR/Cas9 KO plasmid+ MLL HDR plasmid (lane 5). E) Quantitation of % Ki67+ cells (top panel) and total cell number (bottom panel) in T47D cells transfected with Ctl or MLL1 siRNAs.

FIG. 13. CAPERα interacts with coactivators in breast cancer cells and corepressors in primary fibroblasts. A & B) IPs/IBs to examine interaction of CAPERα with MLL1, MLL2, ASH2L, EZH2, HDAC1 and TBX3 in T47D compared to primary human fibroblasts (PHFs). C-H) IBs showing efficient IP of proteins from T47D and PHF lysates. Antibody and cell type are listed at top of each panel.

FIG. 14. CAPERα knockdown disrupts the interaction between ASH2L and MLL1. A-C) Schematics of myc- and GST-tagged CAPERα truncations and domains tested for direct interaction with MLL1 complex members in GST and myc pulldown assays. Numbers at left in A and B correspond to lane numbers in panels H and I of FIG. 3. D) IBs showing interaction between MLL1 and ASH2L is disrupted by CAPERα kd, but not RbBP5 or WDR5, confirming result in FIG. 3L. E) IB showing equal amounts of ASH2L in control and CAP kd samples. F) IBs to test for interaction between ERα (left panel) or HCF1 (right panel).

FIG. 15. CAPERα directly binds to DNA via its RRM2 domain. A) Representative electrophoretic mobility shift assays (EMSAs) of GST and GST-CAPERα with single and double stranded DNA oligos. B) EMSA for indicated promoter regions of novel target genes using 200 ng of GST protein and 0, 20, 40, 80, 160, 320 ng recombinant GST-CAPERα. C) EMSA with individual domains of CAPERα (listed at top of the panels) and “GC” and “AT” oligos (listed at bottom of the panel).

FIG. 16. Complete HOMER output de novo motifs, all cobound peaks.

FIG. 17. GREAT predicted promoter motifs, all cobound peaks.

FIG. 18. GREAT GO BP terms categorized by color as in FIG. 5 D″.

FIG. 19. As in FIG. 18 for panel E.

FIG. 20. As in FIG. 18 for panel F.

FIG. 21. As in FIG. 16, cobound promoters.

FIG. 22. As in FIG. 17, cobound promoters.

FIG. 23. As in FIG. 18, downregulated genes.

FIG. 24. As in FIG. 18, downregulated/cobound genes.

FIG. 25. GREAT MSig DB pathway enrichment analysis among downregulated and downregulated/cobound genes.

FIG. 26. As in FIG. 18, upregulated genes.

FIG. 27. As in FIG. 18, upregulated/cobound genes.

FIG. 28. As in FIG. 25, upregulated and upregulated/cobound genes.

FIG. 29. Overexpressed CAPERα is not in the nucleus. IBs showing PME cytoplasmic and nuclear fractions from lysates after transfection of empty (vector) or CAPERα expression (CAP OE) vectors.

FIG. 30. RRM1 and RRM2 have no effect on growth of T47D cells. A-D) Crystal violet cell proliferation (A, B) and 3T5 cumulative population doubling (C, D) assays in T47D cells overexpressing RRM1 and RRM2 domains. E, F) IB demonstrating that only the RRM3 domain of CAPERα disrupts the endogenous interaction between CAPERα and MLL1 in T47D cells. The RRM1 and RRM2 domains have no effect on CAPERα/MLL1 association.

FIG. 31. Key structural domains mediate the RRM3-MLL1 interaction. A) Modeling of RRM3 domain structure (amino acids: 441 to 510; SEQ ID NO: 13) using the SWISS MODEL tool and conserved domains by sequence alignment with vertebrate RRM-domaining proteins (https://www.ncbi.nlm.nih.gov/Structure/cdd/cddsrv.cgi?uid=240731) B) Top: anti-MLL1 IB to test the interaction between MLL1 and myc-tagged, deletion mutant RRM3 proteins in HEK293 cells. Bottom: IB/IP to quantitate myc-tagged proteins in lysates from HEK293 overexpressing wild type and microdeleted RRM3 domains. C) Bar graphs quantitating % viable cells after peptide treatments (peptides are numbered 1-5 as in FIG. 10) for 4 days. D) Anti-His immunoblots showing Dignam (nuclear enriched) lysates obtained from T47D cells treated with CPPs demonstrate the presence of the His-tagged peptides in the nucleus. Lanes are labeled with peptide names. Arrowhead highlights signal from the entry of CPP. E) Representative high-power images of anti-His immunocytochemical signal of T47D cells transduced with His-tagged CPP-E445-V476 and the left panel shows no anti-His primary antibody negative control, the right panel shows His-tagged CPP in cytoplasm and nucleus of all cells. F) Anti-RBM39 immunoprecipitated samples from Neg.CPP (SEQ ID NO: 12), CPP-L491-L508 (SEQ ID NO: 24), CPP-G441-V460 (SEQ ID NO: 20), and CPP-E445-V476 (SEQ ID NO: 15) treated T47D lysates assayed by immunoblotting with antibodies against MLL1, ASH2L, RbBP5. Note, CPP E445-V476 reduced the interaction between RBM39 and MLL1 complex members (lane 4, arrow; reduced interaction). G) Representative co-immunoprecipitations to show the interaction between RBM39 and its RBP interactors in Neg.CPP and CPP-E445-V476 treated T47D cell lysates. Note, CPP-E445-V476 did not affect the interaction of RBM39 with RBPs (compare lane 5 and 6). H) Treatment of breast cancer cell lines with CPP-E445-V476 disrupts the endogenous interaction between RBM39 and MLL1 in vivo. Immunoprecipitations and Immunoblots assaying RBM39-MLL1 interaction in neg.CPP and CPP-E445-V476 treated T47D, HCC1954, MDA-MB231 and ZR7530 cells show reduced interaction between MLL1 and RBM39 in vivo. (arrows highlight decreased interaction).

FIG. 32. IGV tracks showing RBM39 and MLL1 ChIP-seq occupancy patterns at gene loci. RBM39 and MLL1 occupy upstream regions, promoters and TSS regions of CCND1, DUSP6, CBX8, BCL6, BANF1 but not TGFB1 or MMP3. MLL1 specifically associates with the promoter of MAPK14.

FIG. 33. IGV tracks showing RNA-seq readout of RBM39, BCL6, CBX8, and DUSP6 in Ctl and RBM39 siRNA transfected T47D cells.

FIG. 34. CAPERα interacts with specific histone marks and differentially regulates histone post-translational modifications in breast cancer cells and primary fibroblasts. A) IBs to assay levels of histone marks in in Ctl and CAPERα kd primary human fibroblasts (PHFs). H3 is a loading control. Activating marks are unaffected while repressor marks are decreased (box) by CAP kd. B) IBs showing efficient histone IPs in PHFs. C) Histone association assays show CAPERα predominantly interacts with repressive marks in PHFs.

FIG. 35. CAPERα differentially interacts with COMPASS and Polycomb family members independent of DNA in T47D and MDA-MB231 cells. A) Co-immunoprecipitation assay to test interaction of RBM39 with COMPASS/MLL family members in T47D and MDA-MB231 cells. Boxes highlight lack of interaction, black boxes highlight interaction with MLL1, 2 and are cell-type specific. B) Evaluation of RBM39 interactions with Polycomb family transcriptional repressors in T47D and MDA-MB231 cells. Immunoblot antibodies are listed at left and immunoprecipitation antibodies are listed at top. All these negative interactions highlight the specific association between RBM39 and MLL1 complex members. C) Co-immunoprecipitation analysis between RBM39 and MLL1 components in the presence (+) or absence (−) of DNasel treatment. Shows interaction between RBM39 and MLL1 is DNA independent.

FIG. 36. Modifications to E445-V476 RRM3-derived peptide. A) T47D, B) MDA-MB231, C) MCSF10A, A) T47D breast cancer cells were treated with 5 μM of negative control CPP-His tag (1) (SEQ ID NO: 47), CPP-RRM3-E445-V476-His tag (2) (SEQ ID NO: 14), CPP-RRM3-E445-V476-His tag with D-amino acid substitutions of K447, V450, E453, K456, V460, H462, V465, N468, Q471, and V474 (3) (SEQ ID NO: 17), pegylated CPP-RRM3-E445-V476-His tag (4) (SEQ ID NO: 18), or myristoylated CPP-RRM3-E445-V476-His tag (5) (SEQ ID NO: 19) for 4 days. B, C) MDA-MB231 breast cancer and non-tumorigenic MCF10A breast cells were likewise treated with 5 μM of negative control CPP-His tag (1), CPP-RRM3-E445-V476-His tag (2), CPP-RRM3-E445-V476-His tag with D-amino acid substitutions of K447, V450, E453, K456, V460, H462, V465, N468, Q471, and V474 (3), pegylated CPP-RRM3-E445-V476-His tag (4), or myristoylated CPP-RRM3-E445-V476-His tag (5) for 4 days. After 4 days, total cell number was quantified.

FIG. 37. Treatment with different RRM3-derived peptides results in varying effects on total cell count. A) T47D, B) MDA-MB231, C) MCF10A, A) T47D breast cancer cells were treated with 5 μM of negative control CPP-His tag (1) (SEQ ID NO: 47), CPP-RRM3-E445-V476-His tag (2) (SEQ ID NO: 14), CPP-RRM3-G441-V460-His tag (3) (SEQ ID NO: 21), CPP-RRM3-I461-5480-His tag (4) (SEQ ID NO: 23), CPP-RRM3-L491-L508-His tag (5) (SEQ ID NO: 25), both CPP-RRM3-G441-V460-His tag and CPP-RRM3-I461-5480-His tag (6), both CPP-RRM3-I461-5480-His tag and CPP-RRM3-L491-L508-His tag (7), or both CPP-RRM3-G441-V460-His tag and CPP-RRM3-L491-L508-His tag (8), for 4 days. B, C) MDA-MB231 breast cancer and non-tumorigenic MCF10A breast cells were likewise treated with 5 μM of negative control CPP-His tag (1), CPP-RRM3-E445-V476-His tag (2), CPP-RRM3-G441-V460-His tag (3), CPP-RRM3-I461-S480-His tag (4), CPP-RRM3-L491-L508-His tag (5), both CPP-RRM3-G441-V460-His tag and CPP-RRM3-I461-S480-His tag (6), both CPP-RRM3-I461-S480-His tag and CPP-RRM3-L491-L508-His tag (7), or both CPP-RRM3-G441-V460-His tag and CPP-RRM3-L491-L508-His tag (8), for 4 days. After 4 days, total cell number was quantified.

FIG. 38. Effect on total cell count by different lipid treatment of RRM3-derived peptides. A) T47D, B) MDA-MB231, C) MCF10A, A) T47D breast cancer cells were treated with 5 μM of negative control CPP-His tag (1), CPP-RRM3-E445-V476-His tag (2), CPP-RRM3-E445-V476-His tag in liposomes prepared by the procedure recited for Reagent 89850 (Pierce) (3), or CPP-RRM3-E445-V476-His tag coated with Stearyl-R8 reagent from lifetein (4), for 4 days. B, C) MDA-MB231 breast cancer and non-tumorigenic MCF10A breast cells were likewise treated with 5 μM of negative control CPP-His tag (1), CPP-RRM3-E445-V476-His tag (2), CPP-RRM3-E445-V476-His tag in liposomes prepared by the procedure recited for Reagent 89850 (Pierce) (3), or CPP-RRM3-E445-V476-His tag coated with Stearyl-R8 reagent from lifetein (4), for 4 days. After 4 days, total cell number was quantified.

FIG. 39. Effect of CPP-RRM3-E445-V476-His tag on non-breast cells. UMUC3 human bladder cancer (columns 1 and 2), HCT116 human colon cancer (columns 3 and 4), DU145 human prostate cancer (columns 5 and 6), and HT1080 fibrosarcoma cells (columns 7 and 8) were treated with either 5 μM of negative control CPP-His tag or CPP-RRM3-E445-V476-His tag. After 4 days, total cell number was quantified.

FIG. 40. Modifications to tags of E445-V476 RRM3-derived peptide. A) T47D, B) MDA-MB231, C) MCF10A, A) T47D breast cancer cells were treated with 5 μM of negative control CPP-His tag (1) (SEQ ID NO: 47), CPP-RRM3-E445-V476-His tag (2) (SEQ ID NO: 14), KLF peptide-RRM3-E445-V476-His tag (3) (SEQ ID NO: 29), or TAT peptide-RRM3-E445-V476-His tag (4) (SEQ ID NO: 27), for 4 days. B, C) MDA-MB231 breast cancer and non-tumorigenic MCF10A breast cells were likewise treated with 5 μM of negative control CPP-His tag (1), CPP-RRM3-E445-V476-His tag (2), KLF peptide-RRM3-E445-V476-His tag (3), or TAT peptide-RRM3-E445-V476-His tag (4), for 4 days. After 4 days, total cell number was quantified.

FIG. 41. Effect of CCP-RRM3-E445-V476-His tag on luminal and triple negative breast cancer cells. A) Breast cancer luminal HCC1428 (columns 1 and 2), ZR7530 (columns 3 and 4), and T47D cells (columns 5 and 6) were treated with either 5 μM of negative control CPP-His tag or CPP-RRM3-E445-V476-His tag. B) Triple-negative breast cancer MDA-MB157 (columns 1 and 2), HCC1806 (columns 3 and 4), HCC1143 cells (columns 5 and 6) were treated with either 5 μM of negative control CPP-His tag or CPP-RRM3-E445-V476-His tag. After 4 days, total cell number was quantified.

FIG. 42. RBM39 and MLL1 core complex members require RBM39 to occupy and regulate chromatin marks and transcription of novel target genes in T47D cells but not in PMEs. A, B) RBM39, MLL1, ASH2L, RbBP5 and WDR5 ChIP-PCR for indicated genes were performed in T47D cells. C) RBM39 qChIP-PCR on indicated gene regions. Primer positions correspond to the distance from TSS on X-axis. (—) and (+) indicate distance up- or downstream of TSS respectively. RBM39 chromatin association is plotted as fold enrichment relative to isotype control. Biological triplicates were used to generate the s.d. (standard deviation) which is represented as error bars. D, J) RBM39, MLL1, ASH2L, RbBP5, WDR5 and H3K4me3 qChIP-PCR for indicated gene promoters on chromatin from T47D cells after 2 days of Ctl and RBM39 si treatment. E) RBM39, MLL1 and H3K4me3 qChIP-PCR for indicated genes as above after MLL1 si treatment in T47D cells. F) RBM39 and MLL1 qChIP-PCR of PME chromatin. G, H, I, K, L) qRT-PCR analysis of transcript levels of genes in total RNA prepared after 2 days of siRNA treatment.

FIG. 43. Conditional ablation of Caperα in mammary epithelium prevents tumor formation in the C3tag genetically engineered mouse model of breast cancer. The floxed allele of Caperα was created in the Moon laboratory (unpublished). This allele is null for Caperα after Cre-mediated recombination. The C3tag model of breast cancer was generated as described in: Maroulakou IG. et al., Proc Natl Acad Sci USA 91(23):11236-40 (1994). The strain was purchased from the Jackson Labs for this study. Stock No: 013591|C3(1)-TAg, C3(1)/Tag. The LGBCre (called BCre in the figure) transgenic mouse line was purchased from Jackson labs for this study. Stock No: 017836Tg(LGB-cre)74Acl/J. The strain expresses Cre recombinase in the mammary epithelium during lactation. These mice bearing these three alleles were intercrossed to generate control (Caper flox/flox;C3Tag) and conditional ablation (Caper flox/flox;C3Tag,BCre) female mice which were monitored for tumor formation. The mammary conditional ablation of Caperα was driven by Cre activity in the mammary epithelium during lactation. Control female B6666 (left panel) was sacrificed at 7 months due to the presence of multiple large bilateral mammary tumors and cachexia. This animal is representative of a total of 4 control females examined. Conditional ablation female B6645 (right panel) was healthy at 7 months and had undergone 3 rounds of lactation at the time of sacrifice. Only 2 small tumors were detected by dissection post-mortem. This animal is representative of 3 conditional ablation females examined.

### DETAILED DESCRIPTION OF THE INVENTION

The present inventors are the first to explain the molecular mechanisms and interacting proteins for CAPERα in multiple breast cancer subtypes and demonstrate critical differences in its function in breast cancer and normal breast epithelial cells. The present inventors discovered a novel epigenetic regulatory complex between CAPERα and MLL1 (CAP/MLL1) in breast cancer; this complex does not exist in normal breast cells. The present inventors uncovered inherent DNA binding activity in CAPERα and show that binding and H3K4 trimethylation (H3K4me3) of target chromatin by MLL1 requires CAPERα occupancy in multiple breast cancer cell types. Consistent with this, RNA- and ChIP-Seq experiments show decreased global H3K4me3 marks and dysregulation of thousands of CAP/MLL1 direct transcriptional targets after CAPERα knockdown including master regulators such as MYC, GATA3 and FOXA1. Of therapeutic relevance, disruption of the CAP/MLL1 complex with dominant-negative cell penetrating peptides derived from the RRM3 domain of CAPERα normalized proliferation and gene expression of breast cancer cells yet had no effect on normal cells. These findings reveal a novel epigenetic regulator of the transcriptional signatures of breast cancer cells, the molecular mechanisms underpinning CAPERα and MLL1 function in breast cancer, and new avenues for cancer cell-specific epigenetic therapies based on CAP/MLL1 complex disruption

As discussed above, CAP/MLL1 is a critical complex that functions near the top of the transcriptional regulatory hierarchy in breast cancer cells. It controls chromatin structure and transcriptional signatures upstream of abnormal proliferation and other cancer hallmarks in multiple breast cancer subtypes. As discussed in further detail below, a key finding by the inventors is that the complex is not present in primary mammary epithelial cells (and numerous other normal tissues) such that CAPERα knock-down (kd) or complex disruption has no effect in these cells. DNA-binding and protein-protein interaction studies conducted by the inventors reveal new and distinct functions of individual CAPERα functional domains in CAP/MLL1 complex formation, stability, and DNA-binding, and elucidate a new transcription regulation function at the level of chromatin structure distinct from CAPERα's previously known coactivator and splicing regulator functions. Dominant-negative CAPERα RRM3-derived CPPs disrupt the complex and normalize proliferation and gene expression of multiple subtypes of breast cancer cells.

As discussed in further detail below, genome-wide studies conducted by the inventors revealed that the vast repertoire of CAP/MLL1-regulated genes and pathways is critical for obtaining and/or maintaining cancer hallmarks in breast cancer cells. In addition to directly regulating many cell cycle, oncogenes, tumor suppressors and housekeeping genes, the inventors found that disruption of CAP/MLL1 profoundly alters the metabolic and inflammatory transcriptomes of breast cancer cells and activates proapoptotic cascades, all of which contribute to decreased proliferation and viability. This metabolic response and enrichment of GABPA/B and Myc motifs in the ChIP-Seq data are concordant with work by Kang and colleagues showing that CAPERα has a highly conserved role integrating energy homeostasis and mitochondrial function in hepatocytes via ERRα-GABPA and NFKβ/Myc pathways (Kang Y K, et al., PLoS Genet. 11(4):e1005116 (2015)); many of the CAPERα regulated genes reported by Kang et. al. are also direct targets of CAP/MLL1 in breast cancer cells.

As discussed in further detail below, the inventors found a novel molecular function for CAPERα as a transcription factor with DNA-binding activity critical to recruit MLL1 and other COMPASS epigenetic regulators to chromatin of target promoters. Despite previous studies showing context-dependent co-activator or -repressor function with DNA-binding cofactors (ESR1, AP1, TBX3, REL) (Kumar P P, et al., eLife. e02805 (2014); Dutta J, et al., J Virol. 82(21):10792-802 (2008); Jung D J, et al., J Biol Chem. 277(2):1229-34 (2002)) the inventors found that CAPERα does not interact with ESR1 in breast cancer cells rather, CAP/MLL1 regulates pioneer factor expression upstream of estrogen receptor signaling in ER+ T47D cells and also functions in hormone receptor negative breast cancer subtypes.

As discussed in further detail below, the inventors were the first to obtain important insight into how molecular functions of CAPERα are mediated by different domains. DNA binding is mediated by RRM2. RRM1 is necessary for interaction with WDR5, the RNA binding pocket for the MLL1 core complex. RRM3 mediates interaction with MLL1, ASH2L and RbBP5 and only RRM3 decreases breast cancer cell proliferation/survival and cell cycle gene expression by disrupting CAP/MLL1 in a dominant-negative manner.

Cellular fractionation studies provided insight into why the CAP/MLL1 complex can form in breast cancer cells (where all components are present in the nucleus) but not in PMEs (where CAPERα and WDR5 are restricted to the cytoplasm).

Several groups had reported anti-cancer strategies targeting CAPERα or MLL1 (Grembecka J, et al., Nature chemical biology. 8(3):277-84 (2012); Senisterra G, et al., Biochem J. 449(1):151-9 (2013); Wang E, et al., Cancer Cell. 35(3):369-84 e7 (2019); Thomas R, et al., Cancer Cell. 35(3):337-9 (2019)). However, both proteins are ubiquitously expressed and have important roles in most normal cells. In fact, the inventors determined that Caperα is required for survival of numerous progenitor populations in the developing embryo and complete ablation of Caperα in mice causes early embryonic lethality. Because the CAP/MLL1 complex is not present in the normal cells tested, it presents a unique, cancer cell-specific therapeutic target such that RRM3-derived cell penetrating peptides disrupt the complex and inhibit proliferation and survival of multiple subtypes of breast cancer cells but have no effect on normal mammary epithelial cells. The inventors have also undertaken the next step of preclinical testing of these peptides using mouse models of breast cancer and patient-derived tumor xenografts to assess their interference with tumor formation, progression and metastasis in vivo. These results serve for the strategy of CAP/MLL1 disruption with cell-penetrating dominant-negatives as efficacious, selective, and nontoxic for new cancer therapy.

### Definitions

Unless defined otherwise, all technical and scientific terms used herein generally have the same meaning as commonly understood by one of ordinary skill in the art. Generally, the nomenclature used herein and the laboratory procedures in cell culture, molecular genetics, nucleic acid chemistry and hybridization described below are those well-known and commonly employed in the art. Standard techniques and procedures are generally performed according to conventional methods in the art and various general references (see generally, Sambrook et al. Molecular Cloning: A Laboratory Manual, 2nd ed. (1989) Cold Spring Harbor Laboratory Press, Cold Spring Harbor, N.Y., which is incorporated herein by reference), which are provided throughout this document.

### RRM3-Derived Peptides

The RRM3-derived peptides of the invention comprise polypeptides and fragments thereof. As used herein, term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

A polypeptide of the invention may be of a size of about 3 or more, 5 or more, 10 or more, 20 or more, 25 or more, 50 or more, 75 or more, 100 or more, 200 or more amino acids. Polypeptides may have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations, and are referred to as unfolded.

By an “isolated” polypeptide or a variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated for purposed of the invention, as are native or recombinant polypeptides which have been separated, fractionated, or partially or substantially purified by any suitable technique.

Also included as polypeptides of the present invention are derivatives, analogs, or variants of the foregoing polypeptides, and any combination thereof. The terms “variant,” “derivative” and “analog” when referring to polypeptides of the present invention include any polypeptides that retain at least some of the biological, antigenic, or immunogenic properties of the corresponding native polypeptide. Variants of polypeptides of the present invention include polypeptides with altered amino acid sequences due to amino acid substitutions, deletions, or insertions. Variants may occur naturally or be non-naturally occurring. Non-naturally occurring variants may be produced using art-known mutagenesis techniques. Variant polypeptides may comprise conservative or non-conservative amino acid substitutions, deletions or additions. Derivatives of polypeptides of the present invention, are polypeptides which have been altered so as to exhibit additional features not found on the native polypeptide. Examples include fusion proteins. Variant polypeptides may also be referred to herein as “polypeptide analogs.” As used herein a “derivative” of a polypeptide refers to a subject polypeptide having one or more residues chemically derivatized by reaction of a functional side group. Also included as “derivatives” are those peptides which contain one or more naturally occurring amino acid derivatives of the twenty standard amino acids. For example, 4-hydroxyproline may be substituted for proline; 5-hydroxylysine may be substituted for lysine; 3-methylhistidine may be substituted for histidine; homoserine may be substituted for serine; and ornithine may be substituted for lysine.

Alternatively, recombinant variants encoding these same or similar polypeptides can be synthesized or selected by making use of the “redundancy” in the genetic code. Various codon substitutions, such as the silent changes which produce various restriction sites, may be introduced to optimize cloning into a plasmid or viral vector or expression in a particular prokaryotic or eukaryotic system. Mutations in the polynucleotide sequence maybe reflected in the polypeptide or domains of other peptides added to the polypeptide to modify the properties of any part of the polypeptide, to change characteristics such as ligand-binding affinities, interchain affinities, or degradation/turnover rate.

Preferably, amino acid “substitutions” are the result of replacing one amino acid with another amino acid having similar structural and/or chemical properties, i.e., conservative amino acid replacements. “Conservative” amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino, acids include arginine, lysine, and histidine; and negatively charged (acidic) amino acids include aspartic acid and glutamic acid. “Insertions” or “deletions” are preferably in the range of about 1 to about 20 amino acids, more preferably 1 to 10 amino acids. The variation allowed may be experimentally determined by systematically making insertions, deletions, or substitutions of amino acids in a polypeptide molecule using recombinant DNA techniques and assaying the resulting recombinant variants for activity.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence may occur at the amino or carboxy terminal positions of the reference amino acid sequence or anywhere between those terminal positions, interspersed either individually among residues in the reference sequence or in one or more contiguous groups within the references sequence.

As a practical matter, whether any particular polypeptide is at least 80%, 85%, 90%, 95%, 96,%, 97%, 98%, or 99% identical to a reference polypeptide can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al., Comp. Appl. Biosci. 6:237-245 (1990). In a sequence alignment the query and subject sequences are either both nucleotide sequences or both amino acid sequences. The result of said global sequence alignment is in percent identity. Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty-0.05, Window Size=500 or the length of the subject amino acid sequence, whichever is shorter.

If the subject sequence is shorter than the query sequence due to N- or C-terminal deletions, not because of internal deletions, a manual correction must be made to the results. This is because the FASTDB program does not account for N- and C-terminal truncations of the subject sequence when calculating global percent identity. For subject sequences truncated at the N- and C-termini; relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence.

For example, a 90 amino acid residue subject sequence is aligned with a 100 residue query sequence to determine percent identity. The deletion occurs at the N-terminus of the subject sequence and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence which are not matched/aligned with the query. In this case, the percent identity calculated by FASTDB is not manually corrected. Once again, only residue positions outside the N- and C-terminal ends of the subject sequence, as displayed in the FASTDB alignment, which are not matched/aligned with the query sequence are manually corrected for: No other manual corrections are to be made for the purposes of the present invention.

Polypeptides of the invention include those that are at least about 80%, 85%, 90%, 95%, 96%, 97%, 98%, 99%, or 100% identical to the amino acid sequences of SEQ ID Nos: 1-11 and 13-46, including functional fragments or variants thereof. The invention encompasses polypeptides comprising the amino acid sequences of any of SEQ ID Nos: 1-11 and 13-46, with conservative amino acid substitutions. Other embodiments of the invention include RRM3-derived peptides wherein the RRM3-derived peptide of CAPERα does not include the 5′ β-sheet of the RRM3 domain of CAPERα. In another embodiment, the RRM3-derived peptide of CAPERα does not include the 5′ α-helix of the RRM3 domain of CAPERα.

In some embodiments, the polypeptide of the invention is conjugated to a protein transduction domain. In some embodiments, the polypeptide of the invention is conjugated to a cell penetrating peptide. In specific embodiments, the cell penetrating peptide is Penetratin. In further embodiments, the cell penetrating peptide amino acid sequence is SEQ ID NO: 12, or variants thereof. In other embodiments, the polypeptide of the invention is conjugated to a tag, e.g., polyhistidine, myc, FLAG, GST, V5, myc, etc. In other embodiments, the polypeptide of the invention is conjugated to polymers, e.g., PEG and other polyethers. In other embodiments, the polypeptide of the invention is conjugated to lipids, e.g., myristoylation, acetylation, palmitoylation, etc.

The polypeptides of the invention may be encoded by a single polynucleotide. RRM3-derived peptides of the present invention and fragments thereof are generally encoded by polynucleotides. The term “polynucleotide” is intended to encompass a singular nucleic acid as well as plural nucleic acids, and refers to an isolated nucleic acid molecule or construct, e.g., messenger RNA (mRNA), virally-derived RNA, or plasmid DNA (pDNA). A polynucleotide may comprise a conventional phosphodiester bond or a non-conventional bond (e.g., an amide bond, such as found in peptide nucleic acids (PNA)). The term “nucleic acid” refers to any one or more nucleic acid segments, e.g., DNA or RNA fragments, present in a polynucleotide. By “isolated” nucleic acid or polynucleotide is intended a nucleic acid molecule, DNA or RNA, which has been removed from its native environment. For example, a recombinant polynucleotide encoding a therapeutic polypeptide contained in a vector is considered isolated for the purposes of the present invention. Further examples of an isolated polynucleotide include recombinant polynucleotides maintained in heterologous host cells or purified (partially or substantially) polynucleotides in solution. Isolated RNA molecules include in vivo or in vitro RNA transcripts of the present invention, as well as positive and negative strand forms, and double-stranded forms, of pestivirus vectors disclosed herein.

Isolated polynucleotides or nucleic acids according to the present invention further include such molecules produced synthetically. In addition, a polynucleotide or a nucleic acid may be or may include a regulatory element such as a promoter, ribosome binding site, or a transcription terminator.

As used herein, a “coding region” is a portion of nucleic acid which consists of codons translated into amino acids. Although a “stop codon” (TAG, TGA, or TAA) is not translated into an amino acid, it may be considered to be part of a coding region, if present, but any flanking sequences, for example promoters, ribosome binding sites, transcriptional terminators, introns, 5′ and 3′ non-translated regions, and the like, are not part of a coding region. Two or more coding regions of the present invention can be present in a single polynucleotide construct, e.g., on a single vector. Furthermore, any vector may contain a single coding region, or may comprise two or more coding regions, e.g., a vector of the present invention may encode one or more polyproteins. In addition, a vector, polynucleotide, or nucleic acid of the invention may encode heterologous coding regions, either fused or unfused to a first or second nucleic acid encoding the RRM3-derived peptide of the invention, or variant or derivative thereof. Heterologous coding regions include without limitation specialized elements or motifs, such as a secretory signal peptide or a heterologous functional domain.

In certain embodiments, the polynucleotide or nucleic acid is DNA. In the case of DNA, a polynucleotide comprising a nucleic acid, which encodes a polypeptide normally may include a promoter and/or other transcription or translation control elements operably associated with one or more coding regions. An operable association is when a coding region for a gene product, e.g., a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription. Suitable promoters and other transcription control regions are disclosed herein.

A variety of transcription control regions are known to those skilled in the art. These include, without limitation, transcription control regions, which function in vertebrate cells, such as, but not limited to, promoter and enhancer segments from cytomegaloviruses (e.g., the immediate early promoter, in conjunction with intron-A), simian virus 40 (e.g., the early promoter), and retroviruses (such as, e.g., Rous sarcoma virus). Other transcription control regions include those derived from vertebrate genes such as actin, heat shock protein, bovine growth hormone and rabbit B-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Similarly, a variety of translation control elements are known to those of ordinary skill in the art. These include, but are not limited to ribosome binding sites, translation initiation and termination codons, and elements derived from viral systems (particularly an internal ribosome entry site, or IRES, also referred to as a CITE sequence).

In other embodiments, a polynucleotide of the present invention is RNA, for example, in the form of messenger RNA (mRNA). RNA of the present invention may be single stranded or double stranded.

Polynucleotide and nucleic acid coding regions of the present invention may be associated with additional coding regions which encode secretory or signal peptides, which direct the secretion of a polypeptide encoded by a polynucleotide of the present invention. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the complete or “full length” polypeptide to produce a secreted or “mature” form of the polypeptide. In certain embodiments, the native signal peptide is used, or a functional derivative of that sequence that retains the ability to direct the secretion of the polypeptide that is operably associated with it. Alternatively, a heterologous mammalian signal peptide, or a functional derivative thereof, may be used. For example, the wild-type leader sequence may be substituted with the leader sequence of human tissue plasminogen activator (TPA) or mouse β-glucuronidase.

The term “expression cassette” refers to a polynucleotide generated recombinantly or synthetically, with a series of specified nucleic acid elements that permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In one embodiment, the expression cassette of the invention comprises polynucleotide sequences that encode RRM3-derived peptides of the invention or fragments thereof.

The term “expression vector” is synonymous with “expression construct” and refers to a DNA molecule that is used to introduce and direct the expression of a specific gene to which it is operably associated into a target cell. The expression vector of the present invention comprises an expression cassette. Expression vectors allow transcription of large amounts of stable mRNA. Once the expression vector is inside the target cell, the ribonucleic acid molecule or protein that is encoded by the gene is produced by the cellular transcription and/or translation machinery. In one embodiment, the expression vector of the invention comprises an expression cassette comprises polynucleotide sequences that encode RRM3-derived peptides of the invention or fragments thereof.

The term “artificial” refers to a synthetic, or non-host cell derived composition, e.g., a chemically-synthesized oligonucleotide.

By a nucleic acid or polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence.

As a practical matter, whether any particular nucleic acid molecule or polypeptide is at least 80%, 85%, 90%, 95%, 96%, 97%, 98%, or 99% identical to a nucleotide sequence or polypeptide sequence of the present invention can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al., Comp. Appl. Biosci. 6:237-245 (1990). In a sequence alignment the query and subject sequences are both DNA sequences. An RNA sequence can be compared by converting U's to T's. The result of said global sequence alignment is in percent identity. Preferred parameters used in a FASTDB alignment of DNA sequences to calculate percent identity are: Matrix=Unitary, k-tuple=4, Mismatch Penalty=1, Joining Penalty-30, Randomization Group Length=0, Cutoff Score=1, Gap Penalty=5, Gap Size Penalty=0.05, Window Size=500 or the length of the subject nucleotide sequences, whichever is shorter.

If the subject sequence is shorter than the query sequence because of 5′ or 3′ deletions, not because of internal deletions, a manual correction must be made to the results. This is because the FASTDB program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by results of the FASTDB sequence alignment. This percentage is then subtracted from the percent identity, calculated by the above FASTDB program using the specified parameters, to arrive at a final percent identity score. This corrected score is what is used for the purposes of the present invention. Only bases outside the 5′ and 3′ bases of the subject sequence, as displayed by the FASTDB alignment, which are not matched/aligned with the query sequence, are calculated for the purposes of manually adjusting the percent identity score.

For example, a 90 base subject sequence is aligned to a 100 base query sequence to determine percent identity. The deletions occur at the 5′ end of the subject sequence and therefore, the FASTDB alignment does not show a matched/alignment of the first 10 bases at 5′ end. The 10 unpaired bases represent 10% of the sequence (number of bases at the 5′ and 3′ ends not matched/total number of bases in the query sequence) so 10% is subtracted from the percent identity score calculated by the FASTDB program. If the remaining 90 bases were perfectly matched the final percent identity would be 90%. In another example, a 90 base subject sequence is compared with a 100 base query sequence. This time the deletions are internal deletions so that there are no bases on the 5′ or 3′ of the subject sequence which are not matched/aligned with the query. In this case the percent identity calculated by FASTDB is not manually corrected. Once again, only bases 5′ and 3′ of the subject sequence which are not matched/aligned with the query sequence are manually corrected for. No other manual corrections are to be made for the purposes of the present invention.

The invention also encompasses an isolated nucleic acid encoding an RRM3-derived peptide of the invention or fragment thereof, wherein the nucleic acid comprises a sequence that encodes the amino acid sequence of any of SEQ ID Nos: 1-11 and 13-46. A further embodiment includes an isolated nucleic acid that encodes the amino acid sequence of any of SEQ ID Nos: 1-11 and 13-46, with conservative amino acid substitutions. The polynucleotides may be expressed as a single polynucleotide that encodes the entire RRM3-derived peptide.

### Host Cells

As used herein, the term “host cell” refers to any kind of cellular system which can be engineered to generate the RRM3-derived peptides of the invention or fragments thereof. In one embodiment, the host cell is engineered to allow the production of an RRM3-derived peptide fragment. Host cells include cultured cells, e.g., mammalian cultured cells, such as CHO cells, HEK, BHK cells, NSO cells, Sp2/0 cells, YO myeloma cells, P3X63 mouse myeloma cells, PER cells, PER.C6 cells or hybridoma cells, yeast cells, insect cells, bacterial cells and plant cells, to name only a few, but also cells comprised within a transgenic animal, transgenic plant or cultured plant or animal tissue. In one embodiment, the host cell of the invention comprises an expression vector comprising polynucleotide sequences that encode RRM3-derived peptides of the invention or fragments thereof. Host cells of the invention may be eukaryotic or prokaryotic.

### Purification of RRM3-Derived Peptides and Fragments Thereof

The RRM3-derived peptides of the invention or fragments thereof can be purified by art-known techniques such as high performance liquid chromatography, ion exchange chromatography, gel electrophoresis, affinity chromatography, size exclusion chromatography, and the like. The actual conditions used to purify a particular protein will depend, in part, on factors such as net charge, hydrophobicity, hydrophilicity, etc., and will be apparent to those having skill in the art.

For affinity chromatography purification, any antibody which specifically binds the RRM3-derived peptide may be used. For the production of antibodies, various host animals, including, but not limited to rabbits, mice, rats, etc., may be immunized by injection with a RRM3-derived peptide of the invention or a fragment thereof. The RRM3-derived peptide may be attached to a suitable carrier, such as bovine serum albumin (BSA), by means of a side chain functional group or linkers attached to a side chain functional group. Various adjuvants may be used to increase the immunological response, depending on the host species, including but not limited to, Freund's (complete and incomplete), mineral gels such as aluminum hydroxide, surface active substances such as lysolecithin, pluronic polyols, polyanions, peptides, oil emulsions, keyhold limpet hemocyanin, dinitrophenol, and potentially useful human adjuvants such as BCG (bacilli Calmette-Guerin) and Corynebacterium parvum. Accordingly, one embodiment includes a method for producing the RRM3-derived peptides of the invention by culturing a host cell comprising an expression vector comprising polynucleotide sequences that encode RRM3-derived peptides of the invention or fragments thereof under conditions suitable for the expression of the same.

### Methods of Using RRM3-Derived Peptides

The RRM3-derived peptides of the invention are useful for disrupting CAP/MLL1 complexes and gene expression in cancer cells, thereby influencing cancer cell proliferation and cell death. The RRM3-derived peptide of the invention is also useful as a diagnostic reagent. The suitability of using an RRM3-derived peptide for cancer treatment can be determined by the disruption of CAP/MLL1 complexes in cancer cells obtained from patient biopsies.

In some embodiments, an effective amount of the RRM3-derived peptides of the invention are administered to a cell. In other embodiments, a therapeutically effective amount of the RRM3-derived peptide of the invention is administered to an individual for the treatment of disease. The term “effective amount” as used herein is defined as the amount of the RRM3-derived peptide of the invention that is necessary to result in a physiological change in the cell or tissue to which it is administered. The term “therapeutically effective amount” as used herein is defined as the amount of the RRM3-derived peptide of the invention that eliminates, decreases, delays, minimizes or prevents adverse effects of a disease.

The RRM3-derived peptides of the invention may be administered to a subject per se or in the form of a pharmaceutical composition. In one embodiment, the disease is a proliferative disorder, such as cancer. Non-limiting examples of proliferative disorders such as cancers include breast cancer, brain cancer, lung cancer, bladder cancer, prostate cancer, fibrosarcoma, ovarian cancer, thyroid cancer, liver cancer, leukemia, and colorectal cancer. In a specific embodiment, the breast cancer is estrogen receptor positive (ER+). In a further embodiment, the breast cancer is human epidermal growth factor receptor 2 positive (HER+). In yet a further embodiment, the breast cancer is triple-negative breast cancer (TNBC). In particular embodiments, the RRM3-derived peptide of CAPERα is nontoxic to noncancerous cells. Other cell proliferation disorders that can be treated using an RRM3-derived peptide of the present invention include, but are not limited to neoplasms located in the: breast, brain, lungs, bladder, prostate, bone, ovary, thyroid, liver, and skin. Also included are pre-cancerous conditions or lesions and cancer metastases. Similarly, other cell proliferation disorders can also be treated by the RRM3-derived peptides of the present invention. A skilled artisan readily recognizes that in some cases the RRM3-derived peptides may not provide a cure but may only provide partial benefit. In some embodiments, a physiological change having some benefit is also considered therapeutically beneficial. Thus, in some embodiments, an amount of RRM3-derived peptide that provides a physiological change is considered an “effective amount” or a “therapeutically effective amount.”

In some embodiments of the invention, the administration of a therapeutically effective amount of the RRM3-derived peptide of CAPERα disrupts the CAPERα/mixed lineage leukemia 1 (MLL1) (CAP/MLL1) complex. In some embodiments, the administration of a therapeutically effective amount of the RRM3-derived peptide of CAPERα reduces in vivo interaction between CAPERα and MLL1 by about 5%, 10%, 15%, 20%, 25%, 30%, 35%, 40%, 45%, 50%, 55%, 60%, 65%, 70%, 75%, 80%, 85%, 90%, 95%, or 100%. In some embodiments, the administration of a therapeutically effective amount of the RRM3-derived peptide of CAPERα regulates chromatin marks. In other embodiments, the administration of a therapeutically effective amount of the RRM3-derived peptide of CAPERα prevents MLL1 occupancy in the CAP/MLL1 complex.

In other embodiments, the administration of a therapeutically effective amount of the RRM3-derived peptide of CAPERα inhibits the catalytic activity of the CAP/MLL1 complex. In some embodiments, the administration of a therapeutically effective amount of the RRM3-derived peptide of CAPERα inhibits the catalytic activity of the CAP/MLL1 complex by about 5%, 10%, 15%, 20%, 25%, 30%, 35%, 40%, 45%, 50%, 55%, 60%, 65%, 70%, 75%, 80%, 85%, 90%, 95%, or 100%.

In other embodiments, the RRM3-derived peptide of CAPERα disrupts binding to the CAP/MLL1 complex by ASH2L. In some embodiments, the administration of a therapeutically effective amount of the RRM3-derived peptide of CAPERα reduces in vivo interaction between the CAP/MLL1 complex and ASH2L by about 5%, 10%, 15%, 20%, 25%, 30%, 35%, 40%, 45%, 50%, 55%, 60%, 65%, 70%, 75%, 80%, 85%, 90%, 95%, or 100%.

In other embodiments, the RRM3-derived peptide of CAPERα inhibits histone 3, lysine 4 (H3K4) trimethylation of target genes. In some embodiments, the administration of a therapeutically effective amount of the RRM3-derived peptide of CAPERα inhibits H3K4 trimethylation of target genes by about 5%, 10%, 15%, 20%, 25%, 30%, 35%, 40%, 45%, 50%, 55%, 60%, 65%, 70%, 75%, 80%, 85%, 90%, 95%, or 100%.

In other embodiments, the RRM3-derived peptide of CAPERα inhibits histone 3, lysine 9 (H3K9) acetylation of target genes. In some embodiments, the administration of a therapeutically effective amount of the RRM3-derived peptide of CAPERα inhibits H3K9 acetylation of target genes by about 5%, 10%, 15%, 20%, 25%, 30%, 35%, 40%, 45%, 50%, 55%, 60%, 65%, 70%, 75%, 80%, 85%, 90%, 95%, or 100%.

In other embodiments, the RRM3-derived peptide of CAPERα increases expression of one or more of the following proteins: NFKβ, TNF, and interferon. In some embodiments, the administration of a therapeutically effective amount of the RRM3-derived peptide of CAPERα increases expression of one or more of NFKβ, TNF, and/or interferon by about 5%, 10%, 15%, 20%, 25%, 30%, 35%, 40%, 45%, 50%, 55%, 60%, 65%, 70%, 75%, 80%, 85%, 90%, 95%, or 100%.

In some embodiments, the RRM3-derived peptide of CAPERα results in decreases proliferation of cancer cells by about 30%. In some embodiments, the administration of a therapeutically effective amount of the RRM3-derived peptide of CAPERα decreases cancer cell proliferation by about 5%, 10%, 15%, 20%, 25%, 30%, 35%, 40%, 45%, 50%, 55%, 60%, 65%, 70%, 75%, 80%, 85%, 90%, 95%, or 100%.

In other embodiments, the RRM3-derived peptide of CAPERα results in increases cell death by about 20%. In some embodiments, the administration of a therapeutically effective amount of the RRM3-derived peptide of CAPERα increases cell death by about 5%, 10%, 15%, 20%, 25%, 30%, 35%, 40%, 45%, 50%, 55%, 60%, 65%, 70%, 75%, 80%, 85%, 90%, 95%, or 100%.

In some embodiments, the RRM3-derived peptide of CAPERα regulates transcription one or more of the following genes: BCL2L1, BCL6, JUNB, CCND1, FOXA1, FOXM1, ESR1, MYC, and GATA3. In other embodiments, the RRM3-derived peptide of CAPERα regulates transcription of one or more of the genes listed in Table 1. In yet further embodiments, the RRM3-derived peptide of CAPERα regulates transcription of one or more of the genes listed in Table 2. In yet further embodiments, the RRM3-derived peptide of CAPERα regulates transcription of breast cancer pioneer genes. In some embodiments, the RRM3-derived peptide of CAPERα upregulates the transcription of one or more of the following genes: CASPASE gene 2, CASPASE gene 3, CASPASE gene 4, CASPASE gene 6, CASPASE gene 8, CASPASE gene 9, KMT2B, KMT2C, KMT2D, SETD1A, vREL, and SETD1B.

Lengthy table referenced here
US20220265769A1-20220825-T00001
Please refer to the end of the specification for access instructions.

Lengthy table referenced here
US20220265769A1-20220825-T00002
Please refer to the end of the specification for access instructions.

Lengthy table referenced here
US20220265769A1-20220825-T00003
Please refer to the end of the specification for access instructions.

In some embodiments, the RRM3-derived peptide of CAPERα inhibits CAP/MLL1 complex binding to chromatin bearing one or more of the following MYC, Rb, E2F1, STAT1/3, ARNT, and GABPB1/2. In some embodiments, the administration of a therapeutically effective amount of the RRM3-derived peptide of CAPERα inhibits CAP/MLL1 complex binding to chromatin bearing one or more of the following MYC, Rb, E2F1, STAT1/3, ARNT, and GABPB1/2, by about 5%, 10%, 15%, 20%, 25%, 30%, 35%, 40%, 45%, 50%, 55%, 60%, 65%, 70%, 75%, 80%, 85%, 90%, 95%, or 100%.

In other embodiments, the RRM3-derived peptide of CAPERα decreases association of MLL1, ASH2L, RbBP5, and/or WDR5 with genomic promoters. In some embodiments, the administration of a therapeutically effective amount of the RRM3-derived peptide of CAPERα decreases association of MLL1, ASH2L, RbBP5, and/or WDR5 with genomic promoters, by about 5%, 10%, 15%, 20%, 25%, 30%, 35%, 40%, 45%, 50%, 55%, 60%, 65%, 70%, 75%, 80%, 85%, 90%, 95%, or 100%.

The subject, patient, or individual in need of treatment is typically a mammal, more specifically a human.

### Compositions, Formulations, Dosages, and Routes of Administration

Pharmaceutical compositions of the present invention comprise an effective amount of one or more RRM3-derived peptides dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that contains at least one RRM3-derived peptide and optionally an additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards or corresponding authorities in other countries.

As used herein, “pharmaceutically acceptable carrier” includes any and all solvents, buffers, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, e.g., Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The RRM3-derived peptides may comprise different types of carriers depending on whether it is to be administered in solid, liquid or aerosol form, and whether it need to be sterile for such routes of administration as injection. The present invention can be administered intravenously, intradermally, intraarterially, intraperitoneally, intralesionally, intracranially, intraarticularly, intraprostatically, intrasplenically, intrarenally, intrapleurally, intratracheally, intranasally, intravitreally, intravaginally, intrarectally, topically, intratumorally, intramuscularly, intraperitoneally, subcutaneously, subconjunctivally, intravesicularlly, mucosally, intrapericardially, intraumbilically, intraocularally, orally, topically, locally, inhalation (e.g. aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, e.g., Remington's Pharmaceutical Sciences, 18th Ed. Mack Printing Company, 1990, incorporated herein by reference). Parenteral administration, in particular intravenous injection, is most commonly used for administering polypeptide molecules such as the RRM3-derived peptides of the invention.

The actual dosage amount of a composition of the present invention administered to a subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of the RRM3-derived peptide of the invention. In other embodiments, the RRM3-derived peptides may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The RRM3-derived peptides may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof, and/or buffering agents to maintain physiologically acceptable pH values.

In other embodiments, one may use eye drops, nasal solutions or sprays, aerosols or inhalants in the present invention. Such compositions are generally designed to be compatible with the target tissue type. In a non-limiting example, nasal solutions are usually aqueous solutions designed to be administered to the nasal passages in drops or sprays. Nasal solutions are prepared so that they are similar in many respects to nasal secretions, so that normal ciliary action is maintained. Thus, in some embodiments the aqueous nasal solutions usually are isotonic or slightly buffered to maintain a pH of about 5.5 to about 6.5. In addition, antimicrobial preservatives, similar to those used in ophthalmic preparations, drugs, or appropriate drug stabilizers, if required, may be included in the formulation. For example, various commercial nasal preparations are known and include drugs such as antibiotics or antihistamines.

In certain embodiments, the RRM3-derived peptide is prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. Preferred carriers for oral administration comprise inert diluents, assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

In certain embodiments, an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

Additional formulations which are suitable for other modes of administration include suppositories. Suppositories are solid dosage forms of various weights and shapes, usually medicated, for insertion into the rectum, vagina or urethra. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

Sterile injectable solutions are prepared by incorporating the RRM3-derived peptides of the invention in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less than 0.5 ng/mg protein.

In particular embodiments, prolonged absorption of an injectable composition can be brought about by the use in the compositions of agents delaying absorption, such as, for example, aluminum monostearate, gelatin or combinations thereof.

Pharmaceutical compositions comprising the RRM3-derived peptides of the invention may be manufactured by means of conventional mixing, dissolving, granulating, dragee-making, levigating, emulsifying, encapsulating, entrapping or lyophilizing processes. Pharmaceutical compositions may be formulated in conventional manner using one or more physiologically acceptable carriers, diluents, excipients or auxiliaries which facilitate processing of the proteins into preparations that can be used pharmaceutically. Proper formulation is dependent upon the route of administration chosen.

For topical administration the RRM3-derived peptides of the invention may be formulated as solutions, gels, ointments, creams, suspensions, etc. as are well-known in the art.

Systemic formulations include those designed for administration by injection, e.g. subcutaneous, intravenous, intramuscular, intrathecal or intraperitoneal injection, as well as those designed for transdermal, transmucosal, inhalation, oral or pulmonary administration.

For injection, the RRM3-derived peptides of the invention may be formulated in aqueous solutions, preferably in physiologically compatible buffers such as Hanks' solution, Ringer's solution, or physiological saline buffer. The solution may contain formulatory agents such as suspending, stabilizing and/or dispersing agents.

Alternatively, the RRM3-derived peptides may be in powder form for constitution with a suitable vehicle, e.g., sterile pyrogen-free water, before use.

For transmucosal administration, penetrants appropriate to the barrier to be permeated are used in the formulation. Such penetrants are generally known in the art.

For oral administration, the RRM3-derived peptides can be readily formulated by combining the RRM3-derived peptides with pharmaceutically acceptable carriers well known in the art. Such carriers enable the RRM3-derived peptides of the invention to be formulated as tablets, pills, dragees, capsules, liquids, gels, syrups, slurries, suspensions and the like, for oral ingestion by a patient to be treated. For oral solid formulations such as, for example, powders, capsules and tablets, suitable excipients include fillers such as sugars, e.g. lactose, sucrose, mannitol and sorbitol; cellulose preparations such as maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP); granulating agents; and binding agents. If desired, disintegrating agents may be added, such as the cross-linked polyvinylpyrrolidone, agar, or alginic acid or a salt thereof such as sodium alginate.

If desired, solid dosage forms may be sugar-coated or enteric-coated using standard techniques.

For oral liquid preparations such as, for example, suspensions, elixirs and solutions, suitable carriers, excipients or diluents include water, glycols, oils, alcohols, etc. Additionally, flavoring agents, preservatives, coloring agents and the like may be added.

For buccal administration, the RRM3-derived peptides may take the form of tablets, lozenges, etc. formulated in conventional manner.

For administration by inhalation, the RRM3-derived peptides for use according to the invention are conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of gelatin for use in an inhaler or insufflator may be formulated containing a powder mix of the RRM3-derived peptide and a suitable powder base such as lactose or starch.

The RRM3-derived peptides may also be formulated in rectal or vaginal compositions such as suppositories or retention enemas, e.g., containing conventional suppository bases such as cocoa butter or other glycerides.

In addition to the formulations described previously, the RRM3-derived peptides may also be formulated as a depot preparation. Such long acting formulations may be administered by implantation (for example subcutaneously or intramuscularly) or by intramuscular injection. Thus, for example, the RRM3-derived peptides may be formulated with suitable polymeric or hydrophobic materials (for example as an emulsion in an acceptable oil) or ion exchange resins, or as sparingly soluble derivatives, for example, as a sparingly soluble salt.

Alternatively, other pharmaceutical delivery systems may be employed. Liposomes and emulsions are well known examples of delivery vehicles that may be used to deliver RRM3-derived peptides of the invention. Certain organic solvents such as dimethylsulfoxide also may be employed, although usually at the cost of greater toxicity. Additionally, the RRM3-derived peptides may be delivered using a sustained-release system, such as semipermeable matrices of solid polymers containing the therapeutic agent. Various of sustained-release materials have been established and are well known by those skilled in the art. Sustained-release capsules may, depending on their chemical nature, release the RRM3-derived peptides for a few weeks up to over 100 days. Depending on the chemical nature and the biological stability of the RRM3-derived peptides, additional strategies for RRM3-derived peptides stabilization may be employed.

As the RRM3-derived peptides of the invention may contain charged side chains or termini, they may be included in any of the above-described formulations as the free acids or bases or as pharmaceutically acceptable salts. Pharmaceutically acceptable salts are those salts which substantially retain the biologic activity of the free bases and which are prepared by reaction with inorganic acids. Pharmaceutical salts tend to be more soluble in aqueous and other protic solvents than are the corresponding free base forms.

The RRM3-derived peptides of the invention will generally be used in an amount effective to achieve the intended purpose. For use to treat or prevent a disease condition, the RRM3-derived peptides of the invention, or pharmaceutical compositions thereof, are administered or applied in a therapeutically effective amount. A therapeutically effective amount is an amount effective to ameliorate or prevent the symptoms, or prolong the survival of, the patient being treated. Determination of a therapeutically effective amount is well within the capabilities of those skilled in the art, especially in light of the detailed disclosure provided herein.

For systemic administration, a therapeutically effective dose can be estimated initially from in vitro assays. For example, a dose can be formulated in animal models to achieve a circulating concentration range that includes the IC₅₀ as determined in cell culture. Such information can be used to more accurately determine useful doses in humans.

Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the RRM3-derived peptides which are sufficient to maintain therapeutic effect. Usual patient dosages for administration by injection range from about 0.1 to 50 mg/kg/day, typically from about 0.5 to 1 mg/kg/day. Therapeutically effective serum levels may be achieved by administering multiple doses each day.

In cases of local administration or selective uptake, the effective local concentration of the RRM3-derived peptides may not be related to plasma concentration. One having skill in the art will be able to optimize therapeutically effective local dosages without undue experimentation.

The amount of RRM3-derived peptide administered will, of course, be dependent on the subject being treated, on the subject's weight, the severity of the affliction, the manner of administration and the judgment of the prescribing physician.

The therapy may be repeated intermittently while symptoms detectable or even when they are not detectable. The therapy may be provided alone or in combination with other drugs. In the case of autoimmune disorders, the drugs that may be used in combination with RRM3-derived peptides of the invention include, but are not limited to, steroid and non-steroid anti-inflammatory agents.

### Toxicity

A therapeutically effective dose of the RRM3-derived peptides described herein will generally provide therapeutic benefit without causing substantial toxicity. Toxicity of the RRM3-derived peptides can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. In one embodiment, the RRM3-derived peptide exhibits a high therapeutic index. The data obtained from these cell culture assays and animal studies can be used in formulating a dosage range that is not toxic, for example, for use in human. The dosage of the RRM3-derived peptides described herein lies preferably within a range of circulating concentrations that include the effective dose with little or no toxicity. The dosage may vary within this range depending upon the dosage form employed and the route of administration utilized. The exact formulation, route of administration and dosage can be chosen by the individual physician in view of the patient's condition. (See, e.g., Fingl et al., 1975, In: The Pharmacological Basis of Therapeutics, Ch. 1, p. 1) (incorporated herein by reference in its entirety.

### Other Agents and Treatments

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adhesion, or agents that increase the sensitivity of the hyperproliferative cells to apoptotic inducers. Immunomodulatory agents include tumor necrosis factor; interferon alpha, beta, and gamma; IL-2 and other cytokines; F42K and other cytokine analogs; or MIP-1, MIP-1β, MCP-1, RANTES, and other chemokines. It is further contemplated that the upregulation of cell surface receptors or their ligands such as Fas/Fas ligand, DR4 or DR5/TRAIL would potentiate the apoptotic inducing abilities of the present invention by establishment of an autocrine or paracrine effect on hyperproliferative cells. Increases in intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyperproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody C225, could be used in combination with the present invention to improve the treatment efficacy.

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

The RRM3-derived peptides of the invention may also be administered in conjunction with chemotherapy, radiation therapy or other immunotherapies. Anti-cancer agents for such combination therapy may, e.g., be selected from the groups of microtubule disruptors (e.g. vinca alkaloids such as vinblastine or vincristine, taxanes such as docetaxel or paclitaxel, epothilones such as ixabepilone), antimetabolites (e.g. anti-folates such as methotrexate or aminopterin, anti-purines such as fludarabine, 6-mercaptopurine or 6-thioguanine, anti-pyrimidines such as 5-fluorouracil, capecitabine or gemcitabine, hydroxyurea), topoisomerase inhibitors (e.g. camptothecin, irinotecan, topotecan, or podophyllotoxins such as etoposide), DNA intercalators (e.g. doxorubicin, daunorubicin, actinomycin, bleomycin), alkylating agents (e.g. cyclophosphamide, chlorambucil, nitrosureas such as carmustine or nimustine, streptozocin, busulfan, cisplatin, oxaliplatin, triethylenemelamine, dacarbazine), hormonal therapies (e.g. glucocorticoids, aromatase inhibitors such as tamoxifene, antiandrogens such as flutamide, gonadotropin-releasing hormone (GnRH) analogs such as leuprolide), antibiotics, kinase inhibitors (e.g. erlotinib, gefitinib, imatinib), receptor antagonists (e.g. antibodies targeting cell surface receptors known to promote carcinogenesis and tumor growth), enzyme inhibitors (e.g. cyclin-dependent kinase (CDK) inhibitors), amino acid-depleting enzymes (e.g. asparaginase), leucovorin, retinoids, activators of tumor cell apoptosis, and antiangiogenic agents.

The following are examples of methods and compositions of the invention. It is understood that various embodiments may be practiced, given the general description provided above.

### EXAMPLES

### Example 1

The following methods and reagents were used in conducting the investigations described in Examples 2-12.

Cell Culture: Cell lines were obtained from ATCC and cultured as recommended. Protein extraction and immunoprecipitations (IPs): IP's were performed as previously described (Kumar P P, et al., eLife. e02805 (2014)). Input lanes contain 5% of protein lysate used for IP; 25% of IP'd material was loaded and subjected to immunoblotting.

Cell Lines: T47D, MCF7, HCC1428, ZR7530, MDA-MB361, MDA-MB453, HCC1954, HCC2157, MDA-MB468, DU4475, MDA-MB231, HCC1395, HCC38, PHF, PME, MCF10A, HEK293, HeLa, Jurkat, HUV-EC-C, UMUC3, HCT116 were purchased from ATCC. All cells were cultured and maintained as per the ATCC recommended protocols.

Human tissue samples and lysates: Normal human tissue lysates were obtained from Novus biologicals. Brain (NB820-59177), ovary (NB820-59243), liver (NB820-59232), small intestine (NB820-59255), bone marrow (NB820-59283), colon (NB820-59205) and lung (NB820-59239). Buffer composition of all the tissue lysates are similar to the Dignam buffer employed for all other lysates/IPs. Deidentified human breast tumor tissues were collected from the Geisinger Clinic surgical oncology biobank in accordance with IRB regulations. The tissues were flash frozen in liquid nitrogen following surgery and stored in −80° C. until immunoprecipitation.

Cell transfection: For overexpression of myc-tagged constructs, HEK293 cells were transfected with indicated vectors using lipofectamine-3000 reagent (ThermoFisher, L3000001) as per the manufacture's procedure. Transfections of siRNAs in T47D, MDA-MB231, PMEs, PHFs, and Jurkat cells were performed by using Neon transfection system (MPK10025). For the overexpression of vector or RRM3 domain in T47D, X-tremeGENE HP DNA transfection reagent (Roche) was used and the transfection was carried out as per the manufacturer's recommendations. siRNAs were used at the concentration of 100 nM in all the knockdown experiments.

Protein extraction and immunoprecipitations: IP's were performed as per the manufacturer's protocol (ThermoFisher, Dynabeads Co-Immunoprecipitation Kit: 14321D). Briefly, lysates were prepared with Dignam buffer and cleared lysates were incubated for 4 hours at 4° C. with specific antibodies followed by incubation with the pre-equilibrated Dynabeads Protein G (Invitrogen) for 2 hours at 4° C. with. Immunoprecipitated beads were washed three times with lysis buffer and resuspended in 6×SDS-loading buffer. Immunoprecipitated proteins were subjected to SDS-PAGE analysis followed by immunoblotting with specific antibodies. Note: Immunoprecipitation antibodies are listed at top and immunoblot antibodies listed at left for figures.

Antibodies: CAPERα (A300-291A) Bethyl laboratories, R-IgG (Santa Cruz (SC)-2027), m-IgG (Santa Cruz, SC-2025), Flag (Sigma, F3165), myc-tag (Santa Cruz, SC-40), Actin (Santa Cruz, SC-47778), H3K9me3 (Cell Signaling, 9754), H3K4me3 (Cell Signaling, 9751; active motif 39159), H3K27me3 (Cell Signaling, 9733), H3K9ace (Cell Signaling, 9649), H3K36me (Cell Signaling, 4909), H3 (Cell Signaling, 9715), H3K27ace (Abram (ab) 4729), H3K9ace (ab176916), Tubulin (CP-06, Calbiochem), rabbit polyclonal Ki67 (Vectorlabs), TBX3(A303-098A) Bethyl laboratories, Ezh2 (Cell Signaling, 5246), MLL1 (Active Motif, 61296), MLL2 (Novus Biologicals, NBP1-89123), EZH2 (Cell Signaling, 5246), WDR5 (Santa Cruz, SC-393080; Active Motif 61485), RbBP5 (Novus biologicals 600-252), ERα (Santa Cruz, SC-7207), HCF1 (ab 137618), Lamin A/C (Santa Cruz, E-1), HDAC1 (Cell Signaling, 2062), HNRNPC1/C2 (Santa Cruz, SC-32308), HNRNPU (Santa Cruz, SC-32315), PABP (Santa Cruz, SC-166027), SAP 155 (Santa Cruz, SC-514655), U2AF65 (Santa Cruz, SC-53942), DDX3 (Santa Cruz, SC-365768), HNRNPA1 (Santa Cruz, SC-32301), DDX17 (Santa Cruz, SC-130650), HNRNPD (Abram, ab61193), DDX21 (Santa Cruz, SC-376953). The WDR5 and ASH2L antibody were gifts from Danny Reinberg.

Plasmids: Wild type and truncations of CAPERα were generated by PCR amplification and then cloned into the pcDNA4/myc-His vector. Individual domains were cloned into PGEX-6P-1 vector. GST-tagged domains were cloned in pcDNA4/myc-His vector. GST-tagged RRM3 microdeletion constructs were synthesized by IDTDNA technologies as a g-block fragments and cloned in pCDNA4/myc-His vector. Cassettes for generating CPPs peptides were designed as g-blocks by IDTDNA technologies and cloned in to pGEX-6P-1. Correct sequence of all plasmids was confirmed.

Purification of GST-CAPERα proteins: GST-tagged RBM39 full length (FL) and deletion constructs were expressed in BL-21 DE3 cells and induced by 0.25 mM IPTG. Columns of GST and GST-RBM39 FL and deletions were prepared as per the manufacture's protocol (GE17075601). Bound proteins were eluted with one volume of elution buffer (50 mM Tris-HCl, 10 mM reduced Glutathione, pH 8.0) and analyzed by SDS-PAGE. Purified proteins were visualized by Coomassie brilliant blue stain. For the CPPs, the GST tag was removed by PreScission protease (GE Healthcare).

GST pull-down assays: GST-tagged RBM39 full length or individual domains, and GST bound columns were incubated with 5 mg of T47D Dignam lysate at 40° C. overnight. Beads were washed 3 times Dignam buffer C and bound proteins were analyzed by western blotting with the respective antibody.

Myc pull-down assays: Myc pull-down was performed as per manufacturer's protocol (ThermoFisher: Pierce c-Myc-Tag IP/Co-IP Kit: 2360).

Electrophoretic mobility shift assays (EMSAs): GST and GST-RBM39 FL and individual domains were purified and incubated with synthetic oligos (IDTDNA technologies) or PCR amplified and purified DNA segments of RBM39-MLL1 targets promoter regions. Protein-DNA binding reactions were resolved by 0.8% agarose gel or 6% native polyacrylamide gel electrophoresis and stained with SYBR GOLD nucleic acid stain and documented using ChemiDoc XRS+ system.

Nuclear and cytoplasmic separations were performed as per the manufacturer's protocol (GenDEPOT: N2100-010).

Retroviral transduction and selection of stable cells were performed as per the published literature (Kumar et al., 2014) (http://delangelab.rockefeller.edu/assets/file/Retrovirusprotocol.pdf). RBM39 shRNAs were generated as per the published literature (Kumar, P. P., et al., eLife, e02805 (2014)) and MLL1 shRNA were prepared from published MLL1 specific siRNAs (Caslini, C., et al., Mol Cell Biol 29:4519-4526 (2009); Hsieh, J. J., et al., Cell 115:293-303 (2003); Yokoyama A, et al., Mol Cell Biol. 24(13):5639-492004).

MLL shRNA FP:
(SEQ ID NO: 48)
GATCCAAGAAGTCAGAGTGCGAAGTCTCAAGAGGACTTCGCACTCTGAC
TTCTTTTTTTTGGA
MLL shRNA RP:
(SEQ ID NO: 49)
AGCTTCCAAAAAAAAGAAGTCAGAGTGCGAAGTCCTCTTGAGACTTCGC
ACTCTGACTTCTTG

Population doubling assay/3T5 growth curves: were performed as in (Lessnick, S. L., et al., Cancer Cell 1:393-401 (2002)). T47D and MCF10A cells were plated in a 10 cm dish and transduced with retrovirus. After 24 hrs, cells were incubated with specific mammalian antibiotic medium (puromycin or blasticidin) for an additional 24-72 hrs. On day 0 of the 3T5 growth curve, cells were trypsinized, counted and 500,000 cells then plated per 10 cm dish. This procedure was repeated every 3 days for 15 days. Population doublings were calculated by (log N1/log 2)−(log N0/log 2) N1=current cell count, N0=Initial cell count. Curves shown in FIG. 5 are representative of 2 independent experiments. Curves shown are representative of 2 independent experiments.

Crystal violet assay/optical density method of cell quantitation: 5×10⁵ cells were plated per well in 6-well tissue culture plates. At times indicated cells were washed with PBS and fixed for 10 minutes in a 10% formalin solution. Cells were then rinsed with distilled water and subsequently stained with 100 μl 0.1% crystal violet solution for 30 min. Stained cells were rinsed with water to remove the excess traces of the staining solution. Cell-associated crystal violet dye was extracted with 500 μl of 10% acetic acid. Aliquots were collected and optical density was measured at 590 nm wavelength. Each point on the curve shown represents 3 independent wells.

RNA isolation and reverse transcription-PCR analysis: Total RNA was prepared using the RNeasy RNA isolation kit (Qiagen) or NucleoSpin RNA II Kit (Clontech) and cDNA was synthesized with cDNA EcoDry Premix Double Primed (Clontech). Q-RT-PCR was performed with So FastEvagreen Supermix (Bio-Rad) as per manufacturer's protocol.

siRNA Sequences:

Control siRNA:
S:
(SEQ ID NO: 50)
AAUUCUCCGAACGUGUCACGUUU
AS:
(SEQ ID NO: 51)
ACGUGACACGUUCGGAGAAUUUU
RBM39 siRNA 1:
S:
(SEQ ID NO: 52)
GACAGAAAUUCAAGACGUUUU
AS:
(SEQ ID NO: 53)
AACGUCUUGAAUUUCUGUCUU
RBM39 siRNA 2:
S:
(SEQ ID NO: 54)
GAAGCGAAGUAGAGACAGAUU
AS:
(SEQ ID NO: 55)
UCUGUCUCUACUUCGCUUCUU
RBM39 siRNA 3:
S:
(SEQ ID NO: 56)
AAGAUUGGGUUGCCUCAUAUUUU
AS:
(SEQ ID NO: 57)
AAUAUGAGGCAACCCAAUCUUUU
siSMART Pool: SiGENOME Human RBM39 (9584) SiRNA
SMART Pool, (Dharmacon, M-011965-00-0010).
MLL1 siRNA 1:
(SEQ ID NO: 58)
AAGAAGUCAGAGUGCGAAGUC
(Caslini et al., 2009)
MLL1 siRNA 2:
(SEQ ID NO: 59)
GGACAAGAGTAGAGAGAGA
(Wang, X., et al., J Cell Sci 125:4058-4066 (2012))
MLL siRNA mix from Santa Cruz (SC-38039)
MLL CRISPR/Cas9 KO Plasmid (SC-401307) +
MLL HDR Plasmid (Santa Cruz, SC-401307)-HDR

RT-PCR Primer Sequences:

CAPERα:
(SEQ ID NO: 60)
CGGAACAGGCGTTTAGAGAA,
(SEQ ID NO: 61)
TGGCACTGCTCAACTTGTTC
MLL1:
(SEQ ID NO: 62)
CAGCTATCCTCTCAGATCCATC,
(SEQ ID NO: 63)
CCTTGTCTTTCCGGACTTTCTG
CCNB1:
(SEQ ID NO: 64)
GGCTTTCTCTGATGTAATTCTTGC,
(SEQ ID NO: 65)
GTATTTTGGTCTGACTGCTTGC
CDK1:
(SEQ ID NO: 66)
TGGATCTGAAGAAATACTTGGATTCTA,
(SEQ ID NO: 67)
CAATCCCCTGTAGGATTTGG
CCNA2:
(SEQ ID NO: 68)
CTGCATTTGGCTGTGAACTAC,
(SEQ ID NO: 69)
ACAAACTCTGCTACTTCTGGG
BCL6:
(SEQ ID NO: 70)
CTGCAGATGGAGCATGTTGT,
(SEQ ID NO: 71)
TCTTCACGAGGAGGCTTGAT
JUNB:
(SEQ ID NO: 72)
GCATGAGGAAACGCATCGCTGCCTCCAAGT,
(SEQ ID NO: 73)
GCGACCAAGTCCTTCCCACTCGTGCACACT
CCND1:
(SEQ ID NO: 74)
AAGGCGGAGGAGACCTGCGCG,
(SEQ ID NO: 75)
ATCGTGCGGCATTGCGGC
GATA3:
(SEQ ID NO: 76)
TGTCTGCAGCCAGGAGAGC,
(SEQ ID NO: 77)
ATGCATCAAACAACTGTGGCCA
HMGA2:
(SEQ ID NO: 78)
GTCCCTCTAAAGCAGCTCAAAA,
(SEQ ID NO: 79)
CTCCCTTCAAAAGATCCAACTG
DUSP6:
(SEQ ID NO: 80)
CCTGAGGCCATTTCTTTCATAGA,
(SEQ ID NO: 81)
GTCACAGTGACTGAGCGGCTAAT
SATB1:
(SEQ ID NO: 82)
GATCTATGAATAAGCCTTTGGAG,
(SEQ ID NO: 83)
TTTCGTCCTGGTATATTCGGT
CBX8:
(SEQ ID NO: 84)
ACAAACCCATGTTTCGAAGG,
(SEQ ID NO: 85)
AGTGTCCAATGGTGGAGGAC
SSRP1:
(SEQ ID NO: 86)
AGGATGAGCTGGCTAGAGAGAC,
(SEQ ID NO: 87)
CCCACCCCACCCCTGTACTAA
LRP5:
(SEQ ID NO: 88)
GGGAGACGCCAAGACAGACAAGATCG,
(SEQ ID NO: 89)
GGTGAAGACCAAGAAGGCCTCAGG
G2E3:
(SEQ ID NO: 90)
CCCTTGGTGTTTTGGAGAAA,
(SEQ ID NO: 91)
GAGTGTTTCTTATGGTGAAGTCCA
ESR1:
((SEQ ID NO: 92)
GGCCAAATTCAGATAATCGAC,
(SEQ ID NO: 93)
CCACTTCGTAGCATTTACGG
HPRT:
(SEQ ID NO: 94)
GCTGGTGAAAAGGACCTCT,
(SEQ ID NO: 95)
CACAGGACTAGAACACCTGC
PCNA:
(SEQ ID NO: 96)
CCATCCTCAAGAAGGTGTTGG,
(SEQ ID NO: 97)
GTGTCCCATATCCGCAATTTTAT
BCL2l1:
(SEQ ID NO: 98)
AAACTGGGTCGCATTGTGG,
(SEQ ID NO: 99)
TCTCGGCTGCTGCATTGTTC
TCF7:
(SEQ ID NO: 100)
TGCAGCTATACCCAGGCTGG,
(SEQ ID NO: 101)
CCTCGACCGCCTCTTCTTC
MMP3:
(SEQ ID NO: 102)
CCTGCTTTGTCCTTTGATGC,
(SEQ ID NO: 103)
TGAGTCAATCCCTGGAAAGTC
TERT:
(SEQ ID NO: 104)
CGGAAGAGTGTCTGGAGCAA,
(SEQ ID NO: 105)
GGATGAAGCGGAGTCTGGA
ATM:
(SEQ ID NO: 106)
TGCTGACAATCATCACCAAGTTC,
(SEQ ID NO: 107)
TCTCCCTTCGTGTCCTGGAA
RAB3A:
(SEQ ID NO: 108)
TGGGTTCGAGTTCTTTGAGG,
(SEQ ID NO: 109)
GTCCAACGACTCGGACATCT
MAPK14:
(SEQ ID NO: 110)
TTCTGTTGATCCCACTTCACTGT,
(SEQ ID NO: 111)
ACACACATGCACACACACTAAC
TGFβ1:
(SEQ ID NO: 112)
AAGGACCTCGGCTGGAAGTG,
(SEQ ID NO: 113)
CCCGGGTTATGCTGGTTGTA
DUSP5:
(SEQ ID NO: 114)
GCTCGCTCAACGTCAACCTCAACTCGGTG,
(SEQ ID NO: 115)
AGTGGCGGCTGCCCTGGTCCAGCACCACC

Chromatin Immunoprecipitation (ChIP): Chromatin was prepared as per manufacturer's protocol (9003S, Cell Signaling).

siRNA knockdown: cells were transfected with control, CAPERα (Kumar et al., 2014) and MLL1 siRNAs (Caslini et al., 2009) using X-treme GENE HP DNA transfection reagent as per manufacturer's instructions. For RNA-sequencing, T47D cells were transfected with two CAPER specific siRNAs (Dowhan D H, et al., Mol Cell. 17(3):429-39 (2005)). For cell studies, a single siRNA was employed as in (Kumar et al., 2014).

Soft agar colony formation assay: To assay for anchorage-independent growth, 5×10⁵ control or CAPERα shRNA transduced T47D or MCF10A cells in 1.5 ml media with 0.35% agarose was applied on top of set 0.5% tissue culture grade agarose in 1.5 ml media in 6 well dishes. Cells were cultured for 14 days, dishes stained with 0.005% crystal violet, and colonies counted.

T47D mouse xenografts: All procedures were approved by the University of Utah IACUC. 10×10⁶ control or CAPERα shRNA transduced T47D cells were suspended in 25 μl Matrigel and implanted into the cleared right inguinal mammary fat pad of 3-week-old NOD/SCID mice as described (Wysocka J, et al., Cell. 121(6):859-72 (2005)). A subcutaneous estrogen pellet was also transplanted behind the shoulder blades. To determine tumor volume, the length and the width were determined by calipers. Tumor volume=½(length×width²).

Cell counts for CPP treatments: Cells were plated in 6-well dishes and incubated with 5 μM concentration of peptides. At days noted, cells were trypsinized and counted using a hemocytometer.

Bioinformatic analyses of RNA and ChIP-Seq data. ChIP-seq Data Standards and Processing Pipeline from ENCODE were employed. Each sample was sequenced with approximately 25M 50 bp reads. Single-end reads were aligned to UCSC HG19 using Burrows-Wheeler Aligner (BWA) (v0.7.15) (Li H, et al., Bioinformatics. 25(14):1754-60 (2009)) and sorted using Samtools (v1.3.1) (Li H, et al., Bioinformatics. 25(16):2078-9 (2009)). Duplicate reads were removed using Picard (v2.9.0) to generate final sorted BAM files. Peaks were called using MACS2 (Zhang Y, et al., Genome Biol. 9(9):R137 (2008)). Overlapped peaks from different individual peaks were merged to generate the final set of ChIP-Seq peaks and investigate the co-occurrency of peaks in different CAPER and MLL samples. To understand the function of these peaks, peaks were annotated to different gene regions according to the following parameters: coding, intronic, 5′ or 3′ UTR, promoter (2 Kb upstream from a transcription start site), upstream (10 Kb upstream from a promoter), downstream (10 Kb downstream from a transcription stop), and intergenic regions. De novo and known motif were then searched for in the ChIP-Seq peaks using HOMER (Heinz S, et al., Mol Cell. 38(4):576-89 (2010)). GREAT (McLean C Y, et al., Nat Biotechnol. 28(5):495-501 (2010)) was used to characterize biological function of target genes including Gene Ontology (GO) (Ashburner M, et al., Nat Genet. 25(1):25-9 (2000)) and MSigDB pathway analysis as described in the results. The function of target genes was annotated based on established lists of housekeeping genes (Eisenberg E, et al., Trends in genetics: TIG. 29(10):569-74 (2013)), oncogenes, and tumor suppressors (Lever J, et al., Nat Methods. 16(6):505-7 (2019)).

For RNA-Seq, raw paired end reads from the FASTQ files were trimmed with Cutadapt https://cutadapt.readthedocs.io/en/stable/to remove the low-quality tails. Reads were aligned on UCSC HG19 using Tophat2 (v2.0.12) (Trapnell C, et al., Nat Protoc. 7(3):562-78 (2012)) guided by RefSeq gene annotation downloaded from UCSC 2/6/19. Cufflinks (v2.2.1) was used to estimate the expression abundances of genes and transcripts. Cuffdiff was used to identify differentially expressed genes (DEGs) between the two replicates of control versus CAPER knockdown, with default parameters and a q-value <0.05 as the significance threshold for all subsequent analyses. DEGs were annotated to the four functional categories mentioned above and investigated the significance of enriched up/downregulated genes by comparing the observed fraction of DEGs among each category against the fraction of DEGs among all genes. The p-value was calculated based on the hypergeometric distribution.

Generation of synthetic peptides from the RRM3 domain: RRM3-derived peptides were commercially synthesized by Abm at purity >80%. Peptides were dissolved in 1λPBS at 1 mg/ml and used at 5 μM in low serum (1%) RPMI media.

MLL1 CRISPR/Cas KO procedure: T47D cells were plated on 6-well plate in complete growth medium (RPMI with 10% FBS). Cells were transfected with MLL CRISPR/Cas9 KO and MLL HDR plasmid at the ratio of 1:1.2 μg. The Neon transfection system was used with a single pulse at 1,700 pulse voltage and 20 ms pulse width. Transfected cells were plated in complete medium for 24 hours followed by puromycin selection. MLL1 knockout efficiency was measured by RT-PCR to assay for transcript levels.

### Example 2

### CAPERα Depletion Reduces Tumorigenesis and Cancer Hallmarks in Breast Cancer Cells

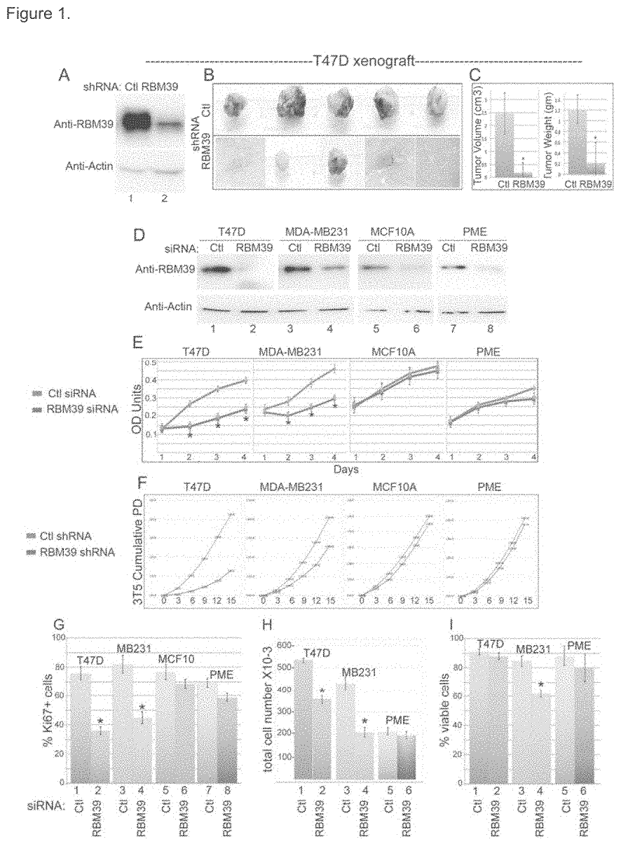

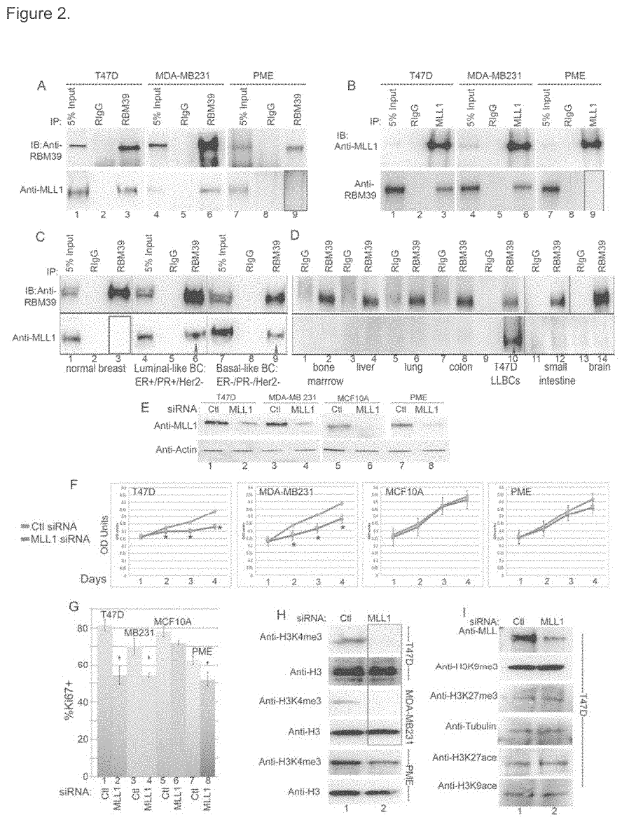

It was previously reported that CAPERα is required to regulate cell proliferation and prevent senescence of primary human fibroblasts, suggesting that CAPERα has oncogenic potential (Kumar et al., 2014). There is evidence that CAPERα plays a role in breast and other cancers (Mercier I, et al., Cell Cycle. 13(8):1256-64 (2014); Mercier I, et al., Am J Pathol. 174(4):1172-90 (2009); Chai Y, et al., Tumour biology: the journal of the International Society for Oncodevelopmental Biology and Medicine. 35(7):6311-7 (2014); Sillars-Hardebol A H, et al., Cellular oncology. 35(4):293-300 (2012); Dutta J, et al., J Virol. 82(21):10792-802 (2008); Huang G, et al., Cancer. 118(8):2106-16 (2012); Dai L, et al., Experimental hematology & oncology. 2(1):15 (2013)). It was investigated herein that CAPERα influences tumorigenesis and invasion in vivo. Cells were xenografted from the T47D ER+, luminal-like breast cancer cell line, transduced with either control or CAPERα shRNA-expressing lentivirus, into the cleared right inguinal mammary fat pad of 3-4 week-old NOD/SCID mice as previously described (DeRose Y S, et al., Current protocols in pharmacology/editorial board, SJ Enna. Chapter 14 (2013)). Effective knockdown of CAPERα protein was verified (FIG. 1A). At 15 weeks, removal of the implantation site revealed that all control xenografts had formed well vascularized tumors (examples shown in FIG. 1B). In marked contrast, 6/8 CAPERα knockdown (kd) xenografts had no tumor, and 2 had tumors that were significantly smaller than controls (examples FIGS. 1B, C).

Whether CAPERα regulated proliferation or survival of T47D and the triple-negative, basal-like breast cancer cell line MDA-MB231, as well as of the non-transformed MCF10A breast epithelial cell line and primary mammary epithelial cells (PMEs) was also investigated. CAPERα knock-down (kd) decreased proliferation of T47D and MDA-MB231 breast cancer cells assayed by crystal violet and population doublings, but did not have this effect in either MCF10A or PME cells (FIGS. 1E, F). Consistent with this, the percentage of Ki67+ cells at 48 hours post kd (FIG. 1G) was also significantly decreased only in breast cancer cells. Although T47D cell viability was unchanged, 20% of MDA-MB231 cells were dead indicating that the altered MDA-MB231 growth curve reflects a combination of decreased proliferation and cell death in response to CAPERα kd (total cell number decreased by 50%, with 20% attributable to cell death, FIGS. 1 H, I). Anchorage-independent cell growth was also decreased (FIG. 11A) as were transcript levels of a sample of cell cycle genes in breast cancer cells, but not benign breast cells (FIG. 11B). Thus, some breast cancer cell hallmarks require CAPERα yet survival and proliferation of normal breast epithelial cells are CAPERα independent.

### Example 3

### Loss of CAPERα Results in Global Changes in Activating Histone Marks

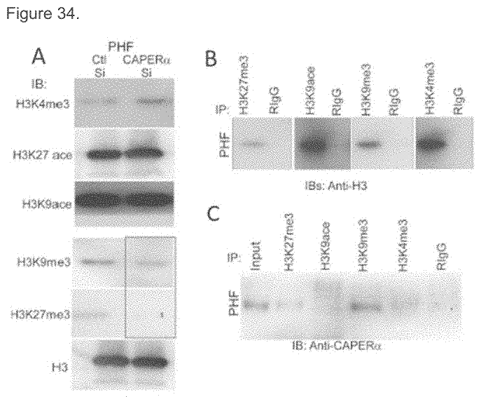

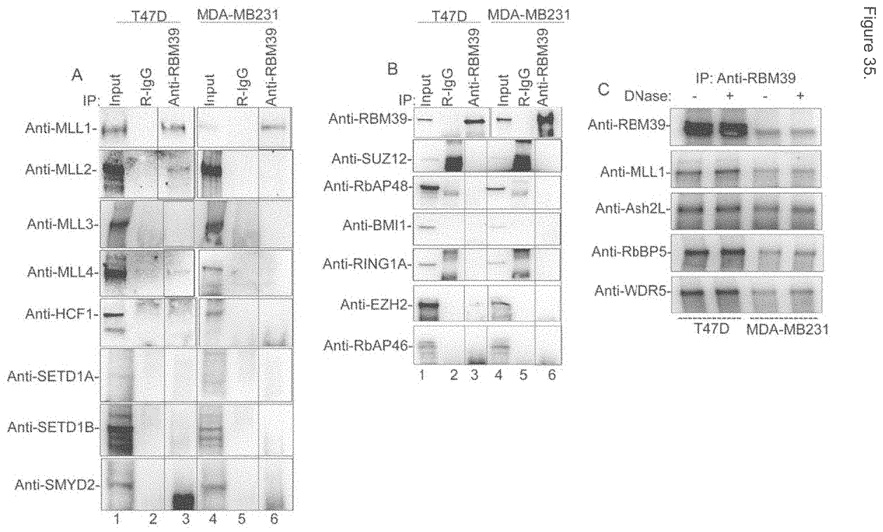

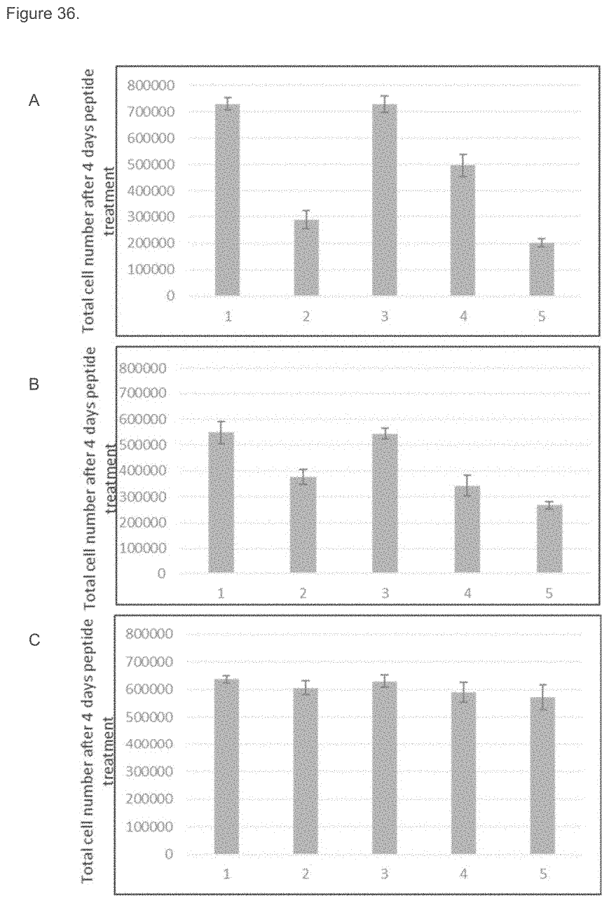

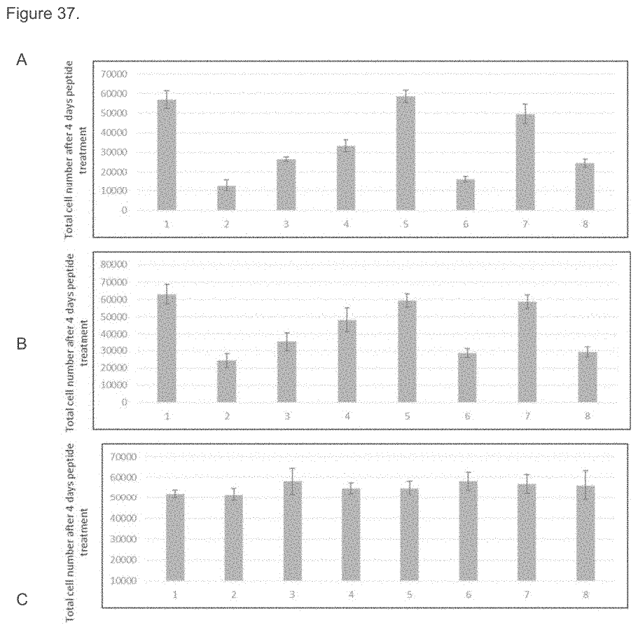

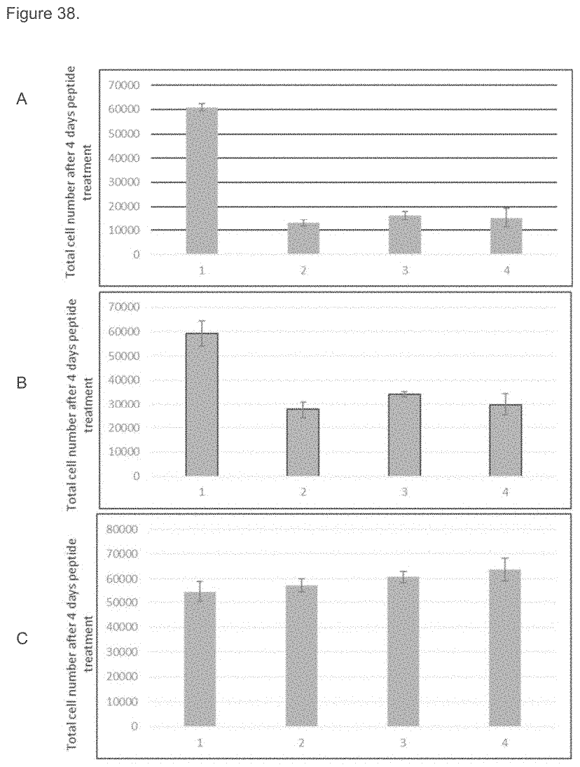

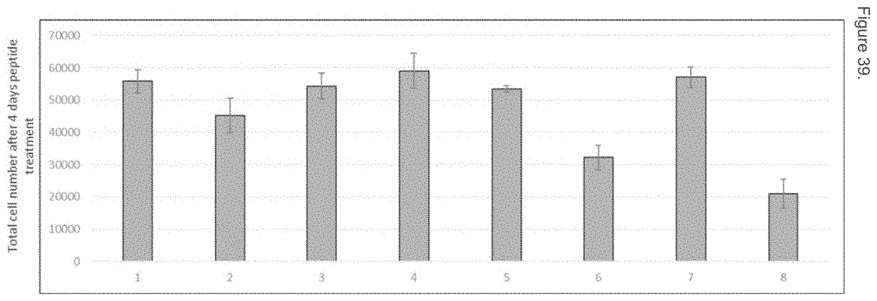

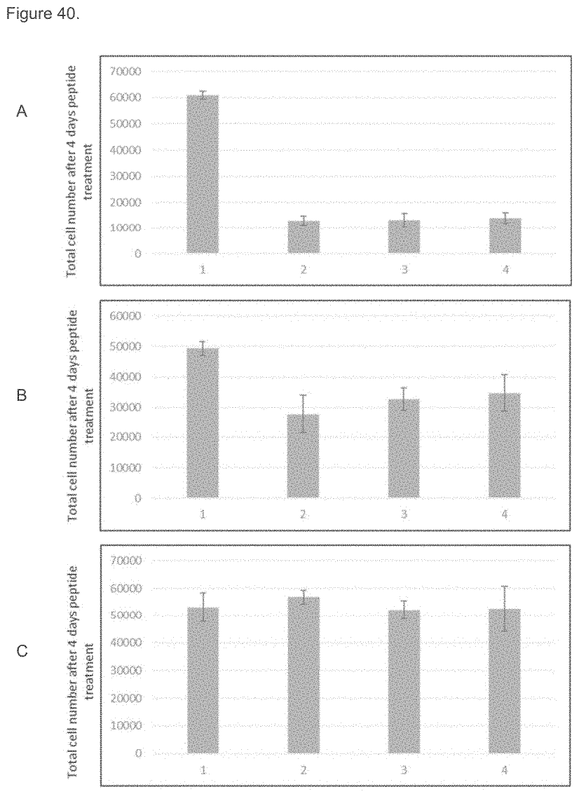

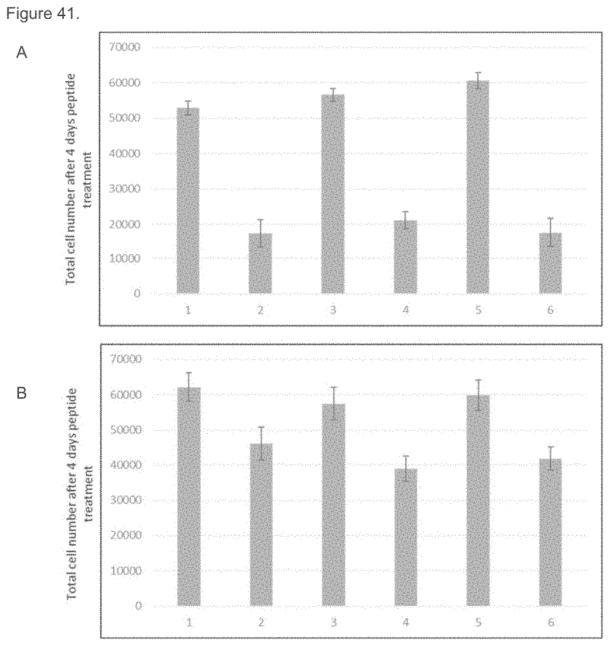

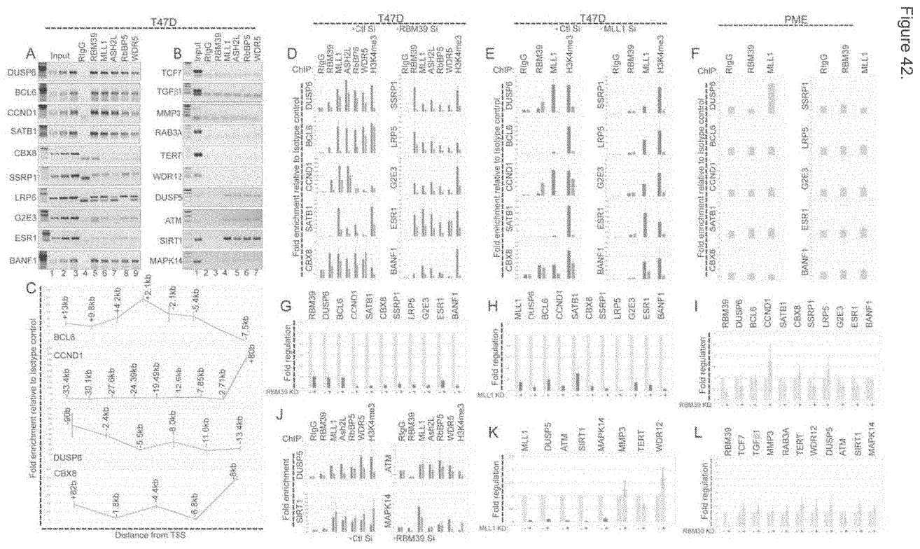

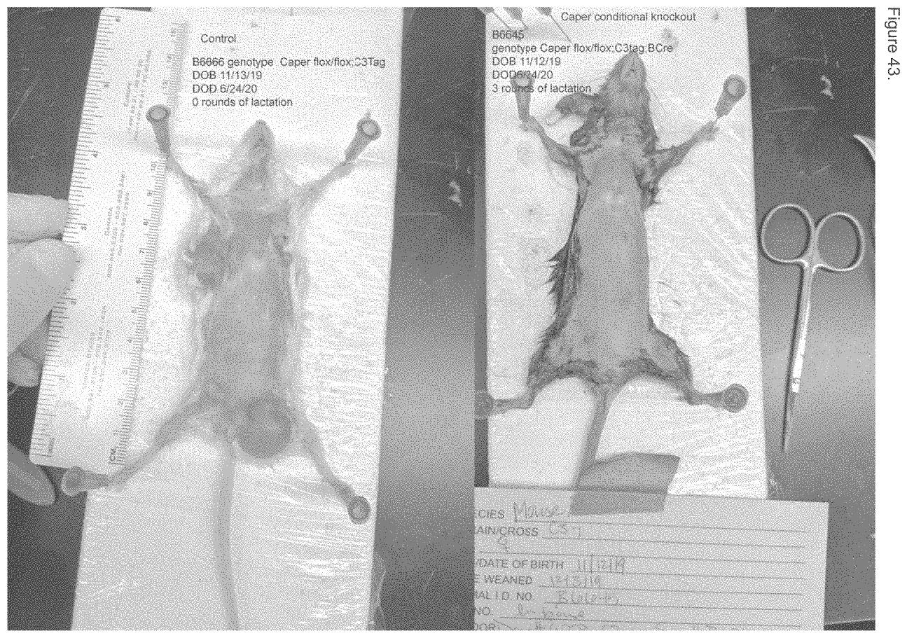

It was previously demonstrated that CAPERα kd in primary human fibroblasts resulted in altered H3K9 methylation and acetylation of promoter regions of target genes, repressing their transcription (Kumar et al., 2014). Whether decreasing CAPERα levels alters global histone epigenetic marks in breast cancer cells was investigated. The lysates from cells transfected with CAPERα or negative control siRNAs was analyzed. While the total amount of Histone-3 (H3) was unchanged, there was a dramatic decrease in the level of the activating H3K4me3 mark in both T47D and MDA-MB231 breast cancer lines (and in MDA-MB468, not shown) but not in PMEs (FIG. 11H). Histone association assays showed that CAPERα interacts with H3K4me3 and H3K9ace activating marks in breast cancer cells, but not PMEs (FIG. 11J). CAPERα kd decreased levels of H3K27ace and H3k9ace activating marks in T47Ds but had no effect on repressive marks (H3K9me3 or H3K27me3, FIG. 11H′). In contrast, in primary human fibroblasts (PHFs) the levels of activating marks increase, and repressive marks decrease after CAPERα kd (FIG. 34A) and CAPERα only interacts with repressive marks (FIG. 34C). This is consistent with the CAPERα/TBX3 corepressor previously reported in PHFs (Kumar et al., 2014).

### Example 4

### CAPERα Interacts with Histone Modifying Enzymes in a Cell-Specific Manner

The data presented herein indicate that CAPERα is involved in establishing or maintaining active epigenetic marks in breast cancer cells. Given the loss of H3K4me3 that was observed in CAPERα kd breast cancer cells, whether CAPERα interacts with MLL1 (i.e., whether MLL1 co-IPs with endogenous CAPERα (and the reciprocal) in T47 and MDA-MB231 breast cancer cells, but not in PMEs (FIG. 2A, B) was investigated. The CAPERα-MLL1 complex (henceforth called CAP/MLL1) was detected in breast cancer cell lines of all subtypes (Dai X, et al., J Cancer. 8(16):3131-41 (2017)), independent of p53 status or amount of CAPERα expressed (FIG. 12).

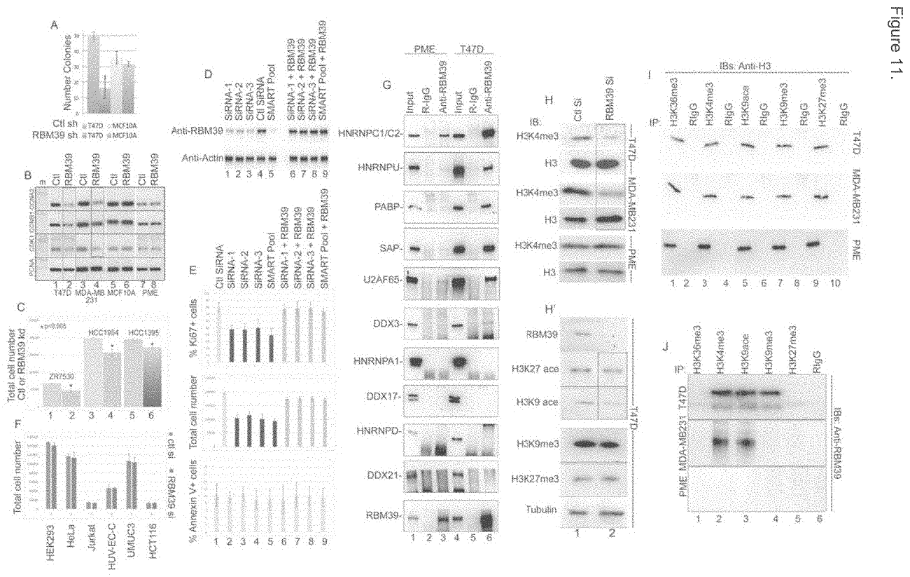

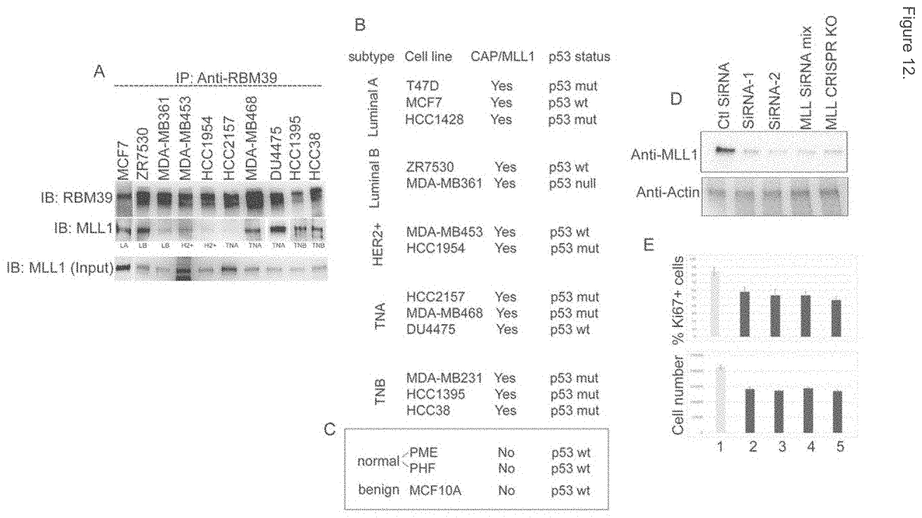

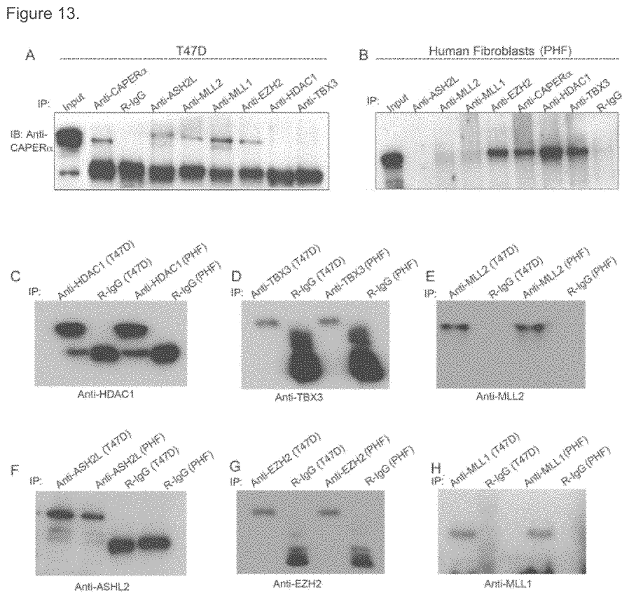

Importantly, co-IPs of lysates prepared from flash frozen human tissues showed no CAP/MLL1 interaction in normal breast, while CAPERα and MLL1 interacted in both ER+ and triple-negative breast cancers (FIG. 2C), indicating that the complex is not merely an epiphenomenon in breast cancer cell lines. CAP/MLL1 was undetectable in other human tissues, including those commonly adversely affected by chemotherapy (FIG. 2D). These results suggest that CAP/MLL1 complex may be specific to breast cancer cells. This is further supported by the observation that in addition to MLL1, CAPERα interacts with COMPASS factors ASH2L and MLL2 in T47D cells, whereas in primary fibroblasts, it interacts with corepressors HDAC-1 and TBX3 (FIGS. 13A, B). EZH2 can function as a coactivator or repressor in breast cancer cells (Tan J Z, et al., Acta pharmacologica Sinica. 35(2):161-74 (2014)) and interacts with CAPERα (FIG. 13A), while in primary fibroblasts it interacts with the CAPERα/TBX3 corepressor (FIG. 13B).

Whether MLL1 kd phenocopies CAPERα loss of function (FIG. 2E) was also tested. Akin to CAPERα, MLL1 kd decreased proliferation indices and H3K4me3 in breast cancer cells (FIG. 2FH). MCF10A cells were unaffected by MLL1 kd, while PMEs had a slight reduction in proliferation and H3K4me3 consistent with MLL1's known function in many normal cells. These data further support that the CAP/MLL1 complex plays a key role in regulating the status of histone marks and proliferation of breast cancer cells.

### Example 5

### CAPERα Interacts with Multiple Compass Complex Members in Breast Cancer Cells

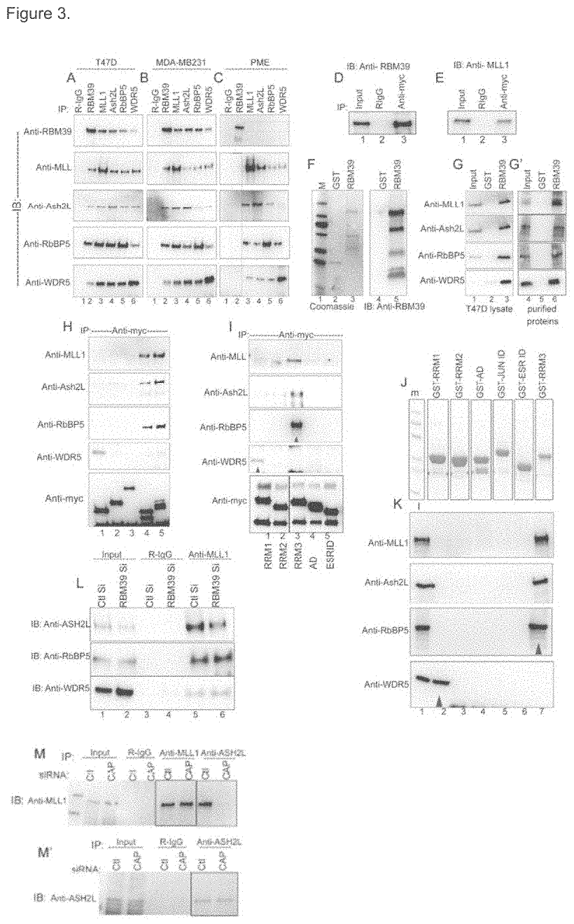

Optimal MLL1 catalytic activity requires that it function within a core complex containing COMPASS proteins ASH2L, RbBP5 and WDR5 (Dou Y, et al., Nat Struct Mol Biol. 13(8):713-9 (2006)). Endogenous CAPERα, MLL1, ASH2L, RbBP5 and WDR5 reciprocally co-IP each other in T47D (FIG. 3A) and MDA-MB231 breast cancer cells (FIG. 3B), but not in PMEs (FIG. 3C).

Myc- and GST-pull downs were used in HEK293 cells to assay for direct interactions. Overexpressed myc-tagged CAPERα co-IP′d endogenous MLL1 in heterologous cells (FIGS. 3 D, E) and all MLL1 core complex members present in T47D lysates bound GST-CAPERα (FIG. 3F) but not GST alone (FIG. 3G).

CAPERα has three RRM domains, a transcriptional activation (AD), and estrogen receptor (ESR-ID) and JUN (JUN-ID) interaction domains (Uniprot, FIG. 14). To identify domain(s) necessary for binding with MLL1 complex, myc-tagged CAPERα deletion and isolated domain expression constructs were generated and expressed in heterologous cells (FIGS. 14A, B). WDR5 interacts with an N-terminal portion of CAPERα (FIG. 3H, lane 1), while MLL1, ASH2L, and RbBP5 co-IP′d with C-terminal regions (FIG. 3H, lanes 4, 5). Consistent with this, WDR5 interacts with the isolated RRM1 domain (located in the N-terminus, FIG. 3I, lane 1), while MLL1, ASH2L and RbBP5 interacted with RRM3 (C-terminal, FIG. 3I, lane 3). Purified individual domain GST-fusion proteins (FIG. 14C) incubated with T47D lysates confirm the myc pulldown interactions and show that they are direct (FIG. 3J). These breast cancer specific protein-protein interactions are of great functional and potential therapeutic importance since WDR5 provides an RNA binding pocket for the core complex while the RbBP5/ASH2L heterodimer forms a “joint catalytic center” with the SET domain of MLL1 (Cao F, et al., PLoS One. 5(11):e14102 (2010); Li Y, et al., Nature. 530(7591):447-52 (2016); Wysocka J, et al., Cell. 121(6):859-72 (2005); Odho Z, et al., J Biol Chem. 285(43):32967-76 (2010)).

Whether CAPERα depletion influences interaction of MLL1 core components was investigated. CAPERα kd disrupted the MLL1-ASH2L interaction (FIG. 3L and FIG. 14D), but not MLL1-WDR5 or MLL1-RbBP5 (FIG. 3L). Thus, CAPERα not only interacts with MLL1 components, but promotes complex integrity by facilitating MLL1-ASH2L interaction.

Although CAPERα directly interacts with estrogen receptor in yeast (Jung D J, et al., J Biol Chem. 277(2):1229-34 (2002)) and MLL1 interacts with HCF-1 in leukemia cells (Yokoyama A, et al., Mol Cell Biol. 24(13):5639-49 (2004)), interaction between CAPERα and either of these factors in T47D, MDA-MB231 or PME cells was not detected (FIG. 14F).

### Example 6

### CAPERα is Required for MLL1 Occupancy and Regulation of Known MLL1 Target Genes

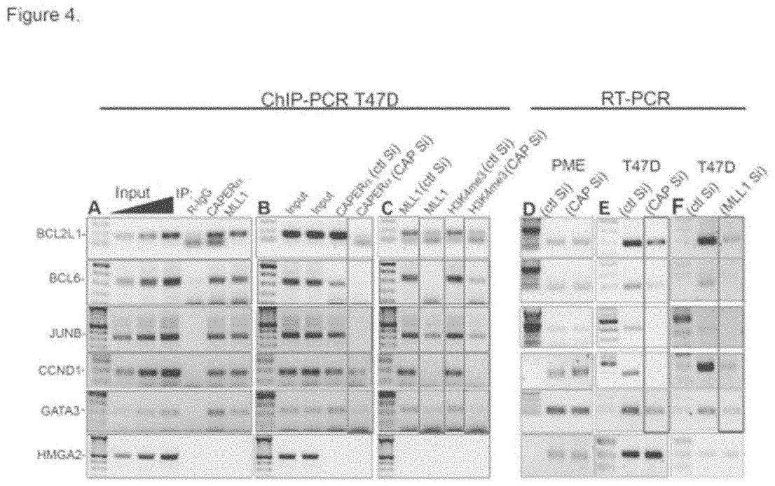

BCL2L1, BCL6, JUNB, CCND1 and GATA3 are known MLL1 direct targets in U937 cells (Guenther M G, et al., Genes Dev. 22(24):3403-8 (2008)). ChIP-PCR shows that CAPERα and MLL1 co-occupy the promoters of these genes in T47D cells (FIG. 4A). Furthermore, assay of control and CAPERα kd cells revealed that MLL1 binding and H3K4me3 of these targets not only coincide with CAPERα chromatin occupancy, they require it (FIGS. 4B, C). RT-PCR showed that expression of these genes is markedly decreased by kd of CAPERα or MLL1 in T47D but not PME cells (FIGS. 4D-F). Since CAPERα kd has no effect on MLL1 protein levels (FIG. 14A), its effects on H3K4me3 and gene expression in T47D cells result from failure of the MLL1 complex to bind in the absence of CAPERα.

### Example 7

### Genome-Wide Analysis of CAPERα and MLL1 Chromatin Occupancy in T47D Breast Cancer Cells

Decreased bulk H3K4me3 levels after CAPERα kd suggest that its requirement for MLL1 function is pervasive in breast cancer cells. High-throughput sequencing of CAPER and MLL1 bound chromatin IP′d from T47Ds identified 5,741 bound regions for CAPERα (associated with 7743 genes) and 11,037 for MLL1 (10,882 genes) (FIG. 5A, Tables 1-3). Remarkably, >4,500 regions were co-occupied by CAPERα and MLL1. This highly significant 79% co-occupancy substantiates the importance of the CAP/MLL1 complex in regulating chromatin structure and downstream transcriptional pathways controlling breast cancer cell phenotypes. Because most peaks were greater than 1 Kb wide (as has been reported for MLL1 occupancy in other cell types (Xu H, et al., Cancer Cell. 30(6):863-78 (2016)) (FIG. 5B), they were referred to as cobound “regions”. The majority of cobound regions (78%) were in promoters/5′UTRs (FIG. 5C). In 8% of all cobound genes, CAP/MLL1 occupies very broad windows encompassing the entire gene; this includes “pioneer” factor genes GATA3 (FIG. 5F′) and FOXA1 that function upstream of estrogen receptor in luminal-like breast cancers (Chen K, et al., Nat Genet. 47(10):1149-57 (2015); Lever J, et al., Nat Methods. 16(6):505-7 (2019); Heinz S, et al., Mol Cell. 38(4):576-89 (2010); McLean C Y, et al., Nat Biotechnol. 28(5):495-501 (2010); Sanchez-Vega F, et al., Cell. 173(2):321-37 e10 (2018)). This broad occupancy is similar to H3K4me3/H3K27ace patterns that correlate with high levels of expression, maintenance of transcription, and transcriptional elongation in many normal cell types (Chen et al., 2015).

Of the 137 cancer driver genes enumerated by Vogelstein (Vogelstein B, et al., Science. 339(6127):1546-58 (2013)), 47% are co-occupied by CAP/MLL1 (p<2.0×10-09). The number of housekeeping, tumor suppressors and oncogenes (Lever J, et al., Nat Methods. 16(6):505-7 (2019)) bound by CAP/MLL11 is significantly higher than random (p values of 2.6×10-110, 6.12×10-24 and 2.43×10-60, respectively).

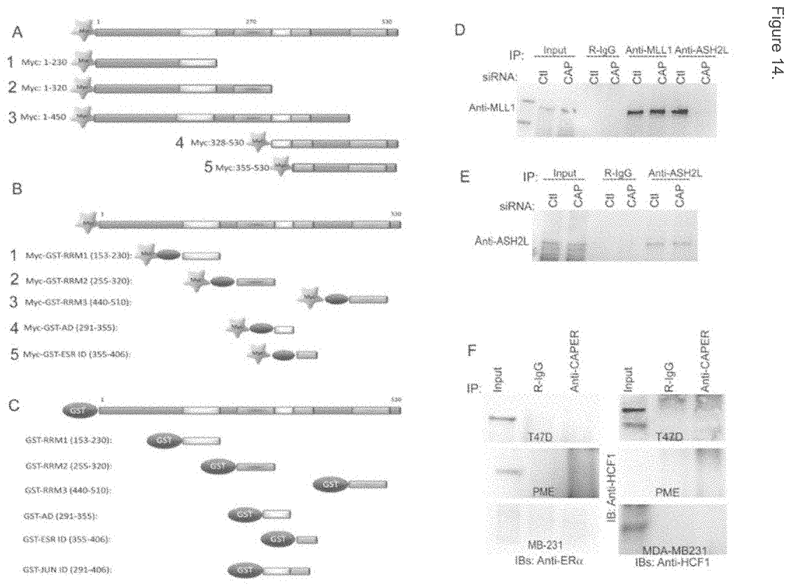

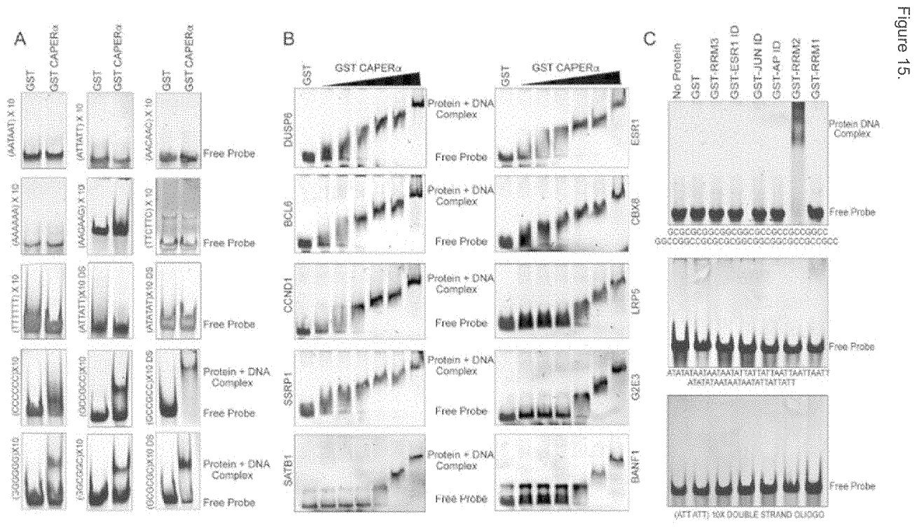

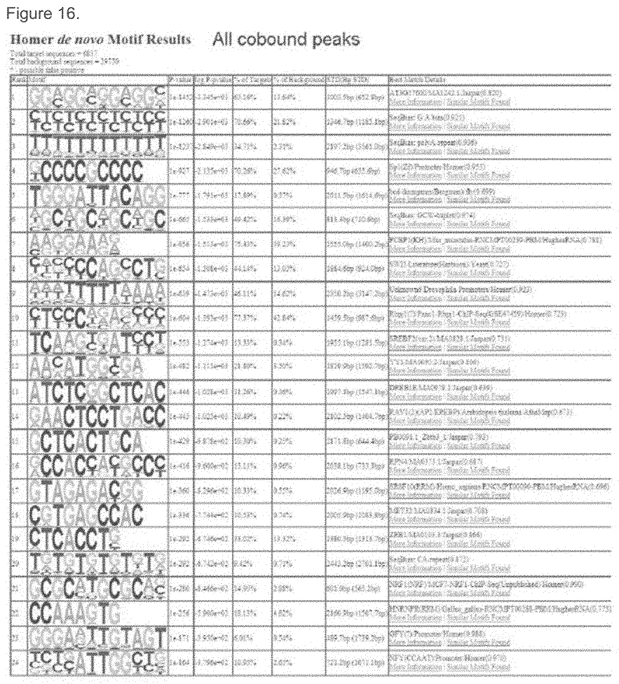

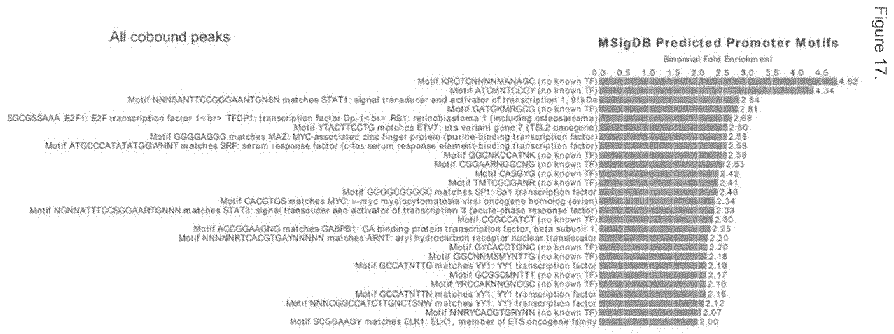

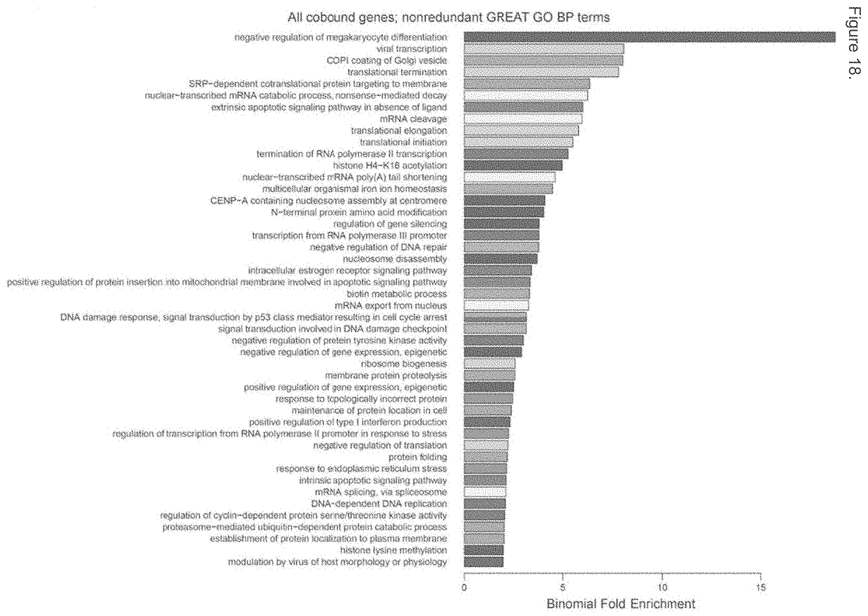

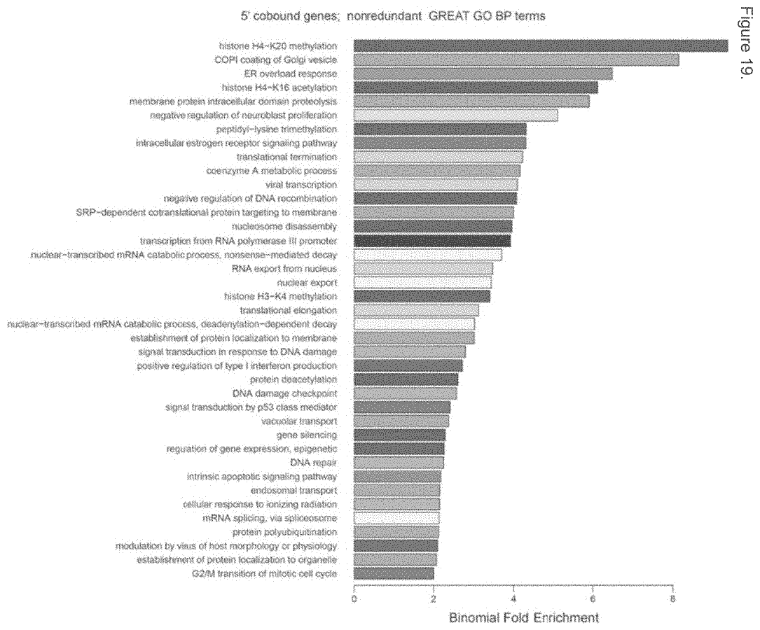

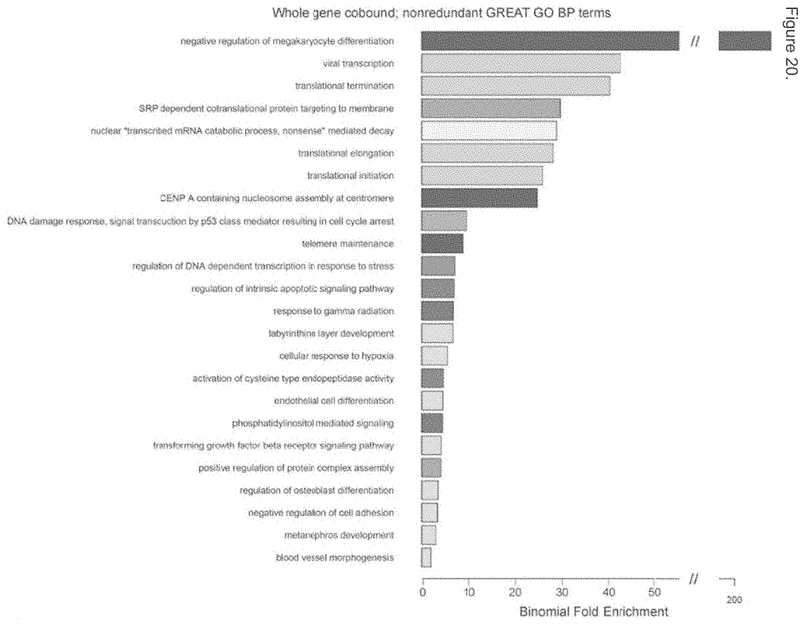

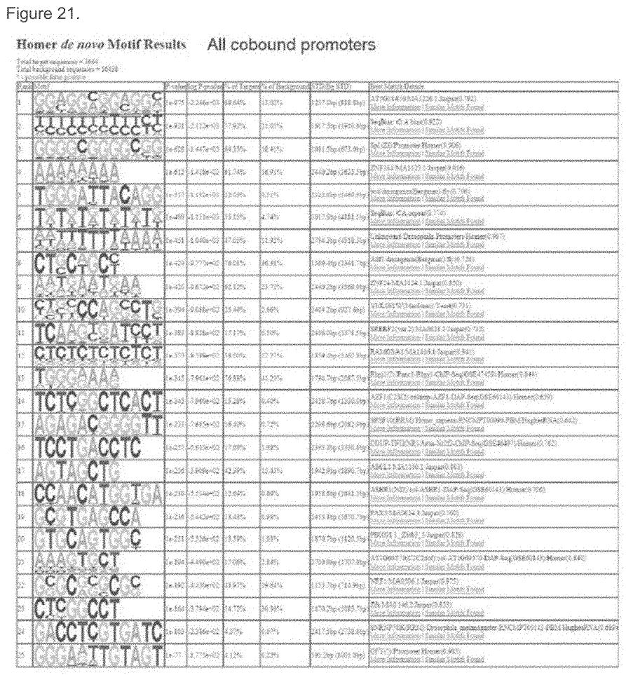

All co-bound regions and promoters for overrepresented motifs to were searched to identify sequences that may facilitate CAP/MLL1 binding. The most enriched de novo motif identified by HOMER (Heinz S, et al., Mol Cell. 38(4):576-89 (2010)) (FIG. 5D) is consistent with affinities revealed by EMSA assays showing recombinant CAPERα binds GC-rich oligonucleotides (FIG. 15A). The complete HOMER motif output for all cobound regions is shown in FIG. 16; GREAT analysis (McLean et al., 2010) yielded additional conserved motifs (FIG. 17). Enriched cobound promoter motifs (FIG. 21 and FIG. 22) include consensus motifs for MYC, Rb, E2F1 predicting direct targets of CAP/MLL1 in cell cycle regulation, while STAT1/3, ARNT, GABPB1/2 motifs predict cytokine, hypoxia and mitochondrial/metabolic targets, respectively.

The GREAT hierarchical tool allowed the identification of the most specific significantly enriched Gene Ontology (GO) Biologic Process (BP) terms. Then, the members of each term's geneset were manually examined and curated into functional categories (color key, FIG. 5D″). The pie charts in FIGS. 5 D-F show the number of BP terms in each category. The enriched BP terms and how they were coded by function/color are shown in S. FIGS. 5.4-5.6. Representative occupancy patterns for 5′ (promoter/TSS) cobound and whole gene cobound are shown in FIGS. 5 E′ and F′.

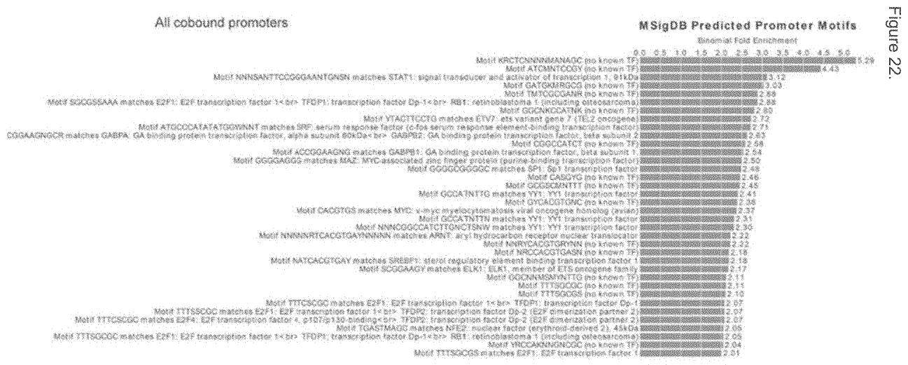

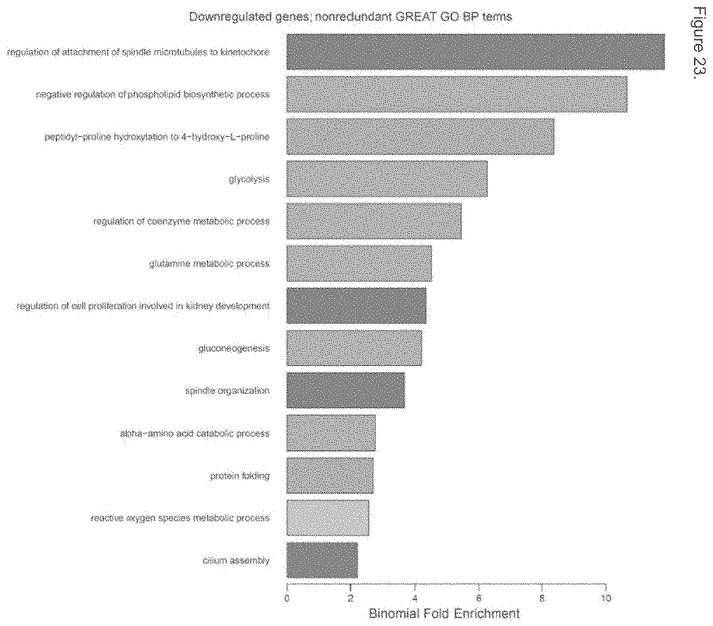

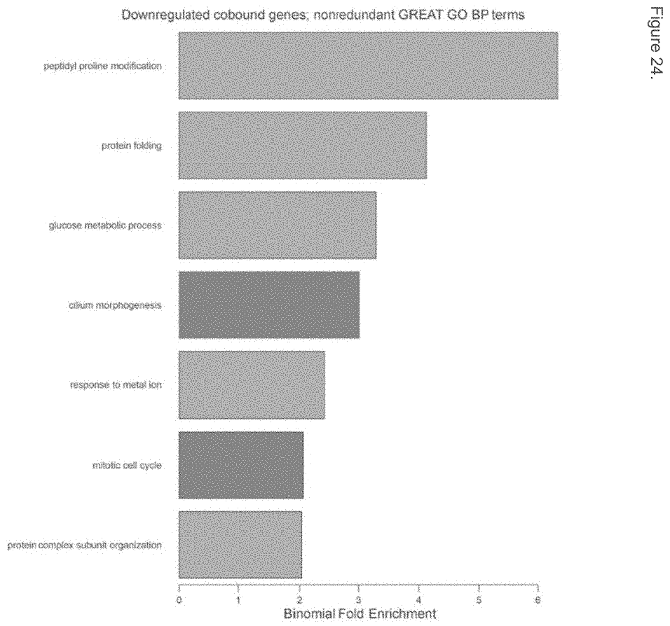

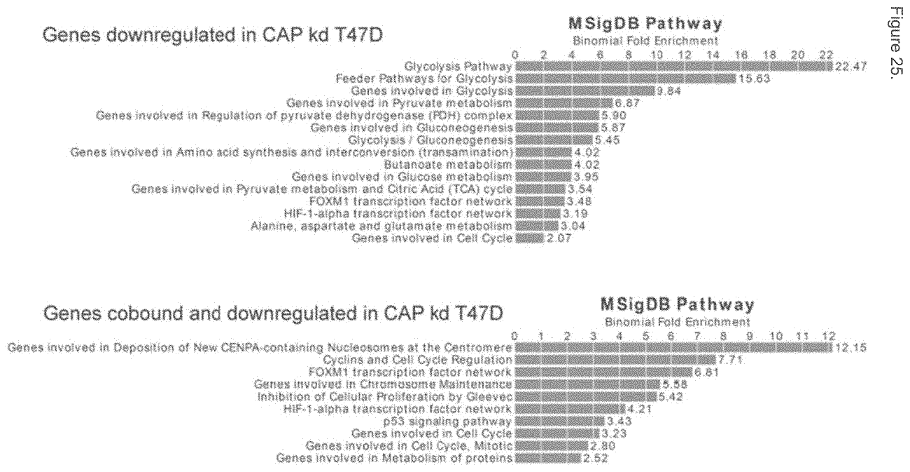

To investigate the functional relevance of CAP/MLL1 occupancy, genome-wide transcriptional profiling of T47D cells treated with control or CAPERα siRNAs was performed. 3,873 genes were differentially expressed at a significance of q<0.05 (FIG. 5G, Tables 1-3); this includes ˜30% of cancer “driver” genes (Vogelstein et al., 2013), correlating well with the ChIP-Seq findings. Among cobound, differentially expressed genes, 748 (40%) were decreased and 728 (37%) increased (FIG. 5H, Tables 1-3); this level of correlation between factor occupancy and altered expression is highly significant (p values of 1.25×10-54 and 1.73×10-40, respectively). GO analysis showed that cell cycle, metabolic, hypoxia and protein trafficking/modification genes were enriched in the downregulated and cobound/downregulated genesets (FIG. 5 I, J; FIGS. 23 and 24). Consistent with CAP/MLL1 binding and regulating expression of pioneer/master factors GATA3, FOXA1, FOXM1, ESR1 and MYC (FIGS. 4, 5D-F′,6, Tables 1-3), major pathways driving breast cancer hallmarks were decreased (FIG. 25) as were 23 cancer neighborhoods (FIG. 5I′). In contrast, loss of CAPERα increased expression of NFKβ, TNF, interferon and other cytokine/immunomodulatory BPs as well as multiple tumor suppressors in the Wnt and Notch pathways (FIGS. 5 K, L; FIGS. 26-28, Tables 1-3). These classes of direct targets are consistent with those predicted by the enriched promoter motifs identified by ChIPSeq.

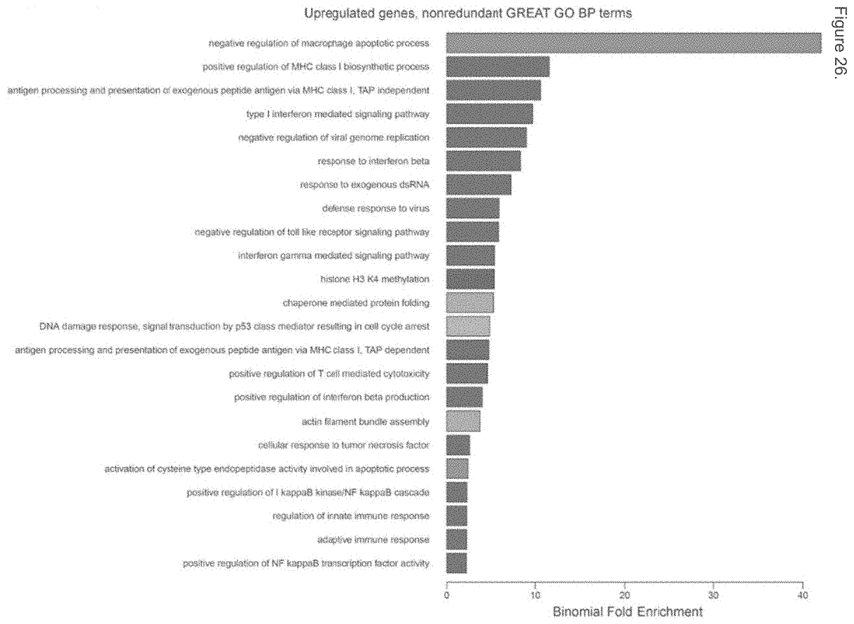

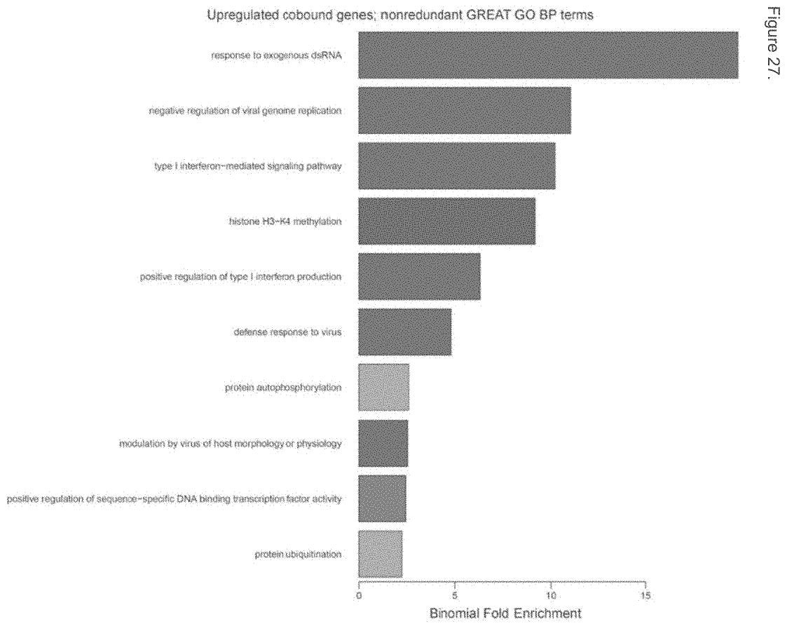

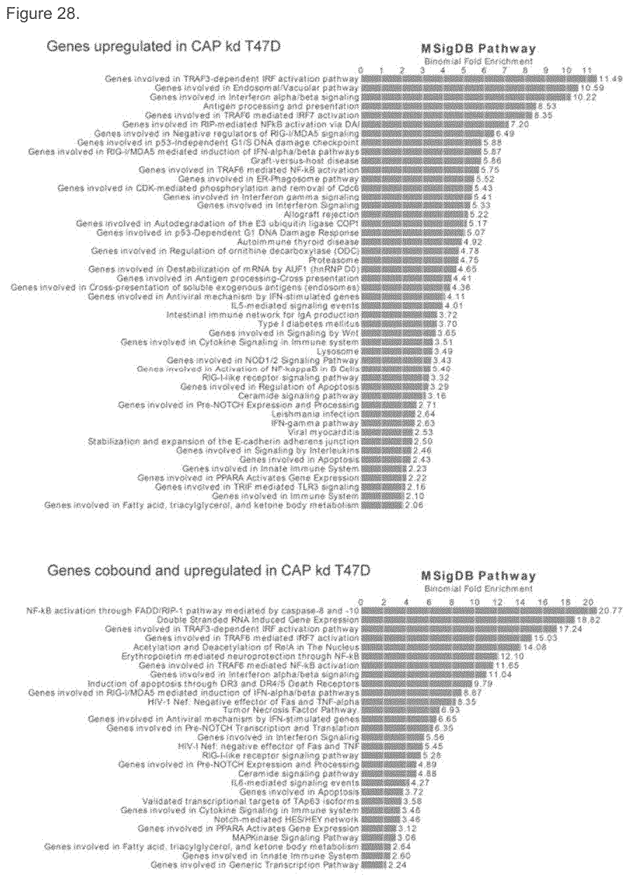

Notably CASPASE genes 2, 3, 4, 6, 8 and 9 were cobound/upregulated and enrichment of proapoptotic genes and pathways was detected by GREAT (FIG. 5, FIG. 28, Tables 1-3). KMT family members KMT2B, C and D and SETD1A and 1B were cobound/upregulated, suggesting a potential compensatory mechanism to preserve H3K4 methylation and pathologic complex function in breast cancer cells; these genes largely accounted for the “histone H3-K4 methylation” enriched term in these genesets (FIGS. 26-27, Tables 1-3).

From a broader view, of the 10 canonical oncogenic signaling pathways curated by TCGA (Sanchez-Vega F, et al., Cell. 173(2):321-37 e10 (2018)), 3 had cobound tumor suppressors genes whose expression increased with CAPERα kd (Wnt, Notch and Hippo pathways) while the MYC, p53 and Cell cycle pathways had multiple cobound/downregulated oncogenes. Thus, loss of CAPERα reverts the molecular signature of breast cancer cells by changing expression of high-level, direct targets.

### Example 8

### The CAPERα/MLL1 Complex Coregulates Transcription of Novel Targets

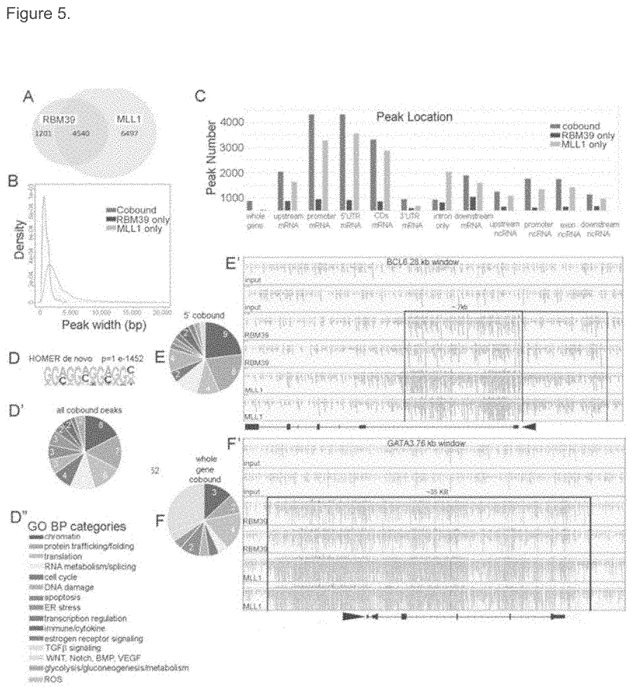

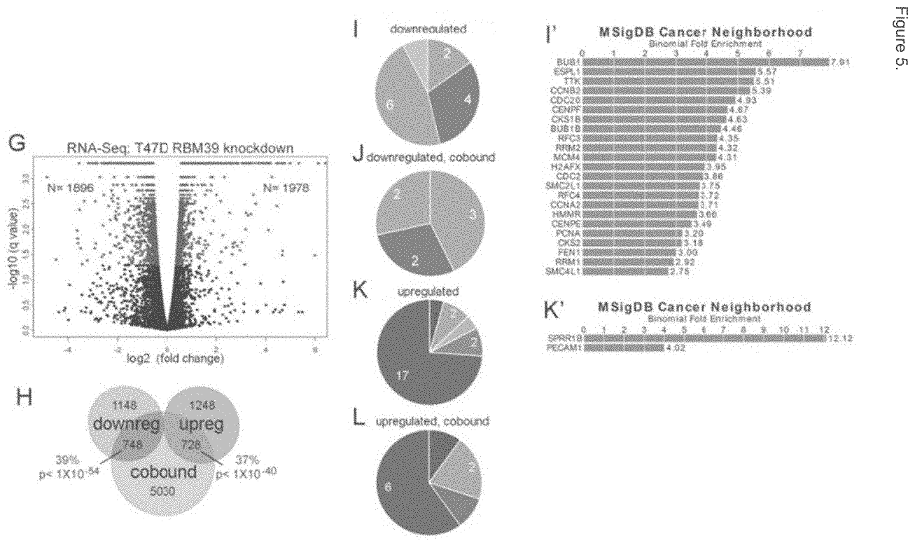

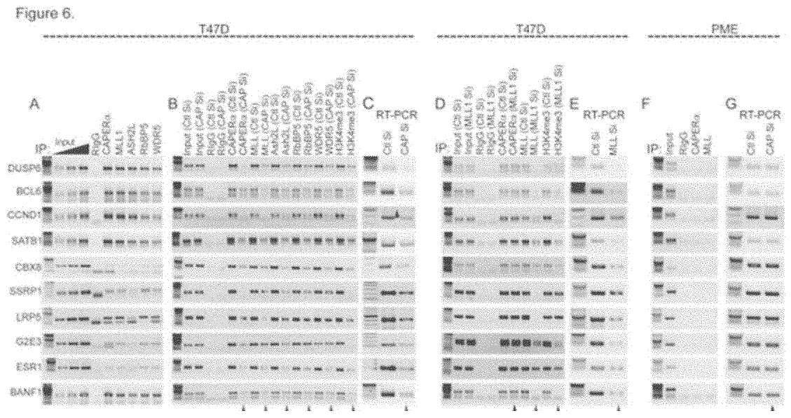

To further validate the importance of the CAP/MLL1 complex in breast cancer cell epigenetics and transcriptional regulation, a sample of CAP/MLL1 target promoters (DUSP6, BCL6, CCND1, SATB1, CBX8, SSRP1, LRP5, G2E3, ESR1, BANF1) were selected and ChIP-PCR was used to assay chromatin occupancy by MLL1 core complex members: all members bound these promoters in T47D but not PME cells (FIG. 6A versus F). CAPERα kd markedly decreased association of MLL1, ASH2L, RbBP5, WDR5 with these promoters with a concomitant decrease in H3K4me3 (FIG. 6B) and transcript levels (FIG. 6C). It had no effect in PMEs (FIG. 6G). MLL1 kd did not disrupt CAPERα occupancy (FIG. 6D), but it had the expected decremental effect on H3K4me3 and transcript levels in T47D cells (FIGS. 6D, E). Thus, MLL1 complex binding, H3K4me3 catalysis, and transcriptional activation of these CAP/MLL1 targets depend on CAPERα chromatin occupancy.

### Example 9

### CAPERα Directly Binds to DNA Via its RRM2 Domain

CAPERα was further investigated for its intrinsic DNA binding function. EMSAs with DNA oligos and purified GST-CAPERα protein revealed that CAPERα strongly binds GC rich single and double stranded probes, but not A/T or all C containing probes (FIG. 15A). Testing for direct physical association between GST-CAPERα and PCR-amplified regions of cobound targets of CAP/MLL1 showed direct binding to these promoters (FIG. 15B), a finding consistent with CAPERα functioning as a bona fide DNA-binding transcription factor that recruits MLL1 complex to target promoters to regulate epigenetic marks and transcription. Of the previously described purified GST-CAPERα domains, only RRM2 bound DNA (FIG. 15C).

### Example 10

### CAPERα and MLL1 Complex Members are Overexpressed and Mislocalized in Breast Cancer Cells

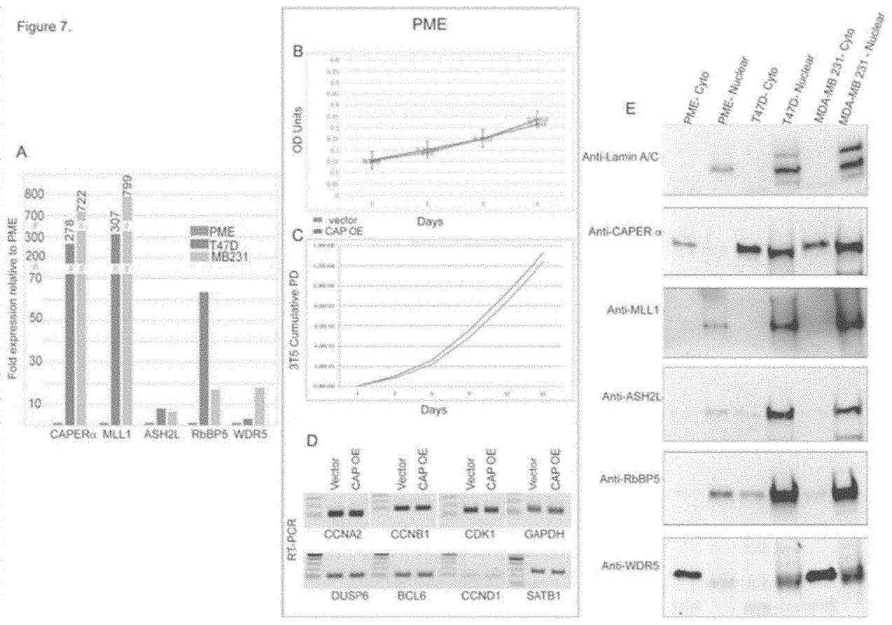

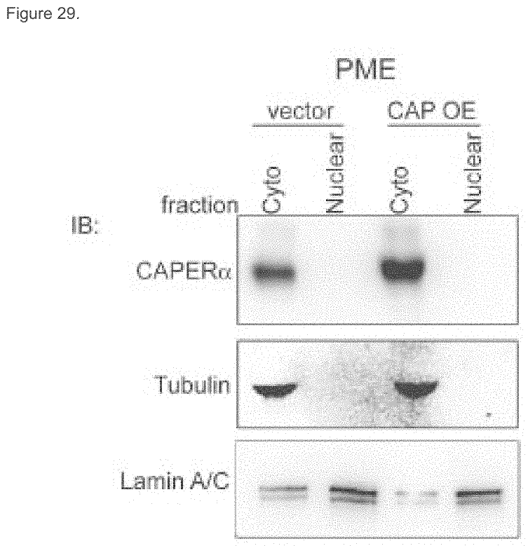

A key question is what drives formation of the CAP/MLL1 complex in breast cancer and not normal breast cells. To begin to address this, the expression and subcellular localization of CAP/MLL1 complex members in PMEs and breast cancer cells was examined. RT-PCR demonstrates that transcript levels for complex members are markedly higher in breast cancer cells than PMEs (FIG. 7A). However, overexpression of CAPERα protein was not sufficient to increase proliferation or alter gene expression in PMEs (FIGS. 7B-D, FIG. 29). Cytoplasmic and nuclear fractions for CAPERα and MLL1 components in T47D, MDA-MB231 and PME cells was assayed to determine if subcellular localization plays a role (FIG. 7E). CAPERα was cytoplasmic in PMEs but, in addition to a dramatic increase in the amount of protein (consistent with the RNA data), it was both cytoplasmic and nuclear in T47D and MDA-MB231 breast cancer cells. Differential WDR5 localization mirrors that of CAPERα. MLL1 is nuclear in all 3 cell types and its levels were increased in breast cancer cells, as were ASH2L and RbBP5. Combined with the finding that overexpressed CAPERα does not traffic to the nucleus in PMEs (FIG. 29), these data indicate that CAPERα acquires new functions in the nucleus of breast cancer cells where it has access to MLL1 complex members.

### Example 11

### Direct Interaction of CAPERα and MLL1 is Necessary for Gene Activation and Breast Cancer Cell Proliferation

Since RRM3 mediates the CAP/MLL1 interaction (FIGS. 3H-K), it was investigated whether this domain, in isolation, could have a dominant-negative activity. If so, it would provide further evidence of the importance of a direct association between CAPERα and MLL1 and conclusively establish that it is the CAP/MLL1 complex that is critical for breast cancer cell proliferation and gene regulation. It would also offer a novel therapeutic strategy to disrupt this complex in breast cancer cells.

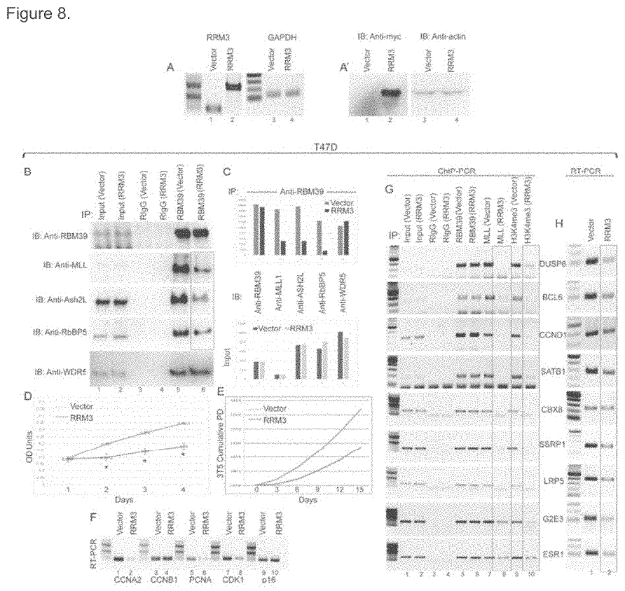

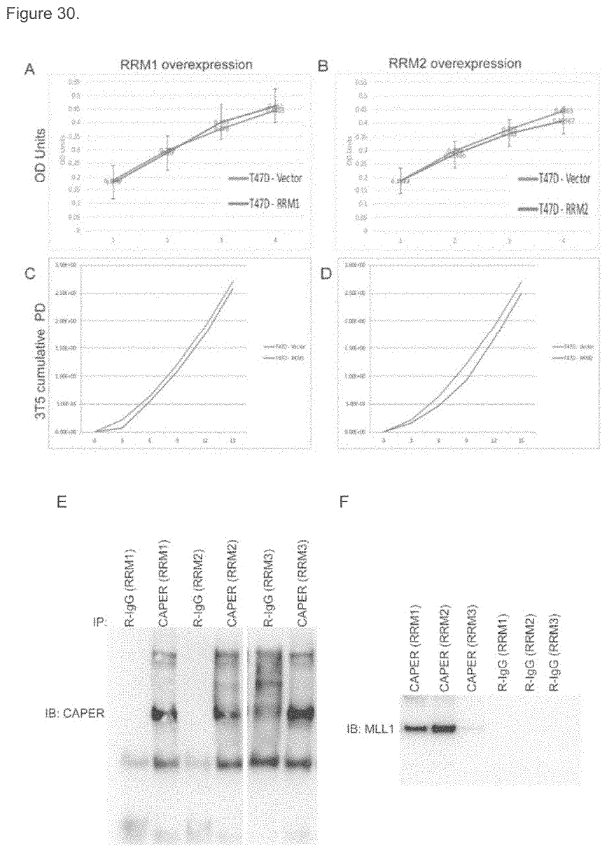

Toward these objectives, empty or RRM3-expressing vectors (FIGS. 8A, A′) were transfected into T47D cells. Expression of RRM3 did not affect endogenous levels of CAPERα, MLL1, ASH2L, RbBP5 or WDR5 (FIG. 8B, input lanes) but it disrupted interactions between CAPERα and MLL1 complex members with the exception of WDR5 (FIGS. 8B, C); this is congruent with RRM1 mediating the CAPERα-WDR5 interaction and the MLL1-WDR5 interaction being CAPERα independent (FIGS. 3H-K). As expected with it disrupting CAP/MLL1, RRM3 decreased proliferation of breast cancer cells (FIGS. 8D, E) and expression of pro-cell cycle genes (FIG. 8F). RRM3 had no effect on CAPERα occupancy of target promoters (as expected, since CAPERα binds independent of MLL1), but RRM3-mediated complex disruption prevented MLL1 binding, H3K4me3 (FIG. 8G), and expression of target genes (FIG. 8H). RRM3 also disrupts growth and gene expression in MDA-MB231 breast cancer cells, but not in PMEs (FIGS. 8I-N). Overexpression of RRM1 or RRM2 had none of these effects (FIG. 30).

Overall, the data indicate that CAPERα binds DNA and MLL1 complex members, to recruit the complex to target promoters. Further, these results clearly show that it is the CAP/MLL1 complex that is pivotal for the establishing H3K4me3 marks, activating target gene transcription and stimulating abnormal proliferation of cancer cells.

### Example 12

### RRM3 Cell Penetrating Peptide Disrupts Breast Cancer Cell Growth

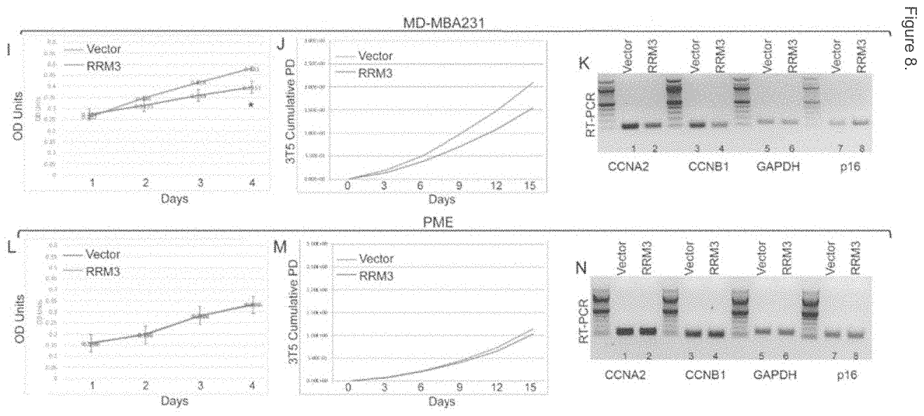

Whether recombinant RRM3 conjugated with the Penetratin cell penetrating peptide (Dupont E, et al., Methods Mol Biol. 1324:29-37 (2015)) (FIGS. 9A, B) affects growth of T47D and PME cells was tested. Equal numbers of PME and T47D cells were seeded and incubated with 5 μM peptide in media for 72 hrs. Qualitative and quantitative analyses showed CPP-RRM3 dramatically decreased the number of breast cancer cells yet had no effect on the number of PMEs (FIGS. 9C-G). Compared to CPP-coupled RRM3, CPP-RRM1 and RRM3 had no effect (FIGS. 9H-N).

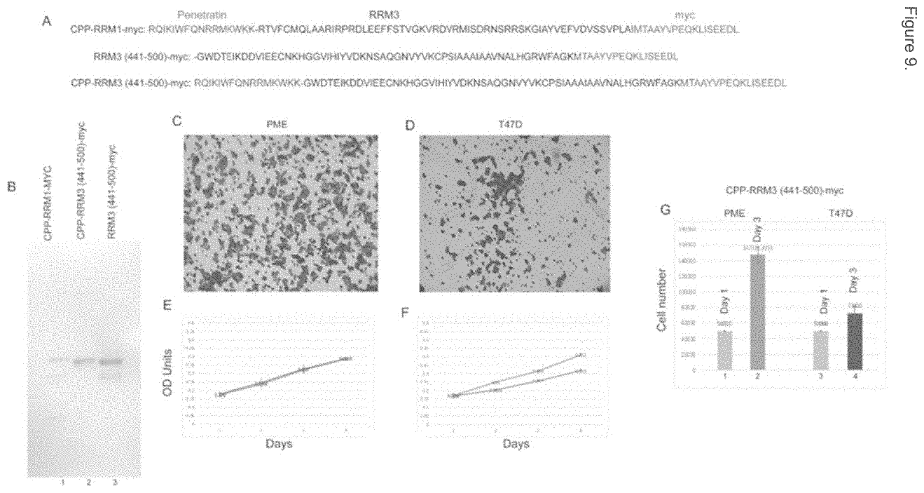

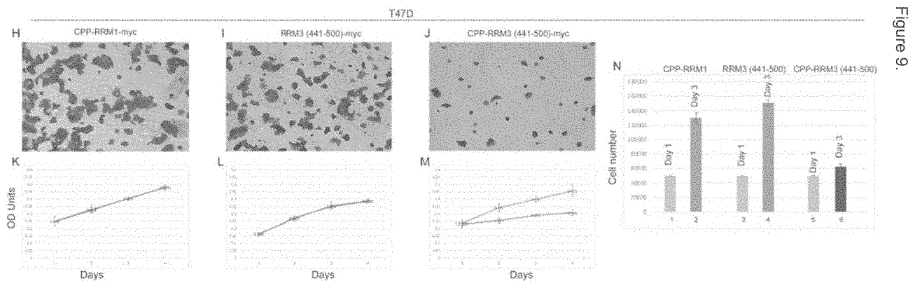

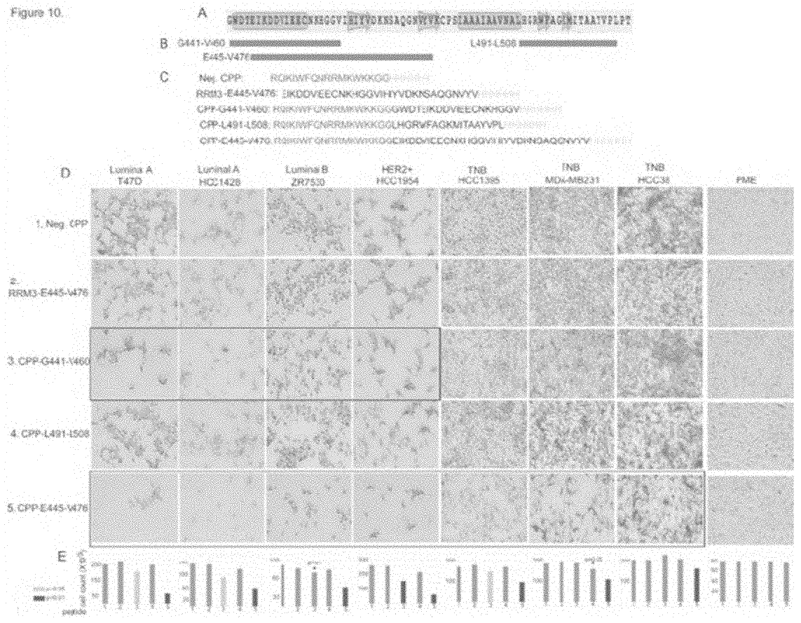

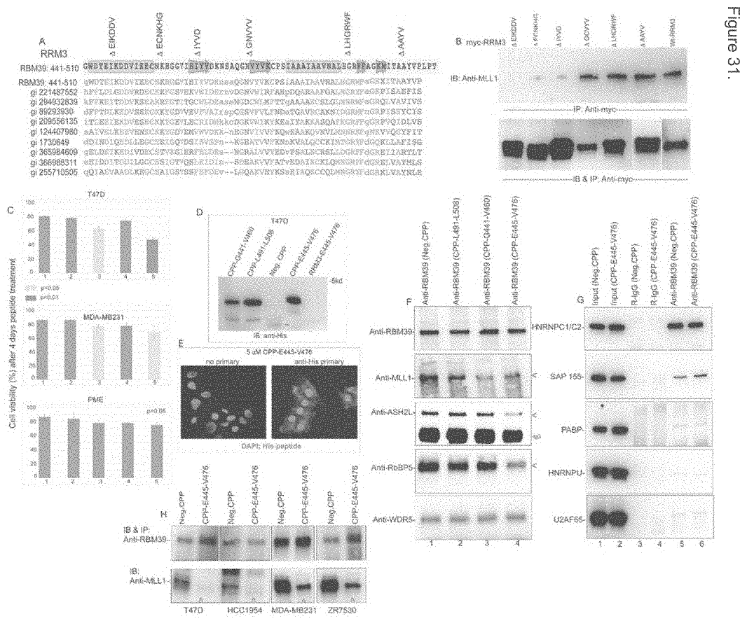

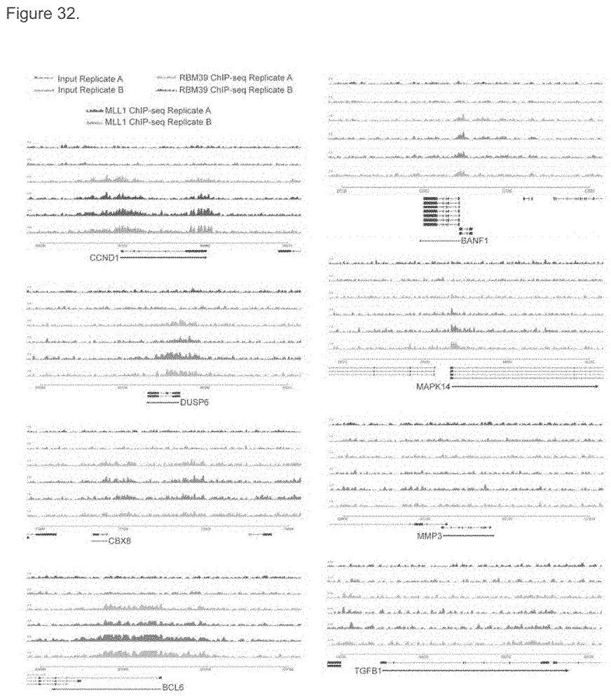

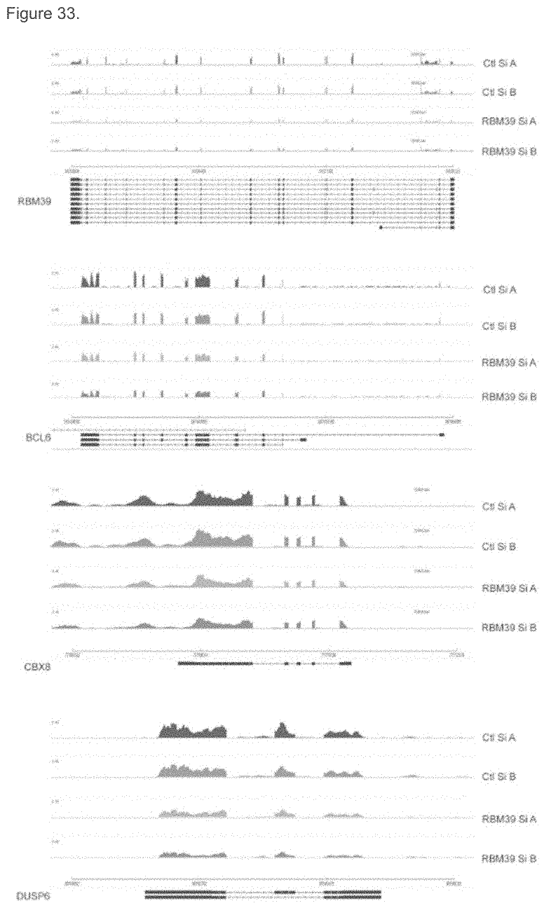

These results led to optimization of RRM3-derived peptides. As per UniProt, RRM3 spans amino acids (aa) 445-508 (SEQ ID NO: 34) and SWISS tool predicts 2 α helices and 4 β sheets that are conserved in vertebrate RRM-containing proteins (FIG. 31A). A series of myc-tag microdeleted constructs were designed to identify regions required for MLL1 binding. Deletions affecting the 5′ sheet or helix markedly decreased interaction with MLL1 (FIG. 31B) while 3′ regions were not required, allowing the synthesis of still smaller RRM3-derived peptides with Penetratin and His tags: G441-V460; E445-V476 and L491-L508 and negative controls, Penetratin-His and RRM3 E445-V476-His (FIGS. 10A-C). An array of breast cancer cell subtypes were treated as described above. CPP-E445-V476 had pronounced effects on all subtypes, both in terms of proliferation (FIGS. 10D.5, E) and viability (FIG. 31C), but no effect on PMEs. CPP-G441-V460 affected some Luminal and HER2+ subtypes (FIGS. 10D.3, E). In the absence of Penetratin, RRM3 E445-V476 (SEQ ID NO: 2) had no effect (FIG. 10D.2) consistent with it failing to enter the nucleus (FIG. 31D). The ability of RRM3-derived peptides to decrease proliferation and viability only in breast cancer cells is expected since PMEs do not have CAP/MLL1, and it requires Penetratin for nuclear entry and access to the CAP/MLL1 complex.

LENGTHY TABLES
The patent application contains a lengthy table section. A copy of the table is available in electronic form from the USPTO web site (https://seqdata.uspto.gov/?pageRequest=docDetail&DocID=US20220265769A1). An electronic copy of the table will also be available from the USPTO upon request and payment of the fee set forth in 37 CFR 1.19(b)(3).

What is claimed is:

1. A method of treating cancer comprising administering a therapeutically effective amount of an RRM3-derived peptide of Coactivator of AP1 and Estrogen Receptor (CAPERα) to a subject in need thereof.

2. The method of claim 1, wherein the cancer is breast cancer.

3. The method of claim 2, wherein the breast cancer is estrogen receptor positive (ER+).

4. The method of claim 2, wherein the breast cancer is human epidermal growth factor receptor 2 positive (HER+).

5. The method of claim 2, wherein the breast cancer is triple-negative breast cancer (TNBC).

6. The method of any of claims 1-5, wherein administration of the RRM3-derived peptide of CAPERα disrupts the CAPERα/mixed lineage leukemia 1 (MLL1) (CAP/MLL1) complex.

7. The method of any of claims 1-6, wherein administration of the RRM3-derived peptide of CAPERα regulates chromatin marks.

8. The method of any of claims 1-7, wherein administration of the RRM3-derived peptide of CAPERα regulates transcription one or more of the following genes: BCL2L1, BCL6, JUNB, CCND1, FOXA1, FOXM1, ESR1, MYC, and GATA3.

9. The method of any of claims 1-7, wherein administration of the RRM3-derived peptide of CAPERα regulates transcription of one or more of the genes listed in Table 1.

10. The method of any of claims 1-7, wherein administration of the RRM3-derived peptide of CAPERα regulates transcription of one or more of the genes listed in Table 2.

11. The method of any of claims 1-7, wherein administration of the RRM3-derived peptide of CAPERα regulates transcription of breast cancer pioneer genes.

12. The method of any of claims 1-11, wherein administration of the RRM3-derived peptide of CAPERα prevents MLL1 occupancy in the CAP/MLL1 complex.

13. The method of any of claims 1-12, wherein administration of the RRM3-derived peptide of CAPERα inhibits the catalytic activity of the CAP/MLL1 complex.

14. The method of any of claims 1-13, wherein administration of the RRM3-derived peptide of CAPERα inhibits CAP/MLL1 complex binding to chromatin bearing one or more of the following MYC, Rb, E2F1, STAT1/3, ARNT, and GABPB1/2.

15. The method of any of claims 1-14, wherein administration of the RRM3-derived peptide of CAPERα disrupts binding to the CAP/MLL1 complex by ASH2L.

16. The method of any of claims 1-15, wherein administration of the RRM3-derived peptide of CAPERα decreases association of MLL1, ASH2L, RbBP5, and/or WDR5 with genomic promoters.

17. The method of any of claims 1-16, wherein administration of the RRM3-derived peptide of CAPERα inhibits histone 3, lysine 4 (H3K4) trimethylation of target genes.

18. The method of any of claims 1-17, wherein administration of the RRM3-derived peptide of CAPERα inhibits histone 3, lysine 9 (H3K9) acetylation of target genes.

19. The method of any of claims 1-18, wherein administration of the RRM3-derived peptide of CAPERα increases expression of one or more of the following proteins: NFKβ, TNF, and interferon.

20. The method of any of claims 1-19, wherein administration of the RRM3-derived peptide of CAPERα upregulates the transcription of one or more of the following genes: CASPASE gene 2, CASPASE gene 3, CASPASE gene 4, CASPASE gene 6, CASPASE gene 8, CASPASE gene 9, KMT2B, KMT2C, KMT2D, SETD1A, vREL, and SETD1B.

21. The method of any of claims 1-20, wherein the RRM3-derived peptide of CAPERα is nontoxic to noncancerous cells.

22. The method of any of claims 1-21, wherein administration of the RRM3-derived peptide of CAPERα results in decreases proliferation of cancer cells by about 30%.

23. The method of any of claims 1-22, wherein administration of the RRM3-derived peptide of CAPERα results in increases cell death by about 20%.

24. The method of any of claims 1-23, wherein the RRM3-derived peptide of CAPERα does not include the 5′ β-sheet of the RRM3 domain of CAPERα.

25. The method of any of claims 1-24, wherein the RRM3-derived peptide of CAPERα does not include the 5′ α-helix of the RRM3 domain of CAPERα.

26. The method of any of claims 1-23, wherein the RRM3-derived peptide of CAPERα comprises the amino acid sequence of SEQ ID NO: 1.

27. The method of any of claims 1-23, wherein the RRM3-derived peptide of CAPERα comprises the amino acid sequence of SEQ ID NO: 2.

28. The method of any of claims 1-23, wherein the RRM3-derived peptide of CAPERα comprises the amino acid sequence of SEQ ID NO: 3.

29. The method of any of claims 1-23, wherein the RRM3-derived peptide of CAPERα comprises the amino acid sequence of SEQ ID NO: 4.

30. The method of any of claims 1-23, wherein the RRM3-derived peptide of CAPERα comprises the amino acid sequence of SEQ ID NO: 5.

31. The method of any of claims 1-23, wherein the RRM3-derived peptide of CAPERα comprises the amino acid sequence of SEQ ID NO: 6.

32. The method of any of claims 1-23, wherein the RRM3-derived peptide of CAPERα comprises the amino acid sequence of SEQ ID NO: 7.

33. The method of any of claims 1-23, wherein the RRM3-derived peptide of CAPERα comprises the amino acid sequence of SEQ ID NO: 8.

34. The method of any of claims 1-23, wherein the RRM3-derived peptide of CAPERα comprises the amino acid sequence of SEQ ID NO: 9.

35. The method any of claims 1-23, wherein the RRM3-derived peptide of CAPERα comprises the amino acid sequence of SEQ ID NO: 10.

36. The method of any of claims 1-23, wherein the RRM3-derived peptide of CAPERα comprises the amino acid sequence of SEQ ID NO: 11.

37. The method of any of claims 1-36, wherein the RRM3-derived peptide of CAPERα is conjugated with the Penetratin cell penetrating peptide.

38. The method of claim 37, wherein the Penetratin cell penetrating peptide comprises the amino acid sequence of SEQ ID NO: 12.

39. The method of any of claims 1-38, wherein the RRM3-derived peptide of CAPERα is conjugated to a poly-histidine peptide.

40. The method of any of claims 1-39, wherein the RRM3-derived peptide of CAPERα is pegylated.

41. The method of any of claims 1-40, wherein the RRM3-derived peptide of CAPERα is myristoylated.

42. An RRM3-derived peptide of CAPERα conjugated to a protein transduction domain.

43. The RRM3-derived peptide of CAPERα of claim 42, wherein the RRM3-derived peptide of CAPERα does not include the 5′ n-sheet of the RRM3 domain of CAPERα.

44. The RRM3-derived peptide of CAPERα of any of claims 42-43, wherein the RRM3-derived peptide of CAPERα does not include the 5′ α-helix of the RRM3 domain of CAPERα.

45. The RRM3-derived peptide of CAPERα of claim 42, wherein the RRM3-derived peptide of CAPERα comprises the amino acid sequence of SEQ ID NO: 1.

46. The RRM3-derived peptide of CAPERα of claim 42, wherein the RRM3-derived peptide of CAPERα comprises the amino acid sequence of SEQ ID NO: 2.

47. The RRM3-derived peptide of CAPERα of claim 42, wherein the RRM3-derived peptide of CAPERα comprises the amino acid sequence of SEQ ID NO: 3.

48. The RRM3-derived peptide of CAPERα of claim 42, wherein the RRM3-derived peptide of CAPERα comprises the amino acid sequence of SEQ ID NO: 4.

49. The RRM3-derived peptide of CAPERα of claim 42, wherein the RRM3-derived peptide of CAPERα comprises the amino acid sequence of SEQ ID NO: 5.

50. The RRM3-derived peptide of CAPERα of claim 42, wherein the RRM3-derived peptide of CAPERα comprises the amino acid sequence of SEQ ID NO: 6.

51. The RRM3-derived peptide of CAPERα of claim 42, wherein the RRM3-derived peptide of CAPERα comprises the amino acid sequence of SEQ ID NO: 7.

52. The RRM3-derived peptide of CAPERα of claim 42, wherein the RRM3-derived peptide of CAPERα comprises the amino acid sequence of SEQ ID NO: 8.

53. The RRM3-derived peptide of CAPERα of claim 42, wherein the RRM3-derived peptide of CAPERα comprises the amino acid sequence of SEQ ID NO: 9.

54. The RRM3-derived peptide of CAPERα of claim 42, wherein the RRM3-derived peptide of CAPERα comprises the amino acid sequence of SEQ ID NO: 10.

55. The RRM3-derived peptide of CAPERα of claim 42, wherein the RRM3-derived peptide of CAPERα comprises the amino acid sequence of SEQ ID NO: 11.

56. The RRM3-derived peptide of CAPERα of any of claims 42-55, wherein the RRM3-derived peptide of CAPERα is conjugated with the Penetratin cell penetrating peptide.

57. The RRM3-derived peptide of CAPERα of claim 56, wherein the Penetratin cell penetrating peptide comprises the amino acid sequence of SEQ ID NO: 12.

58. The RRM3-derived peptide of CAPERα of any of claims 42-57, wherein the RRM3-derived peptide of CAPERα is conjugated to a poly-histidine peptide.

59. The RRM3-derived peptide of CAPERα of any of claims 42-58, wherein the RRM3-derived peptide of CAPERα is pegylated.

60. The RRM3-derived peptide of CAPERα of any of claims 42-59, wherein the RRM3-derived peptide of CAPERα is myristoylated.


SEQUENCE LISTING (117 sequences)

SEQ ID NO: 1 - protein, 20 aa, Homo sapiens
GWDTEIKDDVIEECNKHGGV

SEQ ID NO: 2 - protein, 32 aa, Homo sapiens
EIKDDVIEECNKHGGVIHIYVDKNSAQGNVYV

SEQ ID NO: 3 - protein, 18 aa, Homo sapiens
LHGRWFAGKMITAAYVPL

SEQ ID NO: 4 - protein, 64 aa, Artificial Sequence
  RRM3-derived synthetic sequence
GWDTIEECNKHGGVIHIYVDKNSAQGNVYVKCPSIAAAIAAVNALHGRWFAGKMITAAYVPLPT

SEQ ID NO: 5 - protein, 64 aa, Artificial Sequence
  RRM3-derived synthetic sequence
GWDTEIKDDVIEGVIHIYVDKNSAQGNVYVKCPSIAAAIAAVNALHGRWFAGKMITAAYVPLPT

SEQ ID NO: 6 - protein, 66 aa, Artificial Sequence
  RRM3-derived synthetic sequence
GWDTEIKDDVIEECNKHGGVIHKNSAQGNVYVKCPSIAAAIAAVNALHGRWFAGKMITAAYVPLPT

SEQ ID NO: 7 - protein, 65 aa, Artificial Sequence
  RRM3-derived synthetic sequence
GWDTEIKDDVIEECNKHGGVIHIYVDKNSAQKCPSIAAAIAAVNALHGRWFAGKMITAAYVPLPT

SEQ ID NO: 8 - protein, 64 aa, Artificial Sequence
  RRM3-derived synthetic sequence
GWDTEIKDDVIEECNKHGGVIHIYVDKNSAQGNVYVKCPSIAAAIAAVNAAGKMITAAYVPLPT

SEQ ID NO: 9 - protein, 66 aa, Artificial Sequence
  RRM3-derived synthetic sequence
GWDTEIKDDVIEECNKHGGVIHIYVDKNSAQGNVYVKCPSIAAAIAAVNALHGRWFAGKMITPLPT

SEQ ID NO: 10 - protein, 20 aa, Homo sapiens
IHYVDKNSAQGNVYVKKCPS

SEQ ID NO: 11 - protein, 32 aa, Artificial Sequence; identical to SEQ ID NO: 2
  RRM3-derived synthetic sequence

SEQ ID NO: 12 - protein, 18 aa, Drosophila melanogaster
RQIKIWFQNRRMKWKKGG

SEQ ID NO: 13 - protein, 70 aa, Homo sapiens
GWDTEIKDDVIEECNKHGGVIHIYVDKNSAQGNVYVKCPSIAAAIAAVNALHGRWFAGKMITAAYVPLPT

SEQ ID NO: 14 - protein, 56 aa, Artificial Sequence
  RRM3-derived synthetic sequence
RQIKIWFQNRRMKWKKGGEIKDDVIEECNKHGGVIHIYVDKNSAQGNVYVHHHHHH

SEQ ID NO: 15 - protein, 50 aa, Artificial Sequence
  RRM3-derived synthetic sequence
RQIKIWFQNRRMKWKKGGEIKDDVIEECNKHGGVIHIYVDKNSAQGNVYV

SEQ ID NO: 16 - protein, 50 aa, Artificial Sequence; identical to SEQ ID NO: 15
  RRM3-derived synthetic sequence

SEQ ID NO: 17 - protein, 56 aa, Artificial Sequence; identical to SEQ ID NO: 14
  RRM3-derived synthetic sequence

SEQ ID NO: 18 - protein, 18 aa, Artificial Sequence
  RRM3-derived synthetic sequence
  MISC_FEATURE (18)..(18): note  residue 18 is connected at its 3′ end with a spacer, 11-amino-3,6,9-trioxaundecanoic acid, which is in turn connected to the 5′ end of the amino acid reported in this sequence listing as SEQ ID NO 117.
RQIKIWFQNRRMKWKKGG

SEQ ID NO: 19 - protein, 56 aa, Artificial Sequence; identical to SEQ ID NO: 14
  RRM3-derived synthetic sequence
  MISC_FEATURE (1)..(1): note  residue 1 is attached at its 5′ end to n-tetradecanoic acid

SEQ ID NO: 20 - protein, 38 aa, Artificial Sequence
  RRM3-derived synthetic sequence
RQIKIWFQNRRMKWKKGGGWDTEIKDDVIEECNKHGGV

SEQ ID NO: 21 - protein, 44 aa, Artificial Sequence
  RRM3-derived synthetic sequence
RQIKIWFQNRRMKWKKGGGWDTEIKDDVIEECNKHGGVHHHHHH

SEQ ID NO: 22 - protein, 37 aa, Artificial Sequence
  RRM3-derived synthetic sequence
RQIKIWFQNRRMKWKKGIHYVDKNSAQGNVYVKKCPS

SEQ ID NO: 23 - protein, 44 aa, Artificial Sequence
  RRM3-derived synthetic sequence
RQIKIWFQNRRMKWKKGGIHYVDKNSAQGNVYVKKCPSHHHHHH

SEQ ID NO: 24 - protein, 36 aa, Artificial Sequence
  RRM3-derived synthetic sequence
RQIKIWFQNRRMKWKKGGLHGRWFAGKMITAAYVPL

SEQ ID NO: 25 - protein, 42 aa, Artificial Sequence
  RRM3-derived synthetic sequence
RQIKIWFQNRRMKWKKGGLHGRWFAGKMITAAYVPLHHHHHH

SEQ ID NO: 26 - protein, 44 aa, Artificial Sequence
  RRM3-derived synthetic sequence
GRKKRRQRRRPPEIKDDVIEECNKHGGVIHIYVDKNSAQGNVYV

SEQ ID NO: 27 - protein, 50 aa, Artificial Sequence
  RRM3-derived synthetic sequence
GRKKRRQRRRPPEIKDDVIEECNKHGGVIHIYVDKNSAQGNVYVHHHHHH

SEQ ID NO: 28 - protein, 48 aa, Artificial Sequence
  RRM3-derived synthetic sequence
KLALKLALKALKAALKEIKDDVIEECNKHGGVIHIYVDKNSAQGNVYV

SEQ ID NO: 29 - protein, 54 aa, Artificial Sequence
  RRM3-derived synthetic sequence
KLALKLALKALKAALKEIKDDVIEECNKHGGVIHIYVDKNSAQGNVYVHHHHHH

SEQ ID NO: 30 - protein, 60 aa, Homo sapiens
GWDTEIKDDVIEECNKHGGVIHIYVDKNSAQGNVYVKCPSIAAAIAAVNALHGRWFAGKM

SEQ ID NO: 31 - protein, 78 aa, Artificial Sequence
  RRM3-derived synthetic sequence
RQIKIWFQNRRMKWKKGGGWDTEIKDDVIEECNKHGGVIHIYVDKNSAQGNVYVKCPSIAAAIAAVNALHGRWFAGKM

SEQ ID NO: 32 - protein, 92 aa, Artificial Sequence
  RRM3-derived synthetic sequence
RQIKIWFQNRRMKWKKGWDTEIKDDVIEECNKHGGVIHIYVDKNSAQGNVYVKCPSIAAAIAAVNALHGRWFAGKMTAAYVPEQKLISEEDL

SEQ ID NO: 33 - protein, 76 aa, Artificial Sequence
  RRM3-derived synthetic sequence
GWDTEIKDDVIEECNKHGGVIHIYVDKNSAQGNVYVKCPSIAAAIAAVNALHGRWFAGKMTAAYVPEQKLISEEDL

SEQ ID NO: 34 - protein, 64 aa, Homo sapiens
EIKDDVIEECNKHGGVIHIYVDKNSAQGNVYVKCPSIAAAIAAVNALHGRWFAGKMITAAYVPL

SEQ ID NO: 35 - protein, 82 aa, Artificial Sequence
  RRM3-derived synthetic sequence
RQIKIWFQNRRMKWKKGGGWDTIEECNKHGGVIHIYVDKNSAQGNVYVKCPSIAAAIAAVNALHGRWFAGKMITAAYVPLPT

SEQ ID NO: 36 - protein, 88 aa, Artificial Sequence
  RRM3-derived synthetic sequence
RQIKIWFQNRRMKWKKGGGWDTIEECNKHGGVIHIYVDKNSAQGNVYVKCPSIAAAIAAVNALHGRWFAGKMITAAYVPLPTHHHHHH

SEQ ID NO: 37 - protein, 82 aa, Artificial Sequence
  RRM3-derived synthetic sequence
RQIKIWFQNRRMKWKKGGGWDTEIKDDVIEGVIHIYVDKNSAQGNVYVKCPSIAAAIAAVNALHGRWFAGKMITAAYVPLPT

SEQ ID NO: 38 - protein, 85 aa, Artificial Sequence
  RRM3-derived synthetic sequence
RQIKIWFQNRRMKWKKGGGWDTEIKDDVIEGVIHIYVDKNSAQGNVYVKCPSIAAAIAAVNALHGRWFAGKMITAAYVPHHHHHH

SEQ ID NO: 39 - protein, 84 aa, Artificial Sequence
  RRM3-derived synthetic sequence
RQIKIWFQNRRMKWKKGGGWDTEIKDDVIEECNKHGGVIHKNSAQGNVYVKCPSIAAAIAAVNALHGRWFAGKMITAAYVPLPT

SEQ ID NO: 40 - protein, 90 aa, Artificial Sequence
  RRM3-derived synthetic sequence
RQIKIWFQNRRMKWKKGGGWDTEIKDDVIEECNKHGGVIHKNSAQGNVYVKCPSIAAAIAAVNALHGRWFAGKMITAAYVPLPTHHHHHH

SEQ ID NO: 41 - protein, 83 aa, Artificial Sequence
  RRM3-derived synthetic sequence
RQIKIWFQNRRMKWKKGGGWDTEIKDDVIEECNKHGGVIHIYVDKNSAQKCPSIAAAIAAVNALHGRWFAGKMITAAYVPLPT

SEQ ID NO: 42 - protein, 89 aa, Artificial Sequence
  RRM3-derived synthetic sequence
RQIKIWFQNRRMKWKKGGGWDTEIKDDVIEECNKHGGVIHIYVDKNSAQKCPSIAAAIAAVNALHGRWFAGKMITAAYVPLPTHHHHHH

SEQ ID NO: 43 - protein, 82 aa, Artificial Sequence
  RRM3-derived synthetic sequence
RQIKIWFQNRRMKWKKGGGWDTEIKDDVIEECNKHGGVIHIYVDKNSAQGNVYVKCPSIAAAIAAVNAAGKMITAAYVPLPT

SEQ ID NO: 44 - protein, 88 aa, Artificial Sequence
  RRM3-derived synthetic sequence
RQIKIWFQNRRMKWKKGGGWDTEIKDDVIEECNKHGGVIHIYVDKNSAQGNVYVKCPSIAAAIAAVNAAGKMITAAYVPLPTHHHHHH

SEQ ID NO: 45 - protein, 84 aa, Artificial Sequence
  RRM3-derived synthetic sequence
RQIKIWFQNRRMKWKKGGGWDTEIKDDVIEECNKHGGVIHIYVDKNSAQGNVYVKCPSIAAAIAAVNALHGRWFAGKMITPLPT

SEQ ID NO: 46 - protein, 90 aa, Artificial Sequence
  RRM3-derived synthetic sequence
RQIKIWFQNRRMKWKKGGGWDTEIKDDVIEECNKHGGVIHIYVDKNSAQGNVYVKCPSIAAAIAAVNALHGRWFAGKMITPLPTHHHHHH

SEQ ID NO: 47 - protein, 24 aa, Artificial Sequence
  RRM3-derived synthetic sequence
RQIKIWFQNRRMKWKKGGHHHHHH

SEQ ID NO: 48 - DNA, 63 bp, Artificial Sequence
  RRM3-derived synthetic sequence
gatccaagaagtcagagtgcgaagtctcaagaggacttcgcactctgacttcttttttttgga

SEQ ID NO: 49 - DNA, 63 bp, Artificial Sequence
  RRM3-derived synthetic sequence
agcttccaaaaaaaagaagtcagagtgcgaagtcctcttgagacttcgcactctgacttcttg

SEQ ID NO: 50 - RNA, 23 nt, Artificial Sequence
  RRM3-derived synthetic sequence
aauucuccgaacgugucacguuu

SEQ ID NO: 51 - RNA, 23 nt, Artificial Sequence
  RRM3-derived synthetic sequence
acgugacacguucggagaauuuu

SEQ ID NO: 52 - RNA, 21 nt, Artificial Sequence
  RRM3-derived synthetic sequence
gacagaaauucaagacguuuu

SEQ ID NO: 53 - RNA, 21 nt, Artificial Sequence
  RRM3-derived synthetic sequence
aacgucuugaauuucugucuu

SEQ ID NO: 54 - RNA, 21 nt, Artificial Sequence
  RRM3-derived synthetic sequence
gaagcgaaguagagacagauu

SEQ ID NO: 55 - RNA, 21 nt, Artificial Sequence
  RRM3-derived synthetic sequence
ucugucucuacuucgcuucuu

SEQ ID NO: 56 - RNA, 23 nt, Artificial Sequence
  RRM3-derived synthetic sequence
aagauuggguugccucauauuuu

SEQ ID NO: 57 - RNA, 23 nt, Artificial Sequence
  RRM3-derived synthetic sequence
aauaugaggcaacccaaucuuuu

SEQ ID NO: 58 - RNA, 21 nt, Artificial Sequence
  RRM3-derived synthetic sequence
aagaagucagagugcgaaguc

SEQ ID NO: 59 - DNA, 19 bp, Artificial Sequence
  RRM3-derived synthetic sequence
ggacaagagtagagagaga

SEQ ID NO: 60 - DNA, 20 bp, Artificial Sequence
  RRM3-derived synthetic sequence
cggaacaggcgtttagagaa

SEQ ID NO: 61 - DNA, 20 bp, Artificial Sequence
  RRM3-derived synthetic sequence
tggcactgctcaacttgttc

SEQ ID NO: 62 - DNA, 22 bp, Artificial Sequence
  RRM3-derived synthetic sequence
cagctatcctctcagatccatc

SEQ ID NO: 63 - DNA, 22 bp, Artificial Sequence
  RRM3-derived synthetic sequence
ccttgtctttccggactttctg

SEQ ID NO: 64 - DNA, 24 bp, Artificial Sequence
  RRM3-derived synthetic sequence
ggctttctctgatgtaattcttgc

SEQ ID NO: 65 - DNA, 22 bp, Artificial Sequence
  RRM3-derived synthetic sequence
gtattttggtctgactgcttgc

SEQ ID NO: 66 - DNA, 27 bp, Artificial Sequence
  RRM3-derived synthetic sequence
tggatctgaagaaatacttggattcta

SEQ ID NO: 67 - DNA, 20 bp, Artificial Sequence
  RRM3-derived synthetic sequence
caatcccctgtaggatttgg

SEQ ID NO: 68 - DNA, 21 bp, Artificial Sequence
  RRM3-derived synthetic sequence
ctgcatttggctgtgaactac

SEQ ID NO: 69 - DNA, 21 bp, Artificial Sequence
  RRM3-derived synthetic sequence
acaaactctgctacttctggg

SEQ ID NO: 70 - DNA, 20 bp, Artificial Sequence
  RRM3-derived synthetic sequence
ctgcagatggagcatgttgt

SEQ ID NO: 71 - DNA, 20 bp, Artificial Sequence
  RRM3-derived synthetic sequence
tcttcacgaggaggcttgat

SEQ ID NO: 72 - DNA, 30 bp, Artificial Sequence
  RRM3-derived synthetic sequence
gcatgaggaaacgcatcgctgcctccaagt

SEQ ID NO: 73 - DNA, 30 bp, Artificial Sequence
  RRM3-derived synthetic sequence
gcgaccaagtccttcccactcgtgcacact

SEQ ID NO: 74 - DNA, 21 bp, Artificial Sequence
  RRM3-derived synthetic sequence
aaggcggaggagacctgcgcg

SEQ ID NO: 75 - DNA, 18 bp, Artificial Sequence
  RRM3-derived synthetic sequence
atcgtgcggcattgcggc

SEQ ID NO: 76 - DNA, 19 bp, Artificial Sequence
  RRM3-derived synthetic sequence
tgtctgcagccaggagagc

SEQ ID NO: 77 - DNA, 22 bp, Artificial Sequence
  RRM3-derived synthetic sequence
atgcatcaaacaactgtggcca

SEQ ID NO: 78 - DNA, 22 bp, Artificial Sequence
  RRM3-derived synthetic sequence
gtccctctaaagcagctcaaaa

SEQ ID NO: 79 - DNA, 22 bp, Artificial Sequence
  RRM3-derived synthetic sequence
ctcccttcaaaagatccaactg

SEQ ID NO: 80 - DNA, 23 bp, Artificial Sequence
  RRM3-derived synthetic sequence
cctgaggccatttctttcataga

SEQ ID NO: 81 - DNA, 23 bp, Artificial Sequence
  RRM3-derived synthetic sequence
gtcacagtgactgagcggctaat

SEQ ID NO: 82 - DNA, 23 bp, Artificial Sequence
  RRM3-derived synthetic sequence
gatctatgaataagcctttggag

SEQ ID NO: 83 - DNA, 21 bp, Artificial Sequence
  RRM3-derived synthetic sequence
tttcgtcctggtatattcggt

SEQ ID NO: 84 - DNA, 20 bp, Artificial Sequence
  RRM3-derived synthetic sequence
acaaacccatgtttcgaagg

SEQ ID NO: 85 - DNA, 20 bp, Artificial Sequence
  RRM3-derived synthetic sequence
agtgtccaatggtggaggac

SEQ ID NO: 86 - DNA, 22 bp, Artificial Sequence
  RRM3-derived synthetic sequence
aggatgagctggctagagagac

SEQ ID NO: 87 - DNA, 21 bp, Artificial Sequence
  RRM3-derived synthetic sequence
cccaccccacccctgtactaa

SEQ ID NO: 88 - DNA, 26 bp, Artificial Sequence
  RRM3-derived synthetic sequence
gggagacgccaagacagacaagatcg

SEQ ID NO: 89 - DNA, 24 bp, Artificial Sequence
  RRM3-derived synthetic sequence
ggtgaagaccaagaaggcctcagg

SEQ ID NO: 90 - DNA, 20 bp, Artificial Sequence
  RRM3-derived synthetic sequence
cccttggtgttttggagaaa

SEQ ID NO: 91 - DNA, 24 bp, Artificial Sequence
  RRM3-derived synthetic sequence
gagtgtttcttatggtgaagtcca

SEQ ID NO: 92 - DNA, 21 bp, Artificial Sequence
  RRM3-derived synthetic sequence
ggccaaattcagataatcgac

SEQ ID NO: 93 - DNA, 20 bp, Artificial Sequence
  RRM3-derived synthetic sequence
ccacttcgtagcatttacgg

SEQ ID NO: 94 - DNA, 19 bp, Artificial Sequence
  RRM3-derived synthetic sequence
gctggtgaaaaggacctct

SEQ ID NO: 95 - DNA, 20 bp, Artificial Sequence
  RRM3-derived synthetic sequence
cacaggactagaacacctgc

SEQ ID NO: 96 - DNA, 21 bp, Artificial Sequence
  RRM3-derived synthetic sequence
ccatcctcaagaaggtgttgg

SEQ ID NO: 97 - DNA, 23 bp, Artificial Sequence
  RRM3-derived synthetic sequence
gtgtcccatatccgcaattttat

SEQ ID NO: 98 - DNA, 19 bp, Artificial Sequence
  RRM3-derived synthetic sequence
aaactgggtcgcattgtgg

SEQ ID NO: 99 - DNA, 20 bp, Artificial Sequence
  RRM3-derived synthetic sequence
tctcggctgctgcattgttc

SEQ ID NO: 100 - DNA, 20 bp, Artificial Sequence
  RRM3-derived synthetic sequence
tgcagctatacccaggctgg

SEQ ID NO: 101 - DNA, 20 bp, Artificial Sequence
  RRM3-derived synthetic sequence
tgcagctatacccaggctgg

SEQ ID NO: 102 - DNA, 20 bp, Artificial Sequence
  RRM3-derived synthetic sequence
cctgctttgtcctttgatgc

SEQ ID NO: 103 - DNA, 21 bp, Artificial Sequence
  RRM3-derived synthetic sequence
tgagtcaatccctggaaagtc

SEQ ID NO: 104 - DNA, 20 bp, Artificial Sequence
  RRM3-derived synthetic sequence
cggaagagtgtctggagcaa

SEQ ID NO: 105 - DNA, 19 bp, Artificial Sequence
  RRM3-derived synthetic sequence
ggatgaagcggagtctgga

SEQ ID NO: 106 - DNA, 23 bp, Artificial Sequence
  RRM3-derived synthetic sequence
tgctgacaatcatcaccaagttc

SEQ ID NO: 107 - DNA, 20 bp, Artificial Sequence
  RRM3-derived synthetic sequence
tctcccttcgtgtcctggaa

SEQ ID NO: 108 - DNA, 20 bp, Artificial Sequence
  RRM3-derived synthetic sequence
tgggttcgagttctttgagg

SEQ ID NO: 109 - DNA, 20 bp, Artificial Sequence
  RRM3-derived synthetic sequence
gtccaacgactcggacatct

SEQ ID NO: 110 - DNA, 23 bp, Artificial Sequence
  RRM3-derived synthetic sequence
ttctgttgatcccacttcactgt

SEQ ID NO: 111 - DNA, 22 bp, Artificial Sequence
  RRM3-derived synthetic sequence
acacacatgcacacacactaac

SEQ ID NO: 112 - DNA, 20 bp, Artificial Sequence
  RRM3-derived synthetic sequence
aaggacctcggctggaagtg

SEQ ID NO: 113 - DNA, 20 bp, Artificial Sequence
  RRM3-derived synthetic sequence
cccgggttatgctggttgta

SEQ ID NO: 114 - DNA, 29 bp, Artificial Sequence
  RRM3-derived synthetic sequence
gctcgctcaacgtcaacctcaactcggtg

SEQ ID NO: 115 - DNA, 29 bp, Artificial Sequence
  RRM3-derived synthetic sequence
agtggcggctgccctggtccagcaccacc

SEQ ID NO: 116 - protein, 92 aa, Artificial Sequence
  RRM3-derived synthetic sequence
RQIKIWFQNRRMKWKKRTVFCMQLAARIRPRDLEEFFSTVGKVRDVRMISDRNSRRSKGIAYVEFVDVSSVPLAIMTAAYVPEQKLISEEDL

SEQ ID NO: 117 - protein, 38 aa, Artificial Sequence
  RRM3-derived synthetic sequence
  MISC_FEATURE (1)..(1): note amino acid 1 is connected at its 5′ end to a spacer, 11-amino-3,6,9-trioxaundecanoic acid, which is connected to the 3′ end of the amino acid sequence included in this sequence listing as SEQ ID NO 18
EIKDDVIEECNKHGGVIHIYVDKNSAQGNVYVHHHHHH